(19) United States
(12) Patent Application Publication
(10) Pub. No.: US 2023/0304047 A1
(43) Pub. Date: Sep. 28, 2023

(54) IMPROVED GENE EDITING

(71) Applicants: University of Oslo, Oslo (NO); University of Helsinki, Helsink (FI)

(72) Inventors: Emma Haapaniemi, Oslo (NO); Jussi Taipale, Cambridge (GB); Ganna Reint, Oslo (NO)

(21) Appl. No.: 18/020,720

(22) PCT Filed: Aug. 11, 2021
(86) PCT No.: PCT/IB2021/000550

§ 371 Date: Feb. 10, 2023

Related U.S. Application Data
(60) Provisional application No. 63/064,047, filed on Aug. 11, 2020.

(51) Int. Cl. C12N 15/90; C12N 15/11; C12N 9/22

(52) U.S. Cl. CPC C12N 15/907; C12N 15/11; C12N 9/22; C12N 2310/20; C12N 2800/80

(57) ABSTRACT

The present invention generally relates to compositions and methods for improved gene editing. In particular, the present invention relates to compositions and methods for using nucleic acid repair proteins to improve the outcomes of gene editing.

### FIELD OF THE INVENTION

The present invention generally relates to compositions and methods for improved gene editing. In particular, the present invention relates to compositions and methods for using nucleic acid repair proteins to improve the outcomes of gene editing.

### BACKGROUND OF THE INVENTION

Genome editing is a powerful technology that allows for the specific and often precise addition or removal of genetic material. Genome editing is initiated by making double stranded DNA breaks in the target cell. These double stranded DNA breaks can be created by several methods—including; meganucleases, Zinc-Finger Nucleases, TALE-nucleases, and/or the CRISPR/Cas9 restriction modification system. Each of these systems creates a dsDNA break at a user designated genomic location. After the creation of the dsDNA break, the cellular machinery acts quickly to repair this dsDNA using either by the non-homologous end joining (NHEJ) pathway or by homologous recombination (HDR). While, the NHEJ pathway efficiently repairs this break, repair is frequently imperfect resulting in insertions and deletions. If these insertions and deletions created by NHEJ repair occur within open reading frames, the most common result is a frame-shift mutation. This frame shift often results in the inactivation of that particular gene. Repair of the dsDNA break by HDR pathway not only can result in precise repair but also allows for the introduction of experimentally designed genomic elements. The correction of many diseases, successful gene therapy, can be achieved by forcing the cell to correct the dsDNA break using HDR. Unfortunately for gene therapy researchers, clinicians, and patients, most human cells strongly prefer to correct dsDNA breaks the error-prone NHEJ pathway as opposed to the more precise HDR pathway. Using endogenous cellular machinery 95% of dsDNA breaks are repaired using NHEJ, while only 5% of dsDNA breaks are repaired using HDR. This statistic represents the best-case scenario; many cell types lack HDR machinery altogether resulting in no repair using the precise HDR pathway. For precise gene therapy to be successful, a cells ability to use the HDR pathway must be improved.

### SUMMARY OF THE INVENTION

The present invention generally relates to compositions and methods for improved gene editing. In particular, the present invention relates to compositions and methods for using nucleic acid repair proteins to improve the outcomes of gene editing.

Experiments described herein studied the effect of 450 DNA repair protein-Cas9 fusions on CRISPR genome editing outcomes. Exemplary DNA repair (e.g., replicating) proteins that enhanced editing were identified. Such DNA replicating proteins find use in enhancing gene editing in vitro, ex vivo, and in vivo for a variety of research, screening, and therapeutic applications.

For example, in some embodiments, provided herein is a composition, comprising: a nucleic acid encoding a fusion protein comprising a Cas9 polypeptide fused to a nucleic acid repair protein.

In further embodiments, provided herein is a composition, comprising a) a first nucleic acid encoding a Cas9 polypeptide and a second nucleic acid encoding a nucleic acid repair protein.

The present disclosure is not limited to particular nucleic acid repair proteins. In some embodiments, the nucleic acid repair protein is a replicative polymerase (e.g., a DNA polymerase or an RNA polymerase). In some embodiments, the polymerase is a human polymerase. In some embodiments, the nucleic acid repair protein is a DNA replication factor. Examples include but are not limited to a DNA polymerase delta (e.g., DNA polymerase delta III (POLD3)), POLN, rfc4, rfc5, POLR2H, SIRT6, or PAPD7.

In some embodiments, the nucleic acid is a vector. In some embodiments, the first and second nucleic acid are on the same or different vectors. For example, in some embodiments, the first and second nucleic acid are on the same vector and are separated by an internal ribosome entry site (IRES). Examples of vectors include but are not limited to, viral vectors and plasmids

Additional embodiments provide a fusion protein or pair of polypeptides encoded by the nucleic acids described herein.

Yet other embodiments provide a kit or system, comprising: a) a nucleic acid, fusion protein, or pair of polypeptides described herein; and b) a plurality (e.g., one or two) of guide RNAs (e.g., sgRNAs). In some embodiments, the kit or system further comprises a nucleic acid encoding an exogenous gene of interest (e.g., on a vector).

Certain embodiments provide a method (e.g., gene editing method), comprising: introducing a system described herein into a cell. In some embodiments, the introducing results in disruption, deletion, or insertion of a target nucleic acid (e.g., gene) in the cell. In some embodiments, the gene editing results in an increase or decrease in expression of an endogenous or exogenous gene in the cell. In some embodiments, the cell is a eukaryotic cell (e.g., a mammalian (e.g., human) cell). In some embodiments, the cell is in vitro, ex vivo, or in vivo. In some embodiments, the method treats a disease or condition in a subject. In some embodiments, the gene editing comprising HDR or NHEJ.

Other embodiments provide a cell comprising a system described herein.

Also provided herein is the use of a system described herein to alter expression of gene in a target cell or edit the genome of a target cell.

Additional embodiments are described herein.

### BRIEF DESCRIPTION OF THE DRAWINGS

The figures herein are for illustrative purposes only and are not necessarily drawn to scale.

FIG. 1. DNA repair proteins that affect genome editing outcomes. a. Schematic representation of the screen. b. Normalized GFP recovery values from two independent screens (n=3). c. Experiment average—normalized GFP recovery values for 60 fusions, chosen based on their performance in (b). In HEK293T n=4, one independent biological experiment, in RPE1 n=2, two independent biological experiments. Statistical significance is calculated as in (b). d. Normalized GFP recovery values for ten best-performing and five worst-performing Cas9 fusions from (c). e. Experiment average—normalized HDR editing for 35 fusions in four endogenous loci (ELANE, RNF2, Enh 4-1 and CTCF1), quantified by droplet digital PCR. Polymerase fusions are marked with red pointers. n=3, one independent biological experiment for each locus. Statistical significance is calculated as in (b) f. Raw HDR (%) values and HDR ratios to the total editing (%), for the best-performing Cas9 fusions from (e). Error bars represent standard deviations. g. Illustration of the DNA replication fork. POLD3, RCF4 and RFC5 localization is marked with red arrows.

FIG. 2. Protein-protein interactions of editing-enhancing Cas9 fusions a. Schematic representation of the AP-MS experimental workflow. b. Protein interaction map of the Cas9 fusions (three biological replicates).

FIG. 3. Cas9-POLD3 editing efficiency across different conditions. a. Fusion protein performance across different plasmid concentrations in reporter RPE-1 cells. n=4, representative of two independent experiments, bar denotes mean value, error bars represent ±S.D. b. GFP reporter locus editing efficiency of recombinant Cas9 fusion proteins in RPE-1 and HEK293T cells. n=5, one of two independent experiments, bar denotes mean value, error bars represent ±S.D. c. Cas9-POLD3 mRNA editing efficiency across 5 endogenous target loci in immortalized fibroblasts. n=3, data points from two independent experiments. Bar denotes mean value, error bars represent ±S.D. d. Schematic representation of the cell-cycle dependent CRISPR inhibitor (AcrIIA2-Cdt1 fusion protein). e. AcrllA2-Cdt1 performance with Cas9WT in reporter RPE1 cells with functional and non-functional p53 gene. f. AcrllA2-Cdt1 performance with Cas9WT and Cas9-POLD3 plasmids in reporter RPE1 cells. n=4, one independent experiment, bar denotes mean value, error bars represent ±S.D.

FIG. 4. Cas9 and DNA repair protein co-expression screen. a. Screen schematic. Reporter HEK293T cells were co-transfected with repair DNA and two types of plasmids: one encoding Cas9WT and another encoding a protein fused to the MS2 tag. b. Mean GFP values for the tested repair proteins plotted against statistical significance (n=3, one biological experiment, one-way Anova testing against the experiment average). c. Plate-normalized GFP mean values plotted against statistical significance (n=3, one biological experiment, one-way Anova testing against the experiment average). d. Plate-normalized GFP values from the top and bottom DNA repair proteins that either worsen or improve GFP repair.

FIG. 5. HDR efficiency of the best-performing fusions. Each cell in the heatmap illustrates an HDR value that is normalized to the average HDR efficiency of the whole experiment (“fold change”). a. Normalized GFP conversion rate in HEK293T (n=4 one independent biological experiment) and RPE (n=2, two independent biological experiments) reporter cell lines. b. Normalized HDR editing in HEK293T cells that stably express guides targeting endogenous loci (ELANE, RNF2, Enh4-1 and CTCF1) (n=3, one independent biological experiment for each locus).

FIG. 6. HDR efficiency of the best-performing fusions. a-d.: % of HDR and % HDR of total editing in HEK293T cells that stably express guides targeting endogenous loci (ELANE, RNF2, Enh 4-1 and CTCF1).

FIG. 7. Cas9 plasmid and mRNA editing efficiencies in normal cells. a. % NHEJ in immortalized fibroblast cell line BJ-5ta. b. Representative light microscopy captures of BJ-5ta-RNF2-sgRNA cells transfected with Cas9-CCNH plasmid and mRNA.

FIG. 8. Cas9 fusion editing efficiency in immortalized fibroblasts that stably express RNF2-targeting sgRNA (BJ-5ta-RNF2-sgRNA cells). a. HDR and NHEJ editing efficiency four nights after Cas9 mRNA electroporation. b. Cas9 fusion mRNAs on a denaturing RNA gel.

FIG. 9. Cell cycle timer (AcrIIA2-Cdt1 fusion protein) in immortalized fibroblasts and HEK293T cells.

FIG. 10. FACS gating strategy a-b. HEK293T reporter cell line carrying mGFP-RFP color cassette. c-d. RPE-1 reporter cell line carrying mGFP-RFP color cassette. e-f. RPE-1 reporter cell line carrying mGFP-BFP color cassette.

FIG. 11. Quantifying CRISPR editing by droplet digital PCR (ddPCR).

FIG. 12. Examples of the ddPCR gating for quantifying HDR and NHEJ. For each set, the droplet distribution in the negative control is shown on the left and the CRISPR-edited samples on the right.

FIG. 13. Exemplary sequences utilized in the Examples.

FIG. 14. Graphical depiction of data related to validation of Cas9 fusions for editing in human fibroblasts.

FIG. 15. Graphs providing validation of the use of Cas9 fusions for editing in retinal pigment epithelium (RPE) cells.

FIG. 16. Graphs providing a comparison of editing efficiency of the Cas9-POLD3 fusion versus published Cas9 fusions.

FIG. 17. Graphs showing that Cas9-POLD3 fusions work at lower concentrations that wild-type Cas9.

FIG. 18. Graphs showing editing efficiency of Cas9-POLD3 fusions in human embryonic stem cells and peripheral blood mononuclear cells.

FIG. 19. Graph providing data on PD1 knockout in peripheral blood mononuclear cells with a Cas9-POLD3 fusion vs. wildtype Cas9.

FIG. 20. Graph providing data on BCL11A knockout in CD34+ cells with a Cas9-POLD3 fusion vs wildtype Cas9.

FIG. 21. Graphical depiction of data related to quantification of DNA damage markers.

### DETAILED DESCRIPTION OF THE INVENTION

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. These and other embodiments are disclosed or are obvious from and encompassed by, the following Detailed Description.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In aspects of the invention the terms “chimeric RNA”, “chimeric guide RNA”, “guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”. The term “tracr mate sequence” may also be used interchangeably with the term “direct repeat(s)”. Exemplary CRISPR-Cas system are provided in U.S. Pat. No. 8,697,359 and US 20140234972, both of which are incorporated herein by reference in their entirety.

As used herein, the term “filament” refers to a single stranded nucleic acid having a multimeric recombinase complex bound thereto. In some embodiments, the filament may be “isolated” and provided in a biologically compatible solution such as a buffered solution.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self 17 hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif (1990). Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerivisae include pYepSec1 (Baldari, et al., 1987. EMBO J 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In general, “CRISPR system” refers collectively to transcripts and other elements involved in the expression of or directing the activity of CRISPR-associated (“Cas”) genes, including sequences encoding a Cas gene (e.g., including Cas9 fusions as described herein), a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In some embodiments, CRISPR systems and complexes include one or more nucleic acid repair proteins described herein. In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises a regulatory element operably linked to an enzyme-coding sequence encoding a CRISPR enzyme, such as a Cas protein (e.g., a Cas fusion protein as described herein). Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, including fusions with a nucleic acid repair enzyme as described herein. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some preferred embodiments, the Cas9 protein used in a system or fusion protein of the present invention shares at least 80%, 90%, 95%, or 98% sequence identity with a wildtype S. pyogenes, S. pneumoniae, or S. thermophilus Cas9 reference sequence.

In some embodiments, an enzyme coding sequence encoding a CRISPR enzyme or DNA repair protein is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 1) where NNNNNNNNNNNNXGG (SEQ ID NO: 2) (N is A, G, T, or C; and X can be anything) has a single occurrence in the genome. A unique target sequence in a genome may include an S. pyogenes Cas9 target site of the form MMMMMMMMMNNNNNNNNNNNXGG (SEQ ID NO: 3) where NNNNNNNNNNNXGG (SEQ ID NO: 4) (N is A, G, T, or C; and X can be anything) has a single occurrence in the genome. For the S. thermophilus CRISPR1 Cas9, a unique target sequence in a genome may include a Cas9 target site of the form MMMMMMMMNNNNNNNNNNNNXXAGAAW (SEQ ID NO: 5) where NNNNNNNNNNNNXXAGAAW(SEQ ID NO: 6) (N is A, G, T, or C; X can be anything; and W is A or T) has a single occurrence in the genome. A unique target sequence in a genome may include an S. thermophilus CRISPR1 Cas9 target site of the form MMMMMMMMMNNNNNNNNNNNXXAGAAW (SEQ ID NO: 7) where NNNNNNNNNNNXXAGAAW(SEQID NO: 8) (N is A, G, T, or C; X can be anything; and W is A or T) has a single occurrence in the genome. For the S. pyogenes Cas9, a unique target sequence in a genome may include a Cas9 target site of the form MMMMMMMMNNNNNNNNNNNNXGGXG (SEQ ID NO: 9) where NNNNNNNNNNNNXGGXG (SEQ ID NO: 10) (N is A, G, T, or C; and X can be anything) has a single occurrence in the genome. A unique target sequence in a genome may include an S. pyogenes Cas9 target site of the form MMMMMMMMMNNNNNNNNNNNXGGXG (SEQ ID NO: 11) where NNNNNNNNNNNXGGXG (SEQ ID NO: 12) (N is A, G, T, or C; and X can be anything) has a single occurrence in the genome. In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080; incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. Example illustrations of optimal alignment between a tracr sequence and a tracr mate sequence are provided in U.S. Pat. No. 8,697,359, incorporated herein by reference in its entirety. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 13)
NNNNNNNNNNNNNNNNNNNNgtttttgtactctcaagatttaGA
AAtaaatcttgcagaagctacaaagataaggcttcatgccgaaa
tcaacaccctgtcattttatggcagggtgttttcgttatttaaT
TTTTT;
(2)
(SEQ ID NO: 14)
NNNNNNNNNNNNNNNNNNNNgtttttgtactctcaGAAAtgcag
aagctacaaagataaggcttcatgccgaaatcaacaccctgtca
ttttatggcagggtgttttcgttatttaaTTTTTT;
(3)
(SEQ ID NO: 15)
NNNNNNNNNNNNNNNNNNNNgtttttgtactctcaGAAAtgcag
aagctacaaagataaggcttcatgccgaaatcaacaccctgtca
ttttatggcagggtgtTTTTTT;
(4)
(SEQ ID NO: 16)
NNNNNNNNNNNNNNNNNNNNgtttttgtactctcaGAAAtagca
Agttaaaataaggctagtccgttatcaacttgaaaaagtggcac
cgagtcggtgcTTTTTT;
(5)
(SEQ ID NO: 17)
NNNNNNNNNNNNNNNNNNNNgttttagagctaGAAATAGcaagt
taaaataaggctagtccgttatcaacttgaaaaagtgTTTTTTT;
And
(6)
(SEQ ID NO: 18)
NNNNNNNNNNNNNNNNNNNNgttttagagctagAAATAGcaagt
taaaataaggctagtccgctatcaTTTTTTTT.

In some embodiments, for example, sequences (1) to (3) are used in combination with Cas9 protein or fusion protein of the present invention derived from S. thermophilus CRISPR1. In some embodiments, for example, sequences (4) to (6) are used in combination with Cas9 protein or fusion protein of the present invention derived from S. pyogenes. In some embodiments, the tracr sequence is a separate transcript from a transcript comprising the tracr mate sequence. In some embodiments, CRISPR systems utilize single guide RNAs (sgRNAs) comprising both a tracr and guide sequence.

In some embodiments, provided herein are compositions and methods for enhancing CRISPR gene editing. For example, as described in Example 1 below, during the development of embodiments of the present disclosure, it was determined that fusion proteins comprising a Cas9 polypeptide fused to a DNA repair protein enhanced gene editing (e.g., (HDR).

As used herein, the term “nucleic acid repair protein” refers to any protein (e.g., enzyme, co-factor, inhibitor, enhancer, etc.) involved in nucleic acid (e.g., DNA) repair, which is a collection of processes by which a cell identifies and corrects damage to the DNA molecules that encode its genome. In some embodiments, the nucleic acid repair protein, for example, a homologous recombination protein, a chromatin remodeling protein, replicative polymerase (e.g., a DNA polymerase or an RNA polymerase) or a DNA replication factor.

Specific examples of nucleic acid repair proteins useful in the compositions and methods of the present invention include but are not limited to, a DNA polymerase delta (e.g., DNA polymerase delta III (POLD3; GenBank Accession #NM_006591; NP_006582.1)), POLN (GenBank Accession #NM_181808; NP_861524.2), rfc4 (GenBank Accession #CR536561; CAG38798.1), rfc5 (GenBank Accession #CR407651; CR407651), POLR2H (GenBank Accession #KJ897356; AIC54925.1) PAPD7 (GenBank Accession #KC424495; AGE92663.1) or SIRT6 (GenBank Accession #NM_016539). In some preferred embodiments, the nucleic acid repair protein used in a system or fusion protein of the present invention (e.g., in a system or fusion protein including a Cas9 protein as described above), or portion of domain thereof, share at least 80%, 90%, 95%, or 98% sequence identity with a wild-type nucleic acid repair protein reference sequence (e.g., POLD3 (GenBank Accession #NM_006591; NP_006582.1), POLN (GenBank Accession #NM_181808; NP_861524.2), rfc4 (GenBank Accession #CR536561; CAG38798.1), rfc5 (GenBank Accession #CR407651; CR407651), POLR2H (GenBank Accession #KJ897356; AIC54925.1) PAPD7 (GenBank Accession #KC424495; AGE92663.1) or SIRT6 (GenBank Accession #NM_016539).

In some embodiments, a portion of the nucleic acid repair protein is utilized (e.g., functional domain, fragment, etc.). In some embodiments, provided herein is a composition, comprising: a nucleic acid encoding a fusion protein comprising a Cas9 polypeptide fused to a nucleic acid repair protein and/or the corresponding fusion protein.

In some embodiments, rather than a fusion protein, the Cas9 polypeptide and the nucleic acid repair protein are provided as separate polypeptides or nucleic acids encoding such polypeptides.

In some embodiments, the nucleic acid is a vector. In some embodiments, the first and second nucleic acid are on the same or different vectors. For example, in some embodiments, the first and second nucleic acid are on the same vector and are separated by an internal ribosome entry site (IRES). Exemplary vectors and expression systems are described herein.

In some embodiments, the CRISPR enzyme is part of a fusion protein comprising one or more nucleic acid repair proteins or domains (e.g. about or more than about 1, 2, 3, 4, 5, 6, 7, 8, 9, 10, or more proteins or domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and nucleic acid repair proteins described herein. A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions.

In an aspect of the invention, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the invention, the DNA molecule encoding the gene product may be introduced into the cell via a vector. In a preferred embodiment of the invention the gene product is luciferase. In a further embodiment of the invention the expression of the gene product is decreased.

In some aspects, the invention provides methods comprising delivering one or more polynucleotides, such as or one or more vectors, systems or filaments as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a CRISPR enzyme in combination with (and optionally complexed with) a guide sequence or filament is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon. TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycations or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995): Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and W² cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is a mammal, such as a mouse, rat, or rabbit. In certain embodiments, the organism or subject is a plant. In certain embodiments, the organism or subject or plant is algae. Methods for producing transgenic plants and animals are known in the art, and generally begin with a method of cell transfection, such as described herein. Transgenic animals are also provided, as are transgenic plants, especially crops and algae. The transgenic animal or plant may be useful in applications outside of providing a disease model. These may include food or feed production through expression of, for instance, higher protein, carbohydrate, nutrient or vitamins levels than would normally be seen in the wildtype. In this regard, transgenic plants, especially pulses and tubers, and animals, especially mammals such as livestock (cows, sheep, goats and pigs), but also poultry and edible insects, are preferred.

Transgenic algae or other plants such as rape may be particularly useful in the production of vegetable oils or biofuels such as alcohols (especially methanol and ethanol), for instance. These may be engineered to express or overexpress high levels of oil or alcohols for use in the oil or biofuel industries.

In some embodiments, the nucleic acids, fusion proteins, or polypeptides described herein are provided in the form of a kit or system that comprises a plurality (e.g., one or two) of guide RNAs (e.g., sgRNAs) and optionally a nucleic acid encoding an exogenous gene of interest (e.g., on a vector). Exemplary genes of interest are described herein.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

Certain embodiments provide a method (e.g., gene editing method), comprising: introducing a system described herein into a cell. In some embodiments, the introducing results in disruption, deletion, or insertion of a target nucleic acid (e.g., gene) in the cell. In some embodiments, the gene editing results in an increase or decrease in expression of an endogenous or exogenous gene in the cell. In some embodiments, the cell is a eukaryotic cell (e.g., a mammalian (e.g., human) cell). In some embodiments, the cell is in vitro, ex vivo, or in vivo. In some embodiments, the method treats a disease or condition in a subject.

For example, in one aspect, the invention provides for methods of modifying a target polynucleotide in a eukaryotic cell, which may be in vivo, ex vivo or in vitro. In some embodiments, the method comprises sampling a cell or population of cells from a human or non-human animal or plant (including micro-algae), and modifying the cell or cells. Culturing may occur at any stage ex vivo. The cell or cells may even be re-introduced into the non-human animal or plant (including micro-algae).

In one aspect, the invention provides for methods of modifying a target polynucleotide in a eukaryotic cell. In some embodiments, the method comprises allowing a CRISPR complex to bind to the target polynucleotide to effect cleavage of the target polynucleotide thereby modifying the target polynucleotide, wherein the CRISPR complex comprises a CRISPR enzyme complexed with a guide sequence hybridized to a target sequence within the target polynucleotide, wherein the guide sequence is linked to a tracr mate sequence which in turn hybridizes to a tracr sequence or is an sgRNA.

In one aspect, the invention provides a method of modifying expression of a polynucleotide in a eukaryotic cell. In some embodiments, the method comprises allowing a CRISPR complex to bind to the polynucleotide such that the binding results in increased or decreased expression of the polynucleotide; wherein the CRISPR complex comprises a CRISPR enzyme complexed with a guide sequence hybridized to a target sequence within the polynucleotide, wherein the guide sequence is linked to a tracr mate sequence which in turn hybridizes to a tracr sequence. In another aspect, the invention provides a method of modifying expression of a polynucleotide in a eukaryotic cell.

With recent advances in crop genomics, the ability to use CRISPR-Cas systems to perform efficient and cost effective gene editing and manipulation will allow the rapid selection and comparison of single and multiplexed genetic manipulations to transform such genomes for improved production and enhanced traits. In this regard reference is made to U.S. patents and publications: U.S. Pat. No. 6,603,061-Agrobacterium-Mediated Plant Transformation Method; U.S. Pat. No. 7,868,149-Plant Genome Sequences and Uses Thereof and US 2009/0100536-Transgenic Plants with Enhanced Agronomic Traits, all the contents and disclosure of each of which are herein incorporated by reference in their entirety. In the practice of the invention, the contents and disclosure of Morrell et al “Crop genomics:advances and applications” Nat Rev Genet. 2011 Dec. 29; 13(2):85-96 are also herein incorporated by reference in their entirety. In plants, pathogens are often host-specific. For example, Fusarium oxysporum f. sp. lycopersici causes tomato wilt but attacks only tomato, and F. oxysporn f. dianthii Puccinia graminis f. sp. tritici attacks only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield, Quality, Uniformity, Hardiness, Resistance. The sources of resistance genes include native or foreign Varieties, Heirloom Varieties, Wild Plant Relatives, and Induced Mutations, e.g., treating plant material with mutagenic agents. Using the present invention, plant breeders are provided with a new tool to induce mutations. Accordingly, one skilled in the art can analyze the genome of sources of resistance genes, and in Varieties having desired characteristics or traits employ the present invention to induce the rise of resistance genes, with more precision than previous mutagenic agents and hence accelerate and improve plant breeding programs.

In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types. As such the CRISPR complex of the invention has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide. The guide sequence is linked to a tracr mate sequence, which in turn hybridizes to a tracr sequence.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence) Examples of PAM sequences are given in the examples section below, and the skilled person will be able to identify further PAM sequences for use with a given CRISPR enzyme.

The target polynucleotide of a CRISPR complex may include a number of disease-associated genes and polynucleotides as well as signaling biochemical pathway-associated genes and polynucleotides as listed in U.S. provisional patent applications 61/736,527 and 61/748,427, both entitled SYSTEMS METHODS AND COMPOSITIONS FOR SEQUENCE MANIPULATION filed on Dec. 12, 2012 and Jan. 2, 2013, respectively, the contents of all of which are herein incorporated by reference in their entirety.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web.

Examples of disease-associated genes and polynucleotides are listed in Tables A and B. Disease specific information is available from McKusick-Nathans Institute of Genetic Medicine, Johns Hopkins University (Baltimore, Md.) and National Center for Biotechnology Information, National Library of Medicine (Bethesda, Md.), available on the World Wide Web. Examples of signaling biochemical pathway-associated genes and polynucleotides are listed in Table C.

Mutations in these genes and pathways can result in production of improper proteins or proteins in improper amounts which affect function. genes, proteins and pathways may be the target polynucleotide of a CRISPR complex.

| DISEASE | |
|---|---|
| DISORDERS | GENE(S) |
| Neoplasia | PTEN; ATM; ATR; EGFR; ERBB2; ERBB3; |
| | ERBB4; Notch1; Notch2; Notch3; Notch4; AKT; |
| | AKT2; AKT3; HIF; HIF1a; HIF3a; Met; HRG; Bcl2; |
| | PPAR alpha; PPAR gamma; WT1 (Wilms Tumor); |
| | FGF Receptor Family members (5 members: 1, 2, 3, 4, |
| | 5); CDKN2a; APC; RB (retinoblastoma); MEN1; |
| | VHL; BRCA1; BRCA2; AR (Androgen Receptor); |
| | TSG101; IGF; IGF Receptor; Igf1 (4 variants); Igf2 (3 |
| | variants); Igf 1 Receptor; Igf 2 Receptor; Bax; Bcl2; |
| | caspases family (9 members: 1, 2, 3, 4, 6, 7, 8, 9, 12); |
| | Kras; Apc |
| Age-related | Aber; Ccl2; Cc2; cp (ceruloplasmin); Timp3; |
| Macular | cathepsinD; Vldlr; Ccr2 |
| Degeneration | |
| Schizophrenia | Neuregulin1 (Nrg1); Erb4 (receptor for Neuregulin); |
| | Complexin1 (Cplx1); Tph1 Tryptophan hydroxylase; |
| | Tph2 Tryptophan hydroxylase 2; Neurexin 1; GSK3; |
| | GSK3a; GSK3b |
| Disorders | 5-HTT (Slc6a4); COMT; DRD (Drd1a); SLC6A3; |
| | DAOA; DTNBP1; Dao (Dao1) |
| Trinucleotide | HTT (Huntington's Dx); SBMA/SMAX1/AR |
| Repeat | (Kennedy's Dx); FXN/X25 (Friedrich's Ataxia); ATX3 |
| Disorders | (Machado-Joseph's Dx); ATXN1 and ATXN2 |
| | (spinocerebellar ataxias); DMPK (myotonic |
| | dystrophy); Atrophin-1 and Atn 1 (DRPLA Dx); CBP |
| | (Creb-BP-global instability); VLDLR (Alzheimer's); |
| | Atxn7; Atxn10 |
| Fragile X | FMR2; FXR1; FXR2; mGLUR5 |
| Syndrome | |
| Secretase | APH-1 (alpha and beta); Presenilin (Psen1); nicastrin |
| Related. | (Ncstn); PEN-2 |
| Disorders | |
| Others | Nos1; Parp1; Nat1; Nat2 |
| Prion-related | Prp |
| disorders | |
| ALS | SOD1; ALS2; STEX; FUS; TARDBP; VEGF |
| | (VEGF-a; VEGF-b; VEGF-c) |
| Drug | Prkce (alcohol); Drd2; Drd4; ABAT (alcohol); |
| addiction | GRIA2; Grm5; Grin1; Htr1b; Grin2a; Drd3; Pdyn; |
| | Grial (alcohol) |
| Autism | Mecp2; BZRAP1; MDGA2; Sema5A; Neurexin 1; |
| | Fragile X (FMR2 (AFF2); FXR1; FXR2; Mglur5) |
| Alzheimer's | E1; CHIP; UCH; UBB; Tau; LRP; PICALM; |
| Disease | Clusterin; PS1; SORL1; CR1; Vld1r; Uba1; Uba3; |
| | CHIP28 (Aqp1, Aquaporin 1); Uchl1; Uchl3; APP |
| Inflammation | 1L-10; IL-1 (1L-1a; IL-1b); 1L-13; IL-17 (IL-17a |
| | (CTLA8); IL-17b; IL-17c; IL-17d; IL-17f); II-23; |
| | Cx3er1; ptpn22; TNFa; NOD2/CARD15 for IBD; IL- |
| | 6; 1L-12 (1L-12a; 1L-12b); CTLA4; Cx3cl1 |
| Parkinson's | x-Synuclein; DJ-1; LRRK2; Parkin; PINK1 |
| Disease | |

TABLE B

| | |
|---|---|
| Blood and | Anemia (CDAN1, CDA1, RPS19, DBA, PKLR, |
| coagulation | PK1, NT5C3, UMPH1, PSN1, RHAG, RH50A, |
| diseases | |
| and disorders | NRAMP2, SPTB, ALAS2, ANH1, ASB, ABCB7, |
| | ABC7, ASAT); Bare lymphocyte syndrome |
| | (TAPBP, TPSN, TAP2, ABCB3, PSF2, RING11, |
| | MHC2TA, C2TA, RFX5, RFXAP, RFX5), |
| | Bleeding disorders (TBXA2R, P2RX1, P2X1); |
| | Factor H and factor H-like 1 (HF1, CFH, HUS); |
| | Factor V and factor VIII (MCFD2); Factor VII |
| | deficiency (F7); Factor X deficiency (F10); Factor |
| | XI deficiency (F11); Factor XII deficiency (F12, |
| | HAF); Factor XIIIA deficiency (F13A1, F13A); |
| | Factor XIIIB deficiency (F13B); Fanconi anemia |
| | (FANCA, FACA, FA1, FA, FAA, FAAP95, |
| | FAAP90, FLJ34064, FANCB, FANCC, FACC, |
| | BRCA2, FANCD1, FANCD2, FANCD, FACD, |
| | FAD, FANCE, FACE, FANCF, XRCC9, FANCG, |
| | BRIP1, BACH1, FANCJ, PHF9, FANCL, FANCM, |
| | KIAA1596); Hemophagocytic lymphohistiocytosis |
| | disorders (PRF1, HPLH2, UNC13D, MUNC13-4, |
| | HPLH3, HLH3, FHL3); Hemophilia A (F8, F8C, |
| | HEMA); Hemophilia B (F9, HEMB), Hemorrhagic |
| | disorders (PI, ATT, F5); Leukocyde deficiencies |
| | and disorders (ITGB2, CD18, LCAMB, LAD, |
| | EIF2B1, EIF2BA, EIF2B2, EIF2B3, EIF2B5, |
| | LVWM, CACH, CLE, EIF2B4); Sickle cell anemia |
| | (HBB); Thalassemia (HBA2, HBB, HBD, LCRB, |
| | HBA1). |
| Cell | B-cell non-Hodgkin lymphoma (BCL7A, BCL7); |
| dysregulation | |
| and oncology | Leukemia (TAL1 TCL5, SCL, TAL2, FLT3, NBS1, |
| diseases and | NBS, ZNFN1A1, IK1, LYF1, HOXD4, HOX4B, |
| disorders | BCR, CML, PHL, ALL, ARNT, KRAS2, RASK2, |
| | GMPS, AF10, ARHGEF12, LARG, KIAA0382, |
| | CALM, CLTH, CEBPA, CEBP, CHIC2, BTL, |
| | FLT3, KIT, PBT, LPP, NPM1, NUP214, D9S46E, |
| | CAN, CAIN, RUNX1, CBFA2, AML1, WHSC1L1, |
| | NSD3, FLT3, AF1Q, NPM1, NUMA1, ZNF145, |
| | PLZF, PML, MYL, STAT5B, AF10, CALM, |
| | CLTH, ARL11, ARLTS1, P2RX7, P2X7, BCR, |
| | CML, PHL, ALL, GRAF, NF1, VRNF, WSS, |
| | NFNS, PTPN11, PTP2C, SHP2, NS1, BCL2, |
| | CCND1, PRAD1, BCL1, TCRA, GATA1, GF1, |
| | ERYF1, NFE1, ABL1, NQO1, DIA4, NMOR1, |
| | NUP214, DOS46E, CAN, CAIN). |
| Inflammation | AIDS (KIR3DL1, NKAT3, NKB1, AMB11, |
| and | |
| immune | KIR3DS1, IFNG, CXCL12, SDF1); Autoimmune |
| related | |
| diseases and | lymphoproliferative syndrome (TNFRSF6, APT1, |
| disorders | FAS, CD95, ALPS1A); Combined immuno- |
| | deficiency, (IL2RG, SCIDX1, SCIDX, IMD4); |
| | HIV-1 (CCL5, SCYA5, D17S136E, TCP228), HIV |
| | susceptibility or infection (IL10, CSIF, CMKBR2, |
| | CCR2, CMKBR5, CCCKR5 (CCR5)); Immuno- |
| | deficiencies (CD3E, CD3G, AICDA, AID, HIGM2, |
| | TNFRSF5, CD40, UNG, DGU, HIGM4, TNFSF5, |
| | CD40LG, HIGM1, IGM, FOXP3, IPEX, AIID, |
| | XPID, PIDX, TNFRSF14B, TACI); Inflammation |
| | (IL-10, IL-1 (IL-1a, IL-1b), IL-13, IL-17 (IL-17a |
| | (CTLA8), IL-17b, IL-17c, IL-17d, IL-17f, II-23, |
| | Cx3cr1, ptpn22, TNFa, NOD2/CARD15 for IBD, |
| | IL-6, IL-12 (IL-12a, IL-12b), CTLA4, Cx3cl1); |
| | Severe combined immunodeficiencies (SCIDs) |
| | (JAK3, JAKL, DCLRE1C, ARTEMIS, SCIDA, |
| | RAG1, RAG2, ADA, PTPRC, CD45, LCA, IL7R, |
| | CD3D, T3D, IL2RG, SCIDX1, SCIDX, IMD4). |
| Metabolic, | Amyloid neuropathy (TTR, PALB); Amyloidosis |
| liver, | |
| kidney and | (APOA1, APP, AAA, CVAP, AD1, GSN, FGA, |
| protein | |
| diseases and | LYZ, TTR, PALB); Cirrhosis (KRT18, KRT8, |
| disorders | CIRH1A, NAIC, TEX292, KIAA1988); Cystic |
| | fibrosis (CFTR, ABCC7, CF, MRP7); Glycogen |
| | storage diseases (SLC2A2, GLUT2, G6PC, G6PT, |
| | G6PT1, GAA, LAMP2, LAMPB, AGL, GDE, |
| | GBE1, GYS2, PYGL, PFKM); Hepatic adenoma, |
| | 142330 (TCF1, HNF1A, MODY3), Hepatic failure, |
| | early onset, and neurologic disorder (SCOD1, |
| | SCO1), Hepatic lipase deficiency (LIPC), Hepato- |
| | blastoma, cancer and carcinomas (CTNNB1, |
| | PDGFRL, PDGRL, PRLTS, AXIN1, AXIN, |
| | CTNNB1, TP53, P53, LFS1, IGF2R, MPRI, MET, |
| | CASP8, MCH5; Medullary cystic kidney disease |
| | (UMOD, HNFJ, FJHN, MCKD2, ADMCKD2); |
| | Phenylketonuria (PAH, PKU1, QDPR, DHPR, |
| | PTS); Polycystic kidney and hepatic disease |
| | (FCYT, PKHD1, ARPKD, PKD1, PKD2, PKD4, |
| | PKDTS, PRKCSH, G19P1, PCLD, SEC63). |
| Muscular/ | Becker muscular dystrophy (DMD, BMD, MYF6), |
| Skeletal | |
| diseases and | Duchenne Muscular Dystrophy (DMD, BMD); |
| disorders | Emery-Dreifuss muscular dystrophy (LMNA, |
| | LMN1, EMD2, FPLD, CMD1A, HGPS, LGMD1B, |
| | LMNA, LMN1, EMD2, FPLD, CMD1A); Facio- |
| | scapulohumeral muscular dystrophy (FSHMD1A, |
| | FSHD1A); Muscular dystrophy (FKRP, MDC1C, |
| | LGMD2I, LAMA2, LAMM, LARGE, KIAA0609, |
| | MDC1D, FCMD, TTID, MYOT, CAPN3, CANP3, |
| | DYSF, LGMD2B, SGCG, LGMD2C, DMDA1, |
| | SCG3, SGCA, ADL, DAG2, LGMD2D, DMDA2, |
| | SGCB, LGMD2E, SGCD, SGD, LGMD2F, |
| | CMD1L, TCAP, LGMD2G, CMD1N, TRIM32, |
| | HT2A, LGMD2H, FKRP, MDC1C, LGMD2I, |
| | TTN, CMD1G, TMD, LGMD2J, POMT1, CAV3, |
| | LGMD1C, SEPN1, SELN, RSMD1, PLEC1, |
| | PLTN, EBS1); Osteopetrosis (LRP5, BMND1, |
| | LRP7, LR3, OPPG, VBCH2, CLCN7, CLC7, |
| | OPTA2, OSTM1, GL, TCIRG1, TIRC7, OC116, |
| | OPTB1); Muscular atrophy (VAPB, VAPC, ALS8, |
| | SMN1, SMA1, SMA2, SMA3, SMA4, BSCL2, |
| | SPG17, GARS, SMAD1, CMT2D, HEXB, |
| | IGHMBP2, SMUBP2, CATF1, SMARD1). |
| Neurological | ALS (SOD1, ALS2, STEX, FUS, TARDBP, VEGF |
| and | |
| neuronal | (VEGF-a, VEGF-b, VEGF-c); Alzheimer disease |
| diseases | |
| and disorders | (APP, AAA, CVAP, AD1, APOE, AD2, PSEN2, |
| | AD4, STM2, APBB2, FE65L1, NOS3, PLAU, |
| | URK, ACE, DCP1, ACE1, MPO, PACIP1, |
| | PAXIP1L, PTIP, A2M, BLMH, BMH, PSEN1, |
| | AD3); Autism (Mecp2, BZRAP1, MDGA2, |
| | Sema5A, Neurexin 1, GLO1, MECP2, RTT, |
| | PPMX, MRX16, MRX79, NLGN3, NLGN4, |
| | KIAA1260, AUTSX2); Fragile X Syndrome |
| | (FMR2, FXR1, FXR2, mGLUR5); Huntington's |
| | disease and disease like disorders (HD, IT15, |
| | PRNP, PRIP, JPH3, JP3, HDL2, TBP, SCA17); |
| | Parkinson disease (NR4A2, NURR1, NOT, TINUR, |
| | SNCAIP, TBP, SCA17, SNCA, NACP, PARK1, |
| | PARK4, DJ1, PARK7, LRRK2, PARK8, PINK1, |
| | PARK6, UCHL1, PARK5, SNCA, NACP, PARK1, |
| | PARK4, PRKN, PARK2, PDJ, DBH, NDUFV2); |
| | Rett syndrome (MECP2, RTT, PPMX, MRX16, |
| | MRX79, CDKL5, STK9, MECP2, RTT, PPMX, |
| | MRX16, MRX79, x-Synuclein, DJ-1); Schizo- |
| | phrenia (Neuregulin1 (Nrg1), Erb4 (receptor for |
| | Neuregulin), Complexin1 (Cplx1), Tph1 Trypto- |
| | phan hydroxylase, Tph2, Tryptophan hydroxylase 2, |
| | Neurexin 1, GSK3, GSK3a, GSK3b, 5-HTT |
| | (Slc6a4), COMT, DRD (Drd1a), SLC6A3, DAOA, |
| | DTNBP1, Dao (Dao1)); Secretase Related Dis- |
| | orders (APH-1 (alpha and beta), Presenilin (Psen1), |
| | nicastrin, (Ncstn), PEN-2, Nos1, Parp1, Nat1, |
| | Nat2); Trinucleotide Repeat Disorders (HTT |
| | (Huntington's Dx), SBMA/SMAX1/AR (Kennedy's |
| | Dx), FXN/X25 (Friedrich's Ataxia), ATX3 |
| | (Machado- Joseph's Dx), ATXN1 and ATXN2 |
| | (spinocerebellar ataxias), DMPK (myotonic |
| | dystrophy), Atrophin-1 and Atn1 (DRPLA Dx), |
| | CBP (Creb-BP - global instability), VLDLR |
| | (Alzheimer's), Atxn7, Atxn10). |
| Occular | Age-related macular degeneration (Aber, Ccl2, Cc2, |
| diseases | |
| and disorders | cp (ceruloplasmin), Timp3, cathepsinD, Vldlr, |
| | Ccr2); Cataract (CRYAA, CRYA1, CRYBB2, |
| | CRYB2, PITX3, BFSP2, CP49, CP47, CRYAA, |
| | CRYA1, PAX6, AN2, MGDA, CRYBA1, CRYB1, |
| | CRYGC, CRYG3, CCL, LIM2, MP19, CRYGD, |
| | CRYG4, BFSP2, CP49, CP47, HSF4, CTM, HSF4, |
| | CTM, MIP, AQP0, CRYAB, CRYA2, CTPP2, |
| | CRYBB1, CRYGD, CRYG4, CRYBB2, CRYB2, |
| | CRYGC, CRYG3, CCL, CRYAA, CRYA1, GJA8, |
| | CX50, CAE1, GJA3, CX46, CZP3, CAE3, CCM1, |
| | CAM, KRIT1); Corneal clouding and dystrophy |
| | (APOA1, TGFBI, CSD2, CDGG1, CSD, BIGH3, |
| | CDG2, TACSTD2, TROP2, M1S1, VSX1, RINX, |
| | PPCD, PPD, KTCN, COL8A2, FECD, PPCD2, |
| | PIP5K3, CFD); Cornea plana congenital (KERA, |
| | CNA2); Glaucoma (MYOC, TIGR, GLC1A, JOAG, |
| | GPOA, OPTN, GLC1E, FIP2, HYPL, NRP, |
| | CYP1B1, GLC3A, OPA1, NTG, NPG, CYP1B1, |
| | GLC3A); Leber congenital amaurosis (CRB1, |
| | RP12, CRX, CORD2, CRD, RPGRIP1, LCA6, |
| | CORD9, RPE65, RP20, AIPL1, LCA4, GUCY2D, |
| | GUC2D, LCA1, CORD6, RDH12, LCA3); |
| | Macular dystrophy (ELOVL4, ADMD, STGD2, |
| | STGD3, RDS, RP7, PRPH2, PRPH, AVMD, |
| | AOFMD, VMD2). |
| Epilepsy, | EPM2A, MELF, EPM2 |
| myoclonic, | |
| Lafora type, | |
| 254780 | |
| Epilepsy, | NHLRC1, EPM2A, EPM2B |
| myoclonic, | |
| Lafora type, | |
| 254780 | |
| Duchenne | DMD, BMD |
| muscular | |
| dystrophy, | |
| 310200 (3) | |
| AIDS, | KIR3DL1, NKAT3, NKB1, AMB11, KIR3DS1 |
| delayed/rapid | |
| progression | |
| to (3) | |
| AIDS, rapid | IFNG |
| progression | |
| to, | |
| 609423 (3) | |
| AIDS, | CXCL12, SDF1 |
| resistance to | |
| (3) | |
| Alpha 1- | SERPINA1 [serpin peptidase inhibitor, clade A |
| Antitrypsin | |
| Deficiency | (alpha-1 antiproteinase, antitrypsin), member 1]; |
| | SERPINA2 [serpin peptidase inhibitor, clade A |
| | (alpha-1 antiproteinase, antitrypsin), member 2]; |
| | SERPINA3 [serpin peptidase inhibitor, clade A |
| | (alpha-1 antiproteinase, antitrypsin), member 3]; |
| | SERPINA5 [serpin peptidase inhibitor, clade A |
| | (alpha-1 antiproteinase, antitrypsin), member 5]; |
| | SERPINA6 [serpin peptidase inhibitor, clade A |
| | (alpha-1 antiproteinase, antitrypsin), member 6]; |
| | SERPINA7 [serpin peptidase inhibitor, clade A |
| | (alpha-1 antiproteinase, antitrypsin), member 7]; ” |
| | AND “SERPLNA6 (serpin peptidase inhibitor, |
| | clade A (alpha-1 antiproteinase, antitrypsin), |
| | member 6) |

TABLE C

| CELLULAR | |
|---|---|
| FUNCTION | GENES |
| PI3K/AKT | PRKCE; ITGAM; ITGA5; IRAK1; PRKAA2; |
| Signaling | |
| | EIF2AK2; PTEN; EIF4E; PRKCZ; GRK6; |
| | MAPK1; TSC1; PLK1; AKT2; IKBKB; |
| | P1K3CA; CDK8; CDKN1B; NFKB2; BCL2; |
| | PIK3CB; PPP2R1A; MAPK8; BCL2L1; |
| | MAPK3; TSC2; ITGA1; KRAS; EIF4EBP1; |
| | RELA; PRKCD; NOS3; PRKAA1; MAPK9; |
| | CDK2; PPP2CA; PIM1; ITGB7; YWHAZ; ILK; |
| | TP53; RAF1.; IKBKG; RELB; DYRK1A; |
| | CDKN1A; ITGB1; MAP2K2; JAK1; AKT1; |
| | JAK2; PIK3R1; CHUK; PDPK1; PPP2R5C; |
| | CTNNB1.; MAP2K1; NFKB1; PAK3; ITGB3; |
| | CCND1; GSK3A; FRAP1; SFN; ITGA2; TTK; |
| | CSNK1A1; BRAF; GSK3B; AKT3; FOXO1; |
| | SGK; HSP90AA1; RPS6KB1 |
| ERK/MAPK | PRKCE; ITGAM; ITGA5; HSPB1; IRAK1; |
| Signaling | |
| | PRKAA2; EIF2AK2; RAC1; RAP1A; TLN1; |
| | EIF4E; ELK1; GRK6; MAPK1; RAC2; PLK1; |
| | AKT2; P1K3CA; CDK8; CREB1; PRKCI; |
| | PTK2; FOS; RPS6KA4; PIK3CB; PPP2R1A; |
| | PIK3C3; MAPK8; MAPK3; ITGA1; ETS1; |
| | KRAS; MYCN; EIF4EBP1; PPARG; PRKCD; |
| | PRKAA1; MAPK9; SRC; CDK2; PPP2CA; |
| | PIM1; PIK3C2A; ITGB7; YWHAZ; PPP1CC; |
| | KSR1; PXN; RAF1; FYN; DYRK1A; ITGB1; |
| | MAP2K2; PAK4; PIK3R1; STAT3; PPP2R5C; |
| | MAP2K1; PAK3; ITGB3; ESR1; ITGA2; MYC; |
| | TTK; CSNK1A1; CRKL; BRAF; ATF4; |
| | PRKCA; SRF; STAT1; SGK |
| Glucocorticoid | RAC1; TAF4B; EP300; SMAD2; TRAF6; |
| Receptor | |
| Signaling | PCAF; ELK1; MAPK1; SMAD3; AKT2; |
| | IKBKB; NCOR2; UBE2I; P1K3CA; CREB1; |
| | FOS; HSPA5; NFKB2; BCL2; MAP3K14; |
| | STAT5B; PIK3CB; PIK3C3; MAPK8; BCL2L1; |
| | MAPK3; TSC22D3; MAPK10; NRIP1; KRAS; |
| | MAPK13; RELA; STAT5A; MAPK9; NOS2A; |
| | PBX1; NR3C1; PIK3C2A; CDKN1C; TRAF2; |
| | SERPINE1; NCOA3; MAPK14; TNF; RAF1; |
| | IKBKG; MAP3K7; CREBBP; CDKN1A; |
| | MAP2K2; JAK1; IL8; NCOA2; AKT1; JAK2; |
| | PIK3R1; CHUK; STAT3; MAP2K1; NFKB1; |
| | TGFBR1; ESR1; SMAD4; CEBPB; JUN; AR; |
| | AKT3; CCL2; MMP1; STAT1; IL6; HSP90AA1 |
| Axonal Guidance | PRKCE; ITGAM; ROCK1; ITGA5; CXCR4; |
| Signaling | ADAM12; IGF1; RAC1; RAP1A; E1F4E; |
| | PRKCZ; NRP1; NTRK2; ARHGEF7; SMO; |
| | ROCK2; MAPK1; PGF; RAC2; PTPN11; |
| | GNAS; AKT2; P1K3CA; ERBB2; PRKC1; |
| | PTK2; CFL1; GNAQ; PIK3CB; CXCL12; |
| | PIK3C3; WNT11; PRKD1; GNB2L1; ABL1; |
| | MAPK3; ITGA1; KRAS; RHOA; PRKCD; |
| | PIK3C2A; ITGB7; GLI2; PXN; VASP; RAF1; |
| | FYN; ITGB1; MAP2K2; PAK4; ADAM17; |
| | AKT1; PIK3R1; GLI1; WNT5A; ADAM10; |
| | MAP2K1; PAK3; ITGB3; CDC42; VEGFA; |
| | ITGA2; EPHA8; CRKL; RND1; GSK3B; |
| | AKT3; PRKCA |
| Ephrin Receptor | PRKCE; ITGAM; ROCK1; ITGA5; CXCR4; |
| Signaling | IRAK1; PRKAA2; EIF2AK2; RAC1; RAP1A; |
| | GRK6; ROCK2; MAPK1; PGF; RAC2; |
| | PTPN11; GNAS; PLK1; AKT2; DOK1; CDK8; |
| | CREB1; PTK2; CFL1; GNAQ; MAP3K14; |
| | CXCL12; MAPK8; GNB2L1; ABL1; MAPK3; |
| | ITGA1; KRAS; RHOA; PRKCD; PRKAA1; |
| | MAPK9; SRC; CDK2; PIM1; ITGB7; PXN; |
| | RAF1; FYN; DYRK1A; ITGB1; MAP2K2; |
| | PAK4, AKT1; JAK2; STAT3; ADAM10; |
| | MAP2K1; PAK3; ITGB3; CDC42; VEGFA; |
| | ITGA2; EPHA8; TTK; CSNK1A1; CRKL; |
| | BRAF; PTPN13; ATF4; AKT3; SGK |
| Actin Cytoskeleton | ACTN4; PRKCE; ITGAM; ROCK1; ITGA5; |
| Signaling | IRAK1; PRKAA2; EIF2AK2; RAC1; INS; |
| | ARHGEF7; GRK6; ROCK2; MAPK1; RAC2; |
| | PLK1; AKT2; P1K3CA; CDK8; PTK2; CFL1; |
| | PIK3CB; MYH9; DIAPH1; PIK3C3; MAPK8; |
| | F2R; MAPK3; SLC9A1; ITGA1; KRAS; RHOA; |
| | PRKCD; PRKAA1; MAPK9; CDK2; PIM1; |
| | PIK3C2A; ITGB7; PPP1CC; PXN; VIL2; RAF1; |
| | GSN; DYRK1A; ITGB1; MAP2K2; PAK4; |
| | PIP5K1A; PIK3R1; MAP2K1; PAK3; ITGB3; |
| | CDC42; APC; ITGA2; TTK; CSNK1A1; CRKL; |
| | BRAF; VAV3; SGK |
| Huntington's | PRKCE; IGF1; EP300; RCOR1.; PRKCZ; |
| Disease | |
| Signaling | HDAC4; TGM2; MAPK1; CAPNS1; AKT2; |
| | EGFR; NCOR2; SP1; CAPN2; P1K3CA; |
| | HDAC5; CREB1; PRKC1; HSPA5; REST; |
| | GNAQ; PIK3CB; PIK3C3; MAPK8; IGF1R; |
| | PRKD1; GNB2L1; BCL2L1; CAPN1; MAPK3; |
| | CASP8; HDAC2; HDAC7A; PRKCD; HDAC11; |
| | MAPK9; HDAC9; PIK3C2A; HDAC3; TP53; |
| | CASP9; CREBBP; AKT1; PIK3R1; PDPK1; |
| | CASP1; APAF1; FRAP1; CASP2; JUN; BAX; |
| | ATF4; AKT3; PRKCA; CLTC; SGK; HDAC6; |
| | CASP3 |
| Apoptosis Signaling | PRKCE; ROCK1; BID; IRAK1; PRKAA2; |
| | EIF2AK2; BAK1; BIRC4; GRK6; MAPK1; |
| | CAPNS1; PLK1; AKT2; IKBKB; CAPN2; |
| | CDK8; FAS; NFKB2; BCL2; MAP3K14; |
| | MAPK8; BCL2L1; CAPN1; MAPK3; CASP8; |
| | KRAS; RELA; PRKCD; PRKAA1; MAPK9; |
| | CDK2; PIM1; TP53; TNF; RAF1; IKBKG; |
| | RELB; CASP9; DYRK1A; MAP2K2; CHUK; |
| | APAF1; MAP2K1; NFKB1; PAK3; LMNA; |
| | CASP2; BIRC2; TTK; CSNK1A1; BRAF; BAX; |
| | PRKCA; SGK; CASP3; BIRC3; PARP1 |
| B Cell Receptor | RAC1; PTEN; LYN; ELK1; MAPK1; RAC2; |
| Signaling | PTPN11; AKT2; IKBKB; P1K3CA; CREB1; |
| | SYK; NFKB2; CAMK2A; MAP3K14; PIK3CB; |
| | PIK3C3; MAPK8; BCL2L1; ABL1; MAPK3; |
| | ETS1; KRAS; MAPK13; RELA; PTPN6; |
| | MAPK9; EGR1; PIK3C2A; BTK; MAPK14; |
| | RAF1; IKBKG; RELB; MAP3K7; MAP2K2; |
| | AKT1; PIK3R1; CHUK; MAP2K1; NFKB1; |
| | CDC42; GSK3A; FRAP1; BCL6; BCL10; JUN; |
| | GSK3B; ATF4; AKT3; VAV3; RPS6KB1 |
| Leukocyte | ACTN4; CD44; PRKCE; ITGAM; ROCK1; |
| Extravasation | |
| Signaling | CXCR4; CYBA; RAC1; RAP1A; PRKCZ; |
| | ROCK2; RAC2; PTPN11; MMP14; P1K3CA; |
| | PRKCI; PTK2; PIK3CB; CXCL12; PIK3C3; |
| | MAPK8; PRKD1; ABL1; MAPK10; CYBB; |
| | MAPK13; RHOA; PRKCD; MAPK9; SRC; |
| | PIK3C2A; BTK; MAPK14; NOX1; PXN; VIL2; |
| | VASP; ITGB1; MAP2K2; CTNND1; PIK3R1; |
| | CTNNB1; CLDN1; CDC42; F11R; ITK; CRKL; |
| | VAV3; CTTN; PRKCA; MMP1; MMP9 |
| Integrin Signaling | ACTN4; ITGAM; ROCK1; ITGA5; RAC1; |
| | PTEN; RAP1A; TLN1; ARHGEF7; MAPK1; |
| | RAC2; CAPNS1; AKT2; CAPN2; P1K3CA; |
| | PTK2; PIK3CB; PIK3C3; MAPK8; CAV1; |
| | CAPN1; ABL1; MAPK3; ITGA1; KRAS; |
| | RHOA; SRC; PIK3C2A; ITGB7; PPP1CC; ILK; |
| | PXN; VASP; RAF1; FYN; ITGB1; MAP2K2; |
| | PAK4; AKT1; PIK3R1; TNK2; MAP2K1; |
| | PAK3; ITGB3; CDC42; RND3; ITGA2; CRKL; |
| | BRAF; GSK3B; AKT3 |
| Acute Phase | IRAK1; SOD2; MYD88; TRAF6; ELK1; |
| Response | |
| Signaling | MAPK1; PTPN11; AKT2; IKBKB; P1K3CA; |
| | FOS; NFKB2; MAP3K14; PIK3CB; MAPK8; |
| | RIPK1; MAPK3; IL6ST; KRAS; MAPK13; |
| | IL6R; RELA; SOCS1; MAPK9; FTL; NR3C1; |
| | TRAF2; SERPINE1; MAPK14; TNF; RAF1; |
| | PDK1; IKBKG; RELB; MAP3K7; MAP2K2; |
| | AKT1; JAK2; PIK3R1; CHUK; STAT3; |
| | MAP2K1; NFKB1; FRAP1; CEBPB; JUN; |
| | AKT3; IL1R1; IL6 |
| PTEN Signaling | ITGAM; ITGA5; RAC1; PTEN; PRKCZ; |
| | BCL2L11; MAPK1; RAC2; AKT2; EGFR; |
| | IKBKB; CBL; P1K3CA; CDKN1B; PTK2; |
| | NFKB2; BCL2; PIK3CB; BCL2L1; MAPK3; |
| | ITGA1; KRAS; ITGB7; ILK; PDGFRB; INSR; |
| | RAF1; IKBKG; CASP9; CDKN1A; ITGB1; |
| | MAP2K2; AKT1; PIK3R1; CHUK; PDGFRA; |
| | PDPK1; MAP2K1; NFKB1; ITGB3; CDC42; |
| | CCND1; GSK3A; ITGA2; GSK3B; AKT3; |
| | FOXO1; CASP3; RPS6KB1 |
| p53 Signaling | PTEN; EP300; BBC3; PCAF; FASN; BRCA1; |
| | GADD45A; BIRC5; AKT2; P1K3CA; CHEK1; |
| | TP53INP1; BCL2; PIK3CB; PIK3C3; MAPK8; |
| | THBS1; ATR; BCL2L1; E2F1; PMAIP1; |
| | CHEK2; TNFRSF10B; TP73; RB1; HDAC9; |
| | CDK2; PIK3C2A; MAPK14; TP53; LRDD; |
| | CDKN1A; HIPK2; AKT1; RIK3R1; RRM2B; |
| | APAF1; CTNNB1; SIRT1; CCND1; PRKDC; |
| | ATM; SFN; CDKN2A; JUN; SNAI2; GSK3B; |
| | BAX; AKT3 |
| Aryl Hydrocarbon | HSPB1; EP300; FASN; TGM2; RXRA; |
| Receptor | MAPK1; NQO1; NCOR2; SP1; ARNT; |
| Signaling | CDKN1B; FOS; CHEK1; SMARCA4; NFKB2; |
| | MAPK8; ALDH1A1; ATR; E2F1; MAPK3; |
| | NRIP1; CHEK2; RELA; TP73; GSTP1; RB1; |
| | SRC; CDK2; AHR; NFE2L2; NCOA3; TP53; |
| | TNF; CDKN1A; NCOA2; APAF1; NFKB1; |
| | CCND1; ATM; ESR1; CDKN2A; MYC; JUN; |
| | ESR2; BAX; IL6; CYP1B1; HSP90AA1 |
| Xenobiotic | PRKCE; EP300; PRKCZ; RXRA; MAPK1; |
| Metabolism | |
| Signaling | NQO1; NCOR2; P1K3CA; ARNT; PRKCI; |
| | NFKB2; CAMK2A; PIK3CB; PPP2R1A; |
| | PIK3C3; MAPK8; PRKD1; ALDH1A1; |
| | MAPK3; NRIP1; KRAS; MAPK13; PRKCD; |
| | GSTP1; MAPK9; NOS2A; ABCB1; AHR; |
| | PPP2CA; FTL; NFE2L2; PIK3C2A; |
| | PPARGC1A; MAPK14; TNF; RAF1; CREBBP; |
| | MAP2K2; PIK3R1; PPP2R5C; MAP2K1; |
| | NFKB1; KEAP1; PRKCA; EIF2AK3; IL6; |
| | CYP1B1; HSP90AA1 |
| SAPK/JNK | PRKCE; IRAK1; PRKAA2; EIF2AK2; RAC1; |
| Signaling | |
| | ELK1; GRK6; MAPK1; GADD45A; RAC2; |
| | PLK1; AKT2; P1K3CA; FADD; CDK8; |
| | PIK3CB; PIK3C3; MAPK8; RIPK1; GNB2L1; |
| | IRS1; MAPK3; MAPK10; DAXX; KRAS; |
| | PRKCD; PRKAA1; MAPK9; CDK2; PIM1; |
| | PIK3C2A; TRAF2; TP53; LCK; MAP3K7; |
| | DYRK1A; MAP2K2; PIK3R1; MAP2K1; |
| | PAK3; CDC42; JUN; TTK; CSNK1A1; CRKL; |
| | BRAF; SGK |
| PPAr/RXR | PRKAA2; EP300; INS; SMAD2; TRAF6; |
| Signaling | |
| | PPARA; FASN; RXRA; MAPK1; SMAD3; |
| | GNAS; IKBKB; NCOR2; ABCA1; GNAQ; |
| | NFKB2; MAP3K14; STAT5B; MAPK8; IRS1; |
| | MAPK3; KRAS; RELA; PRKAA1; |
| | PPARGC1A; NCOA3; MAPK14; INSR; RAF1; |
| | IKBKG; RELB; MAP3K7; CREBBP; MAP2K2; |
| | JAK2; CHUK; MAP2K1; NFKB1; TGFBR1; |
| | SMAD4; JUN; IL1R1; PRKCA; IL6; |
| | HSP90AA1; ADIPOQ |
| NF-KB Signaling | IRAK1; EIF2AK2; EP300; INS; MYD88; |
| | PRKCZ: TRAF6; TBK1; AKT2; EGFR; IKBKB; |
| | P1K3CA; BTRC; NFKB2; MAP3K14; PIK3CB; |
| | PIK3C3; MAPK8; RIPK1; HDAC2; KRAS; |
| | RELA; PIK3C2A; TRAF2; TLR4: PDGFRB; |
| | TNF; INSR; LCK; IKBKG; RELB; MAP3K7; |
| | CREBBP; AKT1; PIK3R1; CHUK; PDGFRA; |
| | NFKB1; TLR2; BCL10; GSK3B; AKT3; |
| | TNFAIP3; IL1R1 |
| Neuregulin | ERBB4; PRKCE; ITGAM; ITGA5: PTEN; |
| Signaling | |
| | PRKCZ; ELK1; MAPK1; PTPN11; AKT2; |
| | EGFR; ERBB2; PRKCI; CDKN1B; STAT5B; |
| | PRKD1; MAPK3; ITGA1; KRAS; PRKCD; |
| | STAT5A; SRC; ITGB7; RAF1; ITGB1; |
| | MAP2K2; ADAM17; AKT1; PIK3R1; PDPK1; |
| | MAP2K1; ITGB3; EREG; FRAP1; PSEN1; |
| | ITGA2; MYC; NRG1; CRKL; AKT3; PRKCA; |
| | HSP90AA1; RPS6KB1 |
| Wnt & Beta catenin | CD44; EP300; LRP6; DVL3; CSNK1E; GJA1; |
| Signaling | SMO; AKT2; PIN1; CDH1; BTRC; GNAQ; |
| | MARK2; PPP2R1A; WNT11; SRC; DKK1; |
| | PPP2CA; SOX6; SFRP2: ILK; LEF1; SOX9; |
| | TP53; MAP3K7; CREBBP; TCF7L2; AKT1; |
| | PPP2R5C; WNT5A; LRP5; CTNNB1; TGFBR1; |
| | CCND1; GSK3A; DVL1; APC; CDKN2A; |
| | MYC; CSNK1A1; GSK3B; AKT3; SOX2 |
| Insulin Receptor | PTEN; INS; EIF4E; PTPN1; PRKCZ; MAPK1; |
| Signaling | TSC1; PTPN11; AKT2; CBL; P1K3CA; PRKCI; |
| | PIK3CB; PIK3C3; MAPK8; IRS1; MAPK3; |
| | TSC2; KRAS; EIF4EBP1; SLC2A4; PIK3C2A; |
| | PPP1CC; INSR; RAF1; FYN; MAP2K2; JAK1; |
| | AKT1; JAK2; PIK3R1; PDPK1; MAP2K1; |
| | GSK3A; FRAP1; CRKL; GSK3B; AKT3; |
| | FOXO1; SGK; RPS6KB1 |
| IL-6 Signaling | HSPB1; TRAF6; MAPKAPK2; ELK1; MAPK1; |
| | PTPN11; IKBKB; FOS; NFKB2: MAP3K14; |
| | MAPK8; MAPK3; MAPK10; IL6ST; KRAS; |
| | MAPK13; IL6R; RELA; SOCS1; MAPK9; |
| | ABCB1; TRAF2; MAPK14; TNF; RAF1; |
| | IKBKG; RELB; MAP3K7; MAP2K2; IL8; |
| | JAK2; CHUK; STAT3; MAP2K1; NFKB1; |
| | CEBPB; JUN; IL1R1; SRF; IL6 |
| Hepatic Cholestasis | PRKCE; IRAK1; INS; MYD88; PRKCZ; |
| | TRAF6; PPARA; RXRA; IKBKB; PRKCI; |
| | NFKB2; MAP3K14; MAPK8; PRKD1; |
| | MAPK10; RELA; PRKCD; MAPK9; ABCB1; |
| | TRAF2; TLR4; TNF; INSR; IKBKG; RELB; |
| | MAP3K7; IL8; CHUK; NR1H2; TJP2; NFKB1; |
| | ESR1; SREBF1; FGFR4; JUN; IL1R1; PRKCA; |
| | IL6 |
| IGF-1 Signaling | IGF1; PRKCZ; ELK1; MAPK1; PTPN11; |
| | NEDD4; AKT2; P1K3CA; PRKC1; PTK2; FOS; |
| | PIK3CB; PIK3C3; MAPK8; 1GF1R; IRS1; |
| | MAPK3; IGFBP7; KRAS; PIK3C2A; YWHAZ; |
| | PXN; RAF1; CASP9; MAP2K2; AKT1; |
| | PIK3R1; PDPK1; MAP2K1; IGFBP2; SFN; |
| | JUN; CYR61; AKT3; FOXO1; SRF; CTGF; |
| | RPS6KB1 |
| NRF2-mediated | PRKCE; EP300; SOD2; PRKCZ; MAPK1; |
| Oxidative | SQSTM1; NQO1; P1K3CA; PRKC1; FOS; |
| Stress Response | PIK3CB; P1K3C3; MAPK8; PRKD1; MAPK3; |
| | KRAS; PRKCD; GSTP1; MAPK9; FTL; |
| | NFE2L2; PIK3C2A; MAPK14; RAF1; |
| | MAP3K7; CREBBP; MAP2K2; AKT1; PIK3R1; |
| | MAP2K1; PPIB; JUN; KEAP1; GSK3B; ATF4; |
| | PRKCA; EIF2AK3; HSP90AA1 |
| Hepatic, Fibrosis/ | EDN1; IGF1; KDR; FLT1; SMAD2; FGFR1; |
| Hepatic | |
| Stellate Cell | MET; PGF; SMAD3; EGFR; FAS; CSF1; |
| Activation | |
| | NFKB2; BCL2; MYH9; IGF1R; IL6R; RELA; |
| | TLR4; PDGFRB; TNF; RELB; IL8; PDGFRA; |
| | NFKB1; TGFBR1; SMAD4; VEGFA; BAX; |
| | IL1R1; CCL2; HGF; MMP1; STAT1; IL6; |
| | CTGF; MMP9 |
| PPAR Signaling | EP300; INS; TRAF6; PPARA; RXRA; MAPK1; |
| | IKBKB; NCOR2; FOS; NFKB2; MAP3K14; |
| | STAT5B; MAPK3; NRIP1; KRAS; PPARG; |
| | RELA; STAT5A; TRAF2; PPARGC1A; |
| | PDGFRB; TNF; INSR; RAF1; IKBKG; RELB; |
| | MAP3K7; CREBBP; MAP2K2; CHUK; |
| | PDGFRA; MAP2K1; NFKB1; JUN; IL1R1; |
| | HSP90AA1 |
| Fc Epsilon RI | PRKCE; RAC1; PRKCZ; LYN; MAPK1; RAC2; |
| Signaling | |
| | PTPN11; AKT2; P1K3CA; SYK; PRKCI; |
| | PIK3CB; PIK3C3; MAPK8; PRKD1; MAPK3; |
| | MAPK10; KRAS; MAPK13; PRKCD; MAPK9; |
| | PIK3C2A; BTK; MAPK14; TNF; RAF1; FYN; |
| | MAP2K2; AKT1; PIK3R1; PDPK1; MAP2K1; |
| | AKT3; VAV3; PRKCA |
| G-Protein Coupled | PRKCE; RAP1A; RGS16; MAPK1; GNAS; |
| Receptor Signaling | AKT2; IKBKB; P1K3CA; CREB1; GNAQ; |
| | NFKB2; CAMK2A; PIK3CB; PIK3C3; MAPK3; |
| | KRAS; RELA; SRC; PIK3C2A; RAF1; IKBKG; |
| | RELB; FYN; MAP2K2; AKT1; PIK3R1; |
| | CHUK; PDPK1; STAT3; MAP2K1; NFKB1; |
| | BRAF; ATF4; AKT3; PRKCA |
| Inositol Phosphate | PRKCE; IRAK1; PRKAA2; EIF2AK2; PTEN; |
| Metabolism | GRK6; MAPK1; PLK1; AKT2; P1K3CA; CDK8; |
| | PIK3CB; PIK3C3; MAPK8; MAPK3; PRKCD; |
| | PRKAA1; MAPK9; CDK2; PIM1; PIK3C2A; |
| | DYRK1A; MAP2K2; PIP5K1A; PIK3R1; |
| | MAP2K1; PAK3; ATM; TTK; CSNK1A1; |
| | BRAF; SGK |
| PDGF Signaling | EIF2AK2; ELK1; ABL2; MAPK1; P1K3CA; |
| | FOS; PIK3CB; PIK3C3; MAPK8; CAV1; ABL1; |
| | MAPK3; KRAS; SRC; PIK3C2A; PDGFRB; |
| | RAF1; MAP2K2; JAK1; JAK2; PIK3R1; |
| | PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; |
| | MAPK1; PGF; AKT2; P1K3CA; ARNT; PTK2; |
| | BCL2; PIK3CB; PIK3C3; BCL2L1; MAPK3; |
| | KRAS; HIF1A; NOS3; PIK3C2A; PXN; RAF1; |
| | MAP2K2; ELAVL1; AKT1; PIK3R1; MAP2K1; |
| | SFN; VEGFA; AKT3; FOXO1; PRKCA |
| Natural Killer Cell | PRKCE; RAC1; PRKCZ; MAPK1; RAC2; |
| Signaling | PTPN11; KIR2DL3; AKT2; P1K3CA; SYK; |
| | PRKCI; PIK3CB; PIK3C3; PRKD1; MAPK3; |
| | KRAS; PRKCD; PTPN6; PIK3C2A; LCK; |
| | RAF1; FYN; MAP2K2; PAK4; AKT1; PIK3R1; |
| | MAP2K1; PAK3; AKT3; VAV3; PRKCA |
| Cell Cycle: G1/S | HDAC4; SMAD3; SUV39H1; HDAC5; |
| Checkpoint | CDKN1B; BTRC; ATR; ABL1; E2F1; HDAC2; |
| Regulation | |
| | HDAC7A; RB1; HDAC11; HDAC9; CDK2; |
| | E2F2; HDAC3; TP53; CDKN1A; CCND1; |
| | E2F4; ATM; RBL2; SMAD4; CDKN2A; MYC; |
| | NRG1; GSK3B; RBL1; HDAC6 |
| T Cell Receptor | RAC1; ELK1; MAPK1; IKBKB; CBL; P1K3CA; |
| Signaling | FOS; NFKB2; PIK3CB; PIK3C3; MAPK8; |
| | MAPK3; KRAS; RELA, PIK3C2A; BTK; LCK; |
| | RAF1; IKBKG; RELB, FYN; MAP2K2; |
| | PIK3R1; CHUK; MAP2K1; NFKB1; ITK; |
| | BCL10; JUN; VAV3 |
| Death Receptor | CRADD; HSPB1; BID; BIRC4; TBK1; IKBKB; |
| Signaling | |
| | FADD; FAS; NFKB2; BCL2; MAP3K14; |
| | MAPK8; RIPK1; CASP8; DAXX; TNFRSF10B; |
| | RELA; TRAF2; TNF; IKBKG; RELB; CASP9; |
| | CHUK; APAF1; NFKB1; CASP2; BIRC2; |
| | CASP3; BIRC3 |
| FGF Signaling | RAC1; FGFR1; MET; MAPKAPK2; MAPK1; |
| | PTPN11; AKT2; P1K3CA; CREB1; PIK3CB; |
| | PIK3C3; MAPK8; MAPK3; MAPK13; PTPN6; |
| | PIK3C2A; MAPK14; RAF1; AKT1; PIK3R1; |
| | STAT3; MAP2K1; FGFR4; CRKL; ATF4; |
| | AKT3; PRKCA; HGF |
| GM-CSF Signaling | LYN; ELK1; MAPK1; PTPN11; AKT2; |
| | P1K3CA; CAMK2A; STAT5B; PIK3CB; |
| | PIK3C3; GNB2L1; BCL2L1; MAPK3; ETS1; |
| | KRAS; RUNX1; PIM1; PIK3C2A; RAF1; |
| | MAP2K2; AKT1; JAK2; PIK3R1; STAT3; |
| | MAP2K1; CCND1; AKT3; STAT1 |
| Amyotrophic Lateral | BID; IGF1; RAC1; BIRC4; PGF; CAPNS1; |
| Sclerosis Signaling | CAPN2; P1K3CA; BCL2; PIK3CB; PIK3C3; |
| | BCL2L1; CAPN1; PIK3C2A; TP53; CASP9; |
| | PIK3R1; RAB5A; CASP1; APAF1; VEGFA; |
| | BIRC2; BAX; AKT3; CASP3; BIRC3 |
| JAK/Stat Signaling | PTPN1; MAPK1; PTPN11; AKT2; P1K3CA; |
| | STAT5B; PIK3CB; PIK3C3; MAPK3; KRAS; |
| | SOCS1; STAT5A; PTPN6; PIK3C2A; RAF1; |
| | CDKN1A; MAP2K2; JAK1; AKT1; JAK2; |
| | PIK3R1; STAT3; MAP2K1; FRAP1; AKT3; |
| | STAT1 |
| Nicotinate and | PRKCE; IRAK1; PRKAA2; EIF2AK2; GRK6; |
| Nicotinamide | MAPK1; PLK1; AKT2; CDK8; MAPK8; |
| Metabolism | MAPK3; PRKCD; PRKAA1; PBEF1; MAPK9; |
| | CDK2; PIM1; DYRK1A; MAP2K2; MAP2K1; |
| | PAK3; NT5E; TTK; CSNK1A1; BRAF; SGK |
| Chemokine | CXCR4; ROCK2; MAPK1; PTK2; FOS; CFL1; |
| Signaling | |
| | GNAQ; CAMK2A; CXCL12; MAPK8; MAPK3; |
| | KRAS; MAPK13; RHOA; CCR3; SRC; |
| | PPP1CC; MAPK14; NOX1; RAF1; MAP2K2; |
| | MAP2K1; JUN; CCL2; PRKCA |
| IL-2 Signaling | ELK1; MAPK1; PTPN11; AKT2; P1K3CA; |
| | SYK; FOS; STAT5B; PIK3CB; PIK3C3; |
| | MAPK8; MAPK3; KRAS; SOCS1; STAT5A; |
| | PIK3C2A: LCK; RAF1; MAP2K2; JAK1; |
| | AKT1; PIK3R1; MAP2K1; JUN; AKT3 |
| Synaptic Long | PRKCE; IGF1; PRKCZ; PRDX6; LYN; |
| Term | |
| Depression | MAPK1; GNAS; PRKC1; GNAQ; PPP2R1A; |
| | IGF1R; PRKID1; MAPK3; KRAS; GRN; |
| | PRKCD; NOS3; NOS2A; PPP2CA; YWHAZ; |
| | RAF1; MAP2K2; PPP2R5C; MAP2K1; PRKCA |
| Estrogen Receptor | TAF4B; EP300; CARM1; PCAF; MAPK1; |
| Signaling | NCOR2; SMARCA4; MAPK3; NRIP1; KRAS; |
| | SRC; NR3C1; HDAC3; PPARGC1A; RBM9; |
| | NCOA3; RAF1; CREBBP; MAP2K2; NCOA2; |
| | MAP2K1; PRKDC; ESR1; ESR2 |
| Protein | TRAF6; SMURF1; BIRC4; BRCA1; UCHL1; |
| Ubiquitination | |
| Pathway | NEDD4; CBL; UBE2I; BTRC; HSPA5; USP7; |
| | USP10; FBXW7; USP9X; STUB1; USP22; |
| | B2M; BIRC2; PARK2; USP8; USP1; VHL; |
| | HSP90AA1; BIRC3 |
| IL-10 Signaling | TRAF6; CCR1; ELK1; IKBKB; SP1; FOS; |
| | NFKB2; MAP3K14; MAPK8; MAPK13; RELA; |
| | MAPK14; TNF; IKBKG; RELB; MAP3K7; |
| | JAK1; CHUK; STAT3; NFKB1; JUN; IL1R1; |
| | IL6 |
| VDR/RXR | PRKCE; EP300; PRKCZ; RXRA; GADD45A; |
| Activation | |
| | HES1; NCOR2; SP1; PRKC1; CDKN1B; |
| | PRKD1; PRKCD; RUNX2; KLF4; YY1; |
| | NCOA3; CDKN1A; NCOA2; SPP1; LRP5; |
| | CEBPB; FOXO1; PRKCA |
| TGF-beta Signaling | EP300; SMAD2; SMURF1; MAPK1; SMAD3; |
| | SMAD1; FOS; MAPK8; MAPK3; KRAS; |
| | MAPK9; RUNX2; SERPINE1; RAF1; |
| | MAP3K7; CREBBP; MAP2K2; MAP2K1; |
| | TGFBR1; SMAD4; JUN; SMAD5 |
| Toll-like Receptor | IRAK1; EIF2AK2; MYD88; TRAF6; PPARA; |
| Signaling | ELK1; IKBKB; FOS; NFKB2; MAP3K14; |
| | MAPK8; MAPK13; RELA; TLR4; MAPK14; |
| | IKBKG; RELB; MAP3K7; CHUK; NFKB1; |
| | TLR2; JUN |
| p38 MAPK | HSPB1; IRAK1; TRAF6; MAPKAPK2; ELK1; |
| Signaling | |
| | FADD; FAS; CREB1; DDIT3; RPS6KA4; |
| | DAXX; MAPK13; TRAF2; MAPK14; TNF; |
| | MAP3K7; TGFBR1; MYC; ATF4; IL1R1; SRF; |
| | STAT1 |
| Neurotrophin/TRK | NTRK2; MAPK1; PTPN11; P1K3CA; CREB1; |
| Signaling | FOS; PIK3CB; PIK3C3; MAPK8; MAPK3; |
| | KRAS; PIK3C2A; RAF1; MAP2K2; AKT1; |
| | PIK3R1; PDPK1; MAP2K1; CDC42; JUN; |
| | ATF4 |
| FXR/RXR | INS; PPARA; FASN; RXRA; AKT2; SDC1; |
| Activation | |
| | MAPK8; APOB; MAPK10; PPARG; MTTP; |
| | MAPK9; PPARGC1A; TNF; CREBBP; AKT1; |
| | SREBF1; FGFR4; AKT3; FOXO1 |
| Synaptic Long | PRKCE; RAP1A; EP300; PRKCZ; MAPK1; |
| Term | |
| Potentiation | CREB1; PRKC1; GNAQ; CAMK2A; PRKD1; |
| | MAPK3; KRAS; PRKCD; PPP1CC; RAF1; |
| | CREBBP; MAP2K2; MAP2K1; ATF4; PRKCA |
| Calcium Signaling | RAP1A; EP300; HDAC4; MAPK1; HDAC5; |
| | CREB1; CAMK2A; MYH9; MAPK3; HDAC2; |
| | HDAC7A; HDAC11; HDAC9; HDAC3; |
| | CREBBP; CALR; CAMKK2; ATF4; HDAC6 |
| EGF Signaling | ELK1; MAPK1; EGFR; P1K3CA; FOS; |
| | PIK3CB; PIK3C3; MAPK8; MAPK3; PIK3C2A; |
| | RAF1; JAK1; PIK3R1; STAT3; MAP2K1; JUN; |
| | PRKCA; SRF; STAT1 |
| Hypoxia Signaling | EDN1; PTEN; EP300; NQO1; UBE2I; CREB1; |
| in the | |
| Cardiovascular | ARNT; HIF1A; SLC2A4; NOS3; TP53; LDHA; |
| System | |
| | AKT1; ATM; VEGFA; JUN; ATF4; VHL; |
| | HSP90AA1 |
| LPS/IL-1 Mediated | IRAK1; MYD88; TRAF6; PPARA; RXRA; |
| Inhibition | ABCA1, MAPK8; ALDH1A1; GSTP1; MAPK9; |
| of RXR Function | ABCB1; TRAF2; TLR4; TNF; MAP3K7; |
| | NR1H2; SREBF1; JUN; IL1R1 |
| LXR/RXR | FASN; RXRA; NCOR2; ABCA1; NFKB2; |
| Activation | |
| | IRF3; RELA; NOS2A; TLR4; TNF; RELB; |
| | LDLR; NR1H2; NFKB1; SREBF1; IL1R1; |
| | CCL2; IL6; MMP9 |
| Amyloid Processing | PRKCE; CSNK1E; MAPK1; CAPNS1; AKT2; |
| | CAPN2; CAPN1; MAPK3; MAPK13; MAPT; |
| | MAPK14; AKT1; PSEN1; CSNK1A1; GSK3B; |
| | AKT3; APP |
| IL-4 Signaling | AKT2; P1K3CA; PIK3CB; PIK3C3; IRS1; |
| | KRAS; SOCS1; PTPN6; NR3C1; PIK3C2A; |
| | JAK1; AKT1; JAK2; PIK3R1; FRAP1; AKT3; |
| | RPS6KB1 |
| Cell Cycle: G2/M | EP300; PCAF; BRCA1; GADD45A; PLK1; |
| DNA | |
| Damage Checkpoint | BTRC; CHEK1; ATR; CHEK2; YWHAZ; TP53; |
| Regulation | CDKN1A; PRKDC; ATM; SFN; CDKN2A |
| Nitric Oxide | KDR; FLT1; PGF; AKT2; P1K3CA; PIK3CB; |
| Signaling in | |
| the | PIK3C3; CAV1; PRKCD; NOS3; PIK3C2A; |
| Cardiovascular | AKT1; PIK3R1; VEGFA; AKT3; HSP90AA1 |
| System | |
| Purine Metabolism | NME2; SMARCA4; MYH9; RRM2; ADAR; |
| | EIF2AK4; PKM2; ENTPD1; RAD51; RRM2B; |
| | TJP2; RAD51C; NT5E; POLD1; NME1 |
| CAMP-mediated | RAP1A; MAPK1; GNAS; CREB1; CAMK2A; |
| Signaling | MAPK3; SRC; RAF1; MAP2K2; STAT3; |
| | MAP2K1; BRAF; ATF4 |
| Mitochondrial | SOD2; MAPK8; CASP8; MAPK10; MAPK9; |
| Dysfunction | CASP9; PARK7; PSEN1; PARK2; APP; CASP3 |
| Notch Signaling | HES1; JAG1; NUMB; NOTCH4; ADAM17; |
| | NOTCH2; PSEN1; NOTCH3; NOTCH1; DLL4 |
| Endoplasmic | HSPA5; MAPK8; XBP1; TRAF2; ATF6; |
| Reticulum | |
| Stress Pathway | CASP9; ATF4; EIF2AK3; CASP3 |
| Pyrimidine | NME2; AICDA; RRM2; EIF2AK4; ENTPD1; |
| Metabolism | |
| | RRM2B; NT5E; POLD1; NME1 |
| Parkinson's | UCHL1; MAPK8; MAPK13; MAPK14; CASP9; |
| Signaling | |
| | PARK7; PARK2; CASP3 |
| Cardiac & Beta | GNAS; GNAQ; PPP2R1A; GNB2L1; PPP2CA; |
| Adrenergic | PPP1CC; PPP2R5C |
| Signaling | |
| Glycolysis/Gluco- | HK2; GCK; GPI; ALDH1A1; PKM2; LDHA; |
| neogenesis | HK1 |
| Interferon Signaling | IRF1; SOCS1; JAK1; JAK2; IFITM1; STAT1; |
| | IFIT3 |
| Sonic Hedgehog | ARRB2; SMO; GLI2; DYRK1A; GLI1; GSK3B; |
| Signaling | DYRKIB |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; |
| Degradation | SPHK2 |
| Tryptophan | SIAH2; PRMT5; NEDD4; ALDH1A1; CYP1B1; |
| Metabolism | |
| | SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine | DNMT1; DNMT3B; AHCY; DNMT3A |
| Metabolism | |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and | HK2; GCK; HK1 |
| Mannose | |
| Metabolism | |
| Galactose | HK2; GCK; HK1 |
| Metabolism | |
| Stilbene, Coumarine | PRDX6; PRDX1; TYR |
| and | |
| Lignin Biosynthesis | |
| Antigen Presentation | CALR; B2M |
| Pathway | |
| Biosynthesis of | NQO1; DHCR7 |
| Steroids | |
| Butanoate | ALDH1A1; NLGN1 |
| Metabolism | |
| Citrate Cycle | IDH2; IDH1 |
| Fatty Acid | ALDH1A1; CYP1B1 |
| Metabolism | |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics | |
| by Cytochrome p450 | |
| Methane Metabolism | PRDX6; PRDX1 |
| Phenylalanine | PRDX6; PRDX1 |
| Metabolism | |
| Propanoate | ALDH1A1; LDHA |
| Metabolism | |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and | PRMT5 |
| Estrogen | |
| Metabolism | |
| Ascorbate and | ALDH1A1 |
| Aldarate | |
| Metabolism | |
| Bile Acid | ALDH1A1 |
| Biosynthesis | |
| Cysteine Metabolism | LDHA |
| Fatty Acid | FASN |
| Biosynthesis | |
| Glutamate Receptor | GNB2L1 |
| Signaling | |
| NRF2-mediated | PRDX1 |
| Oxidative | |
| Stress Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin | TYR |
| Metabolism | |
| Tyrosine Metabolism | PRMT5, TYR |
| Ubiquinone | PRMT5 |
| Biosynthesis | |
| Valine, Leucine and | ALDH1A1 |
| Isoleucine | |
| Degradation | |
| Glycine, Serine and | CHKA |
| Threonine | |
| Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; |
| | Grk2; Trpa1; Pomc; Cgrp; Crf; Pka; Era; Nr2b; |
| | TRPM5; Prkaca; Prkacb; Prkar1a; Prkar2a |
| Mitochondrial | AIF; CytC; SMAC (Diablo); Aifm-1; Aifm-2 |
| Function | |
| Developmental | BMP-4; Chordin (Chrd); Noggin (Nog); WNT |
| Neurology | (Wnt2; Wnt2b; Wnt3a; Wnt4; Wnt5a; Wnt6; |
| | Wnt7b; Wnt8b; Wnt9a; Wnt9b; Wnt10a; |
| | Wnt10b; Wnt16); beta-catenin; Dkk-1; Frizzled |
| | related proteins; Otx-2; Gbx2; FGF-8; Reelin; |
| | Dab1; unc-86 (Pou4f1 or Brn3a); Numb; Reln |

Embodiments of the invention also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (New insights into repeat instability: role of RNA*DNA hybrids. McIvor E I, Polak U, Napierala M. RNA Biol. 2010 September-October; 7(5):551-8). The CRISPR-Cas system may be harnessed to correct these defects of genomic instability.

Several further aspects of the invention relate to correcting defects associated with a wide range of genetic diseases which are further described on the website of the National Institutes of Health under the topic subsection Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease. Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

In some embodiments, the condition may be neoplasia. In some embodiments, where the condition is neoplasia, the genes to be targeted are any of those listed in Table A (in this case PTEN asn so forth). In some embodiments, the condition may be Age-related Macular Degeneration. In some embodiments, the condition may be a Schizophrenic Disorder. In some embodiments, the condition may be a Trinucleotide Repeat Disorder. In some embodiments, the condition may be Fragile X Syndrome. In some embodiments, the condition may be a Secretase Related Disorder. In some embodiments, the condition may be a Prion-related disorder. In some embodiments, the condition may be ALS. In some embodiments, the condition may be a drug addiction. In some embodiments, the condition may be Autism. In some embodiments, the condition may be Alzheimer's Disease. In some embodiments, the condition may be inflammation. In some embodiments, the condition may be Parkinson's Disease.

Examples of proteins associated with Parkinson's disease include but are not limited to α-synuclein, DJ-1, LRRK2, PINK1, Parkin, UCHL1, Synphilin-1, and NURR1.

Examples of addiction-related proteins may include ABAT for example.

Examples of inflammation-related proteins may include the monocyte chemoattractant protein-1 (MCP1) encoded by the Ccr2 gene, the C-C chemokine receptor type 5 (CCR5) encoded by the Ccr5 gene, the IgG receptor IIB (FCGR2b, also termed CD32) encoded by the Fcgr2b gene, or the Fc epsilon R1g (FCER1g) protein encoded by the Fcer1g gene, for example.

Examples of cardiovascular diseases associated proteins may include IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), or CTSK (cathepsin K), for example.

Examples of Alzheimer's disease associated proteins may include the very low density lipoprotein receptor protein (VLDLR) encoded by the VLDLR gene, the ubiquitin-like modifier activating enzyme 1 (UBA1) encoded by the UBA1 gene, or the NEDD8-activating enzyme E1 catalytic subunit protein (UBE1C) encoded by the UBA3 gene, for example.

Examples of proteins associated Autism Spectrum Disorder may include the benzodiazapine receptor (peripheral) associated protein 1 (BZRAP1) encoded by the BZRAP1 gene, the AF4/FMR2 family member 2 protein (AFF2) encoded by the AFF2 gene (also termed MFR2), the fragile X mental retardation autosomal homolog 1 protein (FXR1) encoded by the FXR1 gene, or the fragile X mental retardation autosomal homolog 2 protein (FXR2) encoded by the FXR2 gene, for example.

Examples of proteins associated Macular Degeneration may include the ATP-binding cassette, sub-family A (ABC1) member 4 protein (ABCA4) encoded by the ABCR gene, the apolipoprotein E protein (APOE) encoded by the APOE gene, or the chemokine (C-C motif) Ligand 2 protein (CCL2) encoded by the CCL2 gene, for example.

Examples of proteins associated Schizophrenia may include NRG1, ErbB4, CPLX1, TPH1, TPH2, NRXN1, GSK3A, BDNF, DISC1, GSK3B, and combinations thereof.

Examples of proteins involved in tumor suppression may include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, or Notch 4, for example.

Examples of proteins associated with a secretase disorder may include PSENEN (presenilin enhancer 2 homolog (C. elegans)), CTSB (cathepsin B), PSEN1 (presenilin 1), APP (amyloid beta (A4) precursor protein), APH1B (anterior pharynx defective 1 homolog B (C. elegans)), PSEN2 (presenilin 2 (Alzheimer disease 4)), or BACE1 (beta-site APP-cleaving enzyme 1), for example.

Examples of proteins associated with Amyotrophic Lateral Sclerosis may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins associated with prion diseases may include SODI (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins related to neurodegenerative conditions in prion disorders may include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), or ADRA1D (Alpha-1D adrenergic receptor for Alpha-1D adrenoreceptor), for example.

Examples of proteins associated with Immunodeficiency may include A2M [alpha-2-macroglobulin]; AANAT [arylalkylamine N-acetyltransferase]; ABCA1 [ATP-binding cassette, sub-family A (ABC1), member 1]; ABCA2 [ATP-binding cassette, sub-family A (ABC1), member 2]; or ABCA3 [ATP-binding cassette, sub-family A (ABC1), member 3]; for example.

Examples of proteins associated with Trinucleotide Repeat Disorders include AR (androgen receptor), FMR1 (fragile X mental retardation 1), HTT (huntingtin), or DMPK (dystrophia myotonica-protein kinase), FXN (frataxin), ATXN2 (ataxin 2). for example.

Examples of proteins associated with Neurotransmission Disorders include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), or HTR2c (5-hydroxytryptamine (serotonin) receptor 2C), for example.

Examples of neurodevelopmental-associated sequences include A2BP1 [ataxin 2-binding protein 1], AADAT [aminoadipate aminotransferase], AANAT [arylalkylamine N-acetyltransferase], ABAT [4-aminobutyrate aminotransferase], ABCA1 [ATP-binding cassette, sub-family A (ABC1), member 1], or ABCA13 [ATP-binding cassette, sub-family A (ABC1), member 13], for example.

Further examples of preferred conditions treatable with the present system include may be selected from: Aicardi-Goutieres Syndrome; Alexander Disease; Allan-Hemdon-Dudley Syndrome; POLG-Related Disorders; Alpha-Mannosidosis (Type II and III); Alström Syndrome; Angelman; Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A 1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy, Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; and Xeroderma Pigmentosum.

As will be apparent, it is envisaged that the present system can be used to target any polynucleotide sequence of interest. Some examples of conditions or diseases that might be usefully treated using the present system are included in the Tables above and examples of genes currently associated with those conditions are also provided there. However, the genes exemplified are not exhaustive.

While the present invention has been described above in relation to CRISPR-Cas9 systems, the present invention also contemplates the use of other systems for introducing double stranded breaks into a target sequence is host cell genome followed by insertion of a sequence of interest by homologous recombination. As above, these systems include co-expression of an exogenous recombinase to increase the efficiency of homologous recombination.

In some embodiments, targeted zinc finger nucleases (ZFNs) are utilized to introduce double stranded breaks as a site for homologous recombination. See, e.g., Carroll et al., Genetics (2011) 188:773-782; Meyer et al., Proc. Nat'l. Acad. Sci. (2010) 107(34):15022-15026; Porteus M H, Carroll D (2005) Gene targeting using zinc finger nucleases. Nat Biotechnol 23:967-973; Geurts A M, et al. (2009) Knockout rats via embryo microinjection of zinc-finger nucleases. Science 325:433; Mashimo T, et al. (2010) Generation of knockout rats with X-linked severe combined immunodeficiency (X-SCID) using zinc-finger nucleases. PLoS One 5:e8870; Meng X, Noyes M B, Zhu L J, Lawson N D, Wolfe S A (2008) Targeted gene inactivation in zebrafish using engineered zinc-finger nucleases. Nat Biotechnol 26:695-701; Rouet P, Smith F, Jasin M (1994) Expression of a site-specific endonuclease stimulates homologous recombination in mammalian cells. Proc Natl Acad Sci USA 91:6064-6068; Hockemeyer D, et al. (2009) Efficient targeting of expressed and silent genes in human ESCs and iPSCs using zinc-finger nucleases. Nat Biotechnol 27:851-857; Porteus MH, Baltimore D (2003) Chimeric nucleases stimulate gene targeting in human cells. Science 300:763; Santiago Y, et al. (2008) Targeted gene knockout in mammalian cells by using engineered zinc-finger nucleases. Proc Natl Acad Sci USA 105:5809-5814; Umov F D, et al. (2005) Highly efficient endogenous human gene correction using designed zinc-finger nucleases. Nature 435:646-651; each of which is incorporated herein by reference in its entirety. Zinc-finger nucleases (ZFN) link a DNA binding domain of the zinc-finger type to the nuclease domain of Fok I and enable the induction of double-strand breaks (DSBs) at preselected genomic sites. DSBs closed by the error-prone, nonhomologous end-joining (NHEJ) DNA repair pathway frequently exhibit nucleotide deletions and insertions at the cleavage site. The present invention addresses this problem by co-expression of an exogenous recombinase.

In some embodiments, targeted transcription activator-like effector (TALE) nucleases are utilized to introduce double stranded breaks as a site for homologous recombination. See, e.g., Shin et al., Development (2014) 141:3807-3818; Boch et al. (2009) Science 326, 1509-1512; and Moscou and Bogdanove (2009) Science 326, 1501; each of which is incorporated by reference herein in its entirety. In still other embodiments, targeted meganucleases are utilized. See, e.g., Mol Cell Biol. 1994 December; 14(12):8096-106. Introduction of double-strand breaks into the genome of mouse cells by expression of a rare-cutting endonuclease. Rouet P1, Smih F, Jasin M.

In some embodiments, the present invention provides a cell comprising the system described above. In some embodiments, the present invention provides for use of the system to treat a disease by altering expression of gene in a target cell or editing the genome of a target cell.

### EXAMPLES

### Example 1

### Methods

### Cell Culture & Reporter Cell Lines

### Cell Culture

HEK293T, RPE1 and BJ-5ta cells were cultured at 37° C. in a humidified incubator in DMEM (Thermo Fisher Scientific) supplemented with 10% fetal bovine serum (FBS; Thermo Fisher Scientific) and 1% Penicillin-Streptomycin (Thermo Fisher Scientific). After electroporation, cells were cultured in DMEM (Thermo Fisher Scientific) supplemented with 10% FBS (Thermo Fisher Scientific) but without penicillin-streptomycin. Cells were split routinely twice a week using TrypLE™ Express Enzyme (Thermo Fisher Scientific).

### RPE-1 and HEK293T GFP Reporter Cell Lines

For easy detection of CRISPR-Cas9-mediated homologous recombination, we created a cassette encoding a) Zeocin resistance gene for selecting stable integrants, b) mutant Green Fluorescent Protein (GFP) (37A-38A-39A) sequence, c) Blue Fluorescent protein (BFP) or Red Fluorescent protein (RFP) sequence separated from GFP by a 2A self-cleaving peptide and c) sgRNA targeting the mutant GFP. When Cas9 along with a repair template correcting the GFP mutation is introduced into these cell lines, gene correction will give rise to functional GFP. GFP fluorescence was measured by FACS after 5 days if Cas9 is delivered as a plasmid, or 4 days if Cas9 is delivered as ribonucleoprotein.

The reporter cassette sequence was cloned to pLenti sgRNA(MS2) plasmid backbone (Addgene #61427). The cassette sequence is shown in Supplementary Appendix III and the plasmid will be deposited to Addgene.

### Lentivirus Production and Transduction

The reporter cassette was packaged into lentiviral particles by transfecting HEK 293T cells with the reporter cassette plasmid and the two packaging plasmids psPAX2 (Addgene #12260) and pCMV-VSV-G (Addgene #8454) in equimolar ratios. After 48 hours, the virus-containing supernatant was concentrated 40-fold using Lenti-X concentrator (Clontech). Single use aliquots were prepared and stored at −140° C.

The cassette was transduced to HEK293T and RPE1 cells by lentivirus at low multiplicity of infection (MOI). The cells were propagated in Zeocin (500 ug/ml) selection media for two weeks. After selection, single-cell clones were obtained by sorting BFP+ cells to 96-well plates by flow cytometry. The clones were expanded and frozen for future use.

BJ-5ta and HEK293T sgRNA cell lines

The cell lines express a guide targeting one of the four endogenous loci: RNF2, ELANE, Enh1-4 and CTCF1. The CRISPR RNA sequences (shown in Supplementary Table 3) were synthesized by Eurofins Genomics and cloned to pLentiPuro plasmid (Addgene #52963) according to published instructions¹. The plasmids' identities were verified by Sanger sequencing.

The lentiviruses were prepared and transduced to HEK293T and BJ-5ta cells as described in 1.4. The cells were selected with 10 μg/ml puromycin (Thermo Fisher Scientific) or 10 μg/ml blasticidin (Sigma Aldrich) for 7 days prior to aliquoting and freezing.

Arrayed DNA repair protein CRISPR screens

### DNA Repair Protein Library

The full-length open reading frames (ORFs) were either picked from the human orfeome (hORF8.1) or synthesized by Genscript Inc. Since Cas9 is a large protein of >2000 amino acids (AAs), DNA repair proteins that exceeded the size of 600 AA were processed into smaller fragments (100-500AA) in between the domain boundaries. The fragments were synthesized by Genscript Inc.

### Expression Plasmids

By utilizing the commercial pcDNA-DEST40 backbone (Invitrogen), we constructed a custom mammalian expression plasmid containing a WTCas9 sequence with N- and C-terminal nuclear localization signals, followed by a Gateway recombination site (referred as Cas9-GW plasmid). In addition, we created a pcDNA-DEST40 plasmid that contains a MS2 sequence downstream of the Gateway recombination site (referred as CT-MS2 plasmid). The Cas9 and MS2 sequences are in Supplementary Appendix I and Supplementary Appendix II. Genscript Inc. performed the insert synthesis and cloning to pcDNA-DEST40 backbone.

### High-Throughput Gateway Cloning and Plasmid Preparation

The DNA repair protein inserts were cloned to Cas9-GW and CT-MS2 plasmids in 96-well plates in 5 ul reactions, with 1 ul Gateway LR clonase II enzyme mix (Invitrogen), 50 ng entry plasmid and 100 ng destination plasmids, respectively, and Tris-EDTA (TE) buffer (pH 8) added to a final volume of 5 ul. After overnight incubation, we transformed 1 ul of the reaction mix to 15-20 ul of DH5-α competent cells (Invitrogen). Plasmids were extracted in 96-well format using the Wizard SV 96 plasmid miniprep kit (Promega) according to manufacturer's instructions using the Biomek FXP liquid handling system (Beckman Coulter), and eluted to 100 ul TE buffer (pH 8). This yielded a destination plasmid concentration of ˜150 ng in the majority of the wells.

### Cell Transfections

HEK293T cells containing the GFP-BFP-sgRNA reporter cassette were split into 24-well plates at ˜100 000 cells/well, 500 ul final volume. The next day, we replaced the media and prepared the transfection complexes in 96-well plates using Fugene HD (Promega) with the following modifications: with 25 ul Opti-MEM (Thermo Fisher), 2 ul Fugene HD (Promega), 400 ng plasmid and 12.5 pmol repair DNA (synthesized by Eurofins Genomics). After 20 min incubation, the cells were transfected in triplicate, with the end volume of 25 ul transfection mix in each well. Transfections were conducted with Biomek FXP liquid handling system (Beckman Coulter). The cells were grown for 5 days and GFP expression evaluated by FACS as described in 3.

For co-expressing Cas9WT with MS2-tagged DNA repair proteins, the cells were transfected as described above, with the following modifications: 25 ul Opti-MEM (Thermo Fisher), 2 ul Fugene HD (Promega), 180 ng Cas9 WT plasmid², 200 ng MS2 plasmid, and 12.5 pmol repair DNA.

### Fluorescence-Activated Cell Sorting (FACS)

For the detection of corrected GFP (see 1.2), the Cas9 transfected cells were cultured for days, trypsinized, resuspended in warm culture medium and immediately subjected to FACS. The data was acquired by CyAn II flow cytometer (Beckman Coulter) coupled with Hypercyte robotics (Intellicyt) or iQue screener plus (Intellicyt).

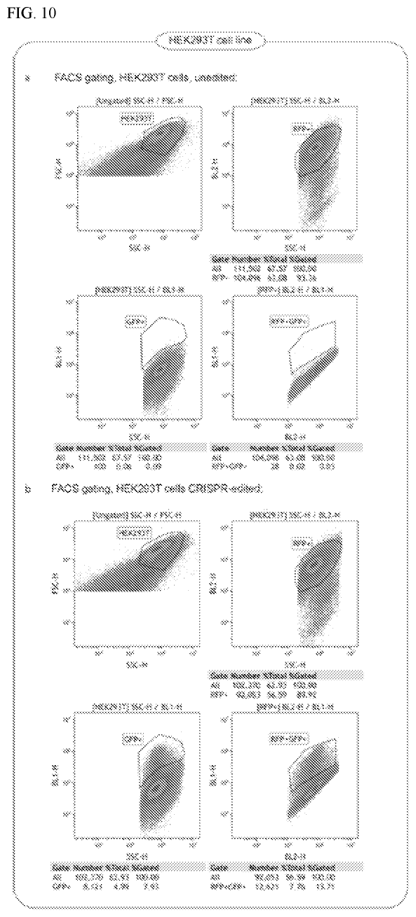

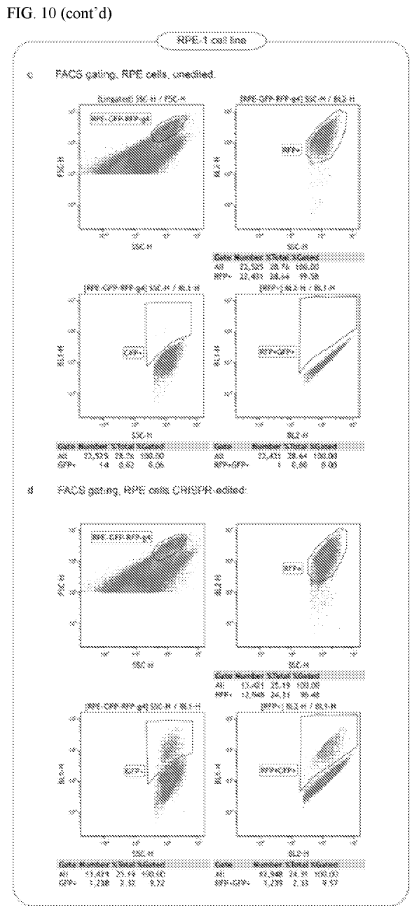

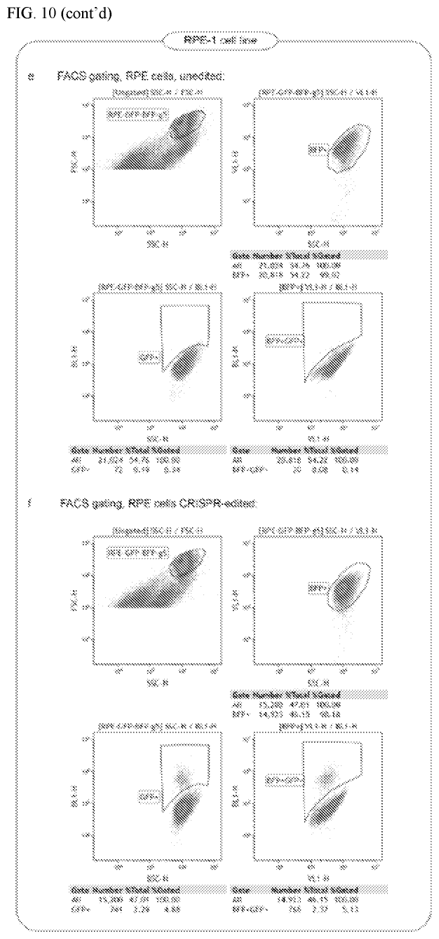

The well information was extracted from the plate fcs file using custom R code or Kaluza v.1.2 or 2.1 (Beckman Coulter). The number of GFP+ cells per well was obtained by batch gating. The gates used in analyzing the screen FACS data are shown on FIG. 10. The effect size for each Cas9 fusion was calculated by normalizing the GFP expression of the individual well either to the average GFP expression of all wells in the plate or to the overall experimental average. The statistical significance was calculated using standard one-way Anova test (Table 4).

### Screen Validations

### Cas9WT Fusion Protein Validation in HEK293T and RPE1 Reporter Cells

We chose 60 Cas9WT-DNA repair fusion constructs for validation. We picked the constructs from original plates using Biomek i7 robotics and prepared new minipreps from the original constructs using Wizard SV 96 Plasmid DNA Purification System (Promega), according to manufacturer's instructions. The constructs were Sanger sequenced to validate construct identity.

HEK293T cells containing the GFP-RFP-sgRNA reporter cassette were transfected as described in 2.4, with the following modifications: seeding on the 48 well plate, 30 000 cells/well; transfection mix: 12.5 ul Opti-MEM (Thermo Fisher), 1.25 ul Fugene HD (Promega), 250 ng plasmid and 6 pmol repair DNA (synthesized by Eurofins Genomics). After 20 min incubation, the cells were transfected in four replicas. The transfections were performed in parallel plates to account for batch variation between cell culture plates. Transfections were conducted with Biomek i7 liquid handling system (Beckman Coulter). The cells were grown for days and GFP expression evaluated by FACS.

RPE cells containing the GFP-RFP-sgRNA or GFP-BFP-sgRNA reporter cassette were electroporated with Lonza 4D 96-well electroporation system. 400 ng of the plasmid was pre-mixed with 40 pmol of the repair template on the 96 well PCR plate. RPE cells were trypsinized, washed with PBS and resuspended in the 1M electroporation buffer (5 mM KCl, 15 mM MgCl₂, 120 mM Na2HPO₄/NaH₂PO₄, pH 7.2, and 50 mM mannitol) to obtain cell density 200 000 cells per 20 ul of the buffer (ratio is for single-well reaction in the electroporation plate). 20 ul of the cell suspension was then dispensed in each well of the 96 well plate containing pre-mixed plasmids and repair template, gently mixed and transferred to the 96 well electroporation plate (Lonza). Electroporation was conducted using pulse code EA-104. Instantly after electroporation, 80 μl of the pre-warmed media (DMEM, supplemented with 10% FBS, no antibiotics) were added into each well. Cells were collected and plated on 48-well plates (Costar) containing 300 μl of pre-warmed medium (DMEM, supplemented with 10% FBS, no antibiotics). Liquid handling steps were done with the Biomek i7 platform. Electroporated cells were cultured for 5 days, after which the GFP expression was evaluated by FACS.

### Cas9WT Fusion Protein Validation in Endogenous Loci (HEK293T Cells)

We chose 35 Cas9WT-DNA repair fusion constructs for validation in endogenous loci. We used four different HEK293T lines, each stably expressing one of the sgRNAs targeting ELANE, CTCF1, Ench4-1, CYBB or RNF2 genomic loci (CRISPR RNA sequences in Supplementary Table 4). Cells seeded to 24-well plates at ˜100 000 cells/well were 48transfected as described above, with the following modifications: 25 ul Opti-MEM (Thermo Fisher), 2.5 ul Fugene HD (Promega), 480 ng of Cas9 fusion plasmid and 10 pmol repair DNA. After 20 min incubation, the cells were transfected in triplicate. After 5 days of incubation, DNA was extracted with PureLink 96 Genomic DNA kit (Invitrogen) and used for droplet digital PCR.

### Droplet Digital PCR

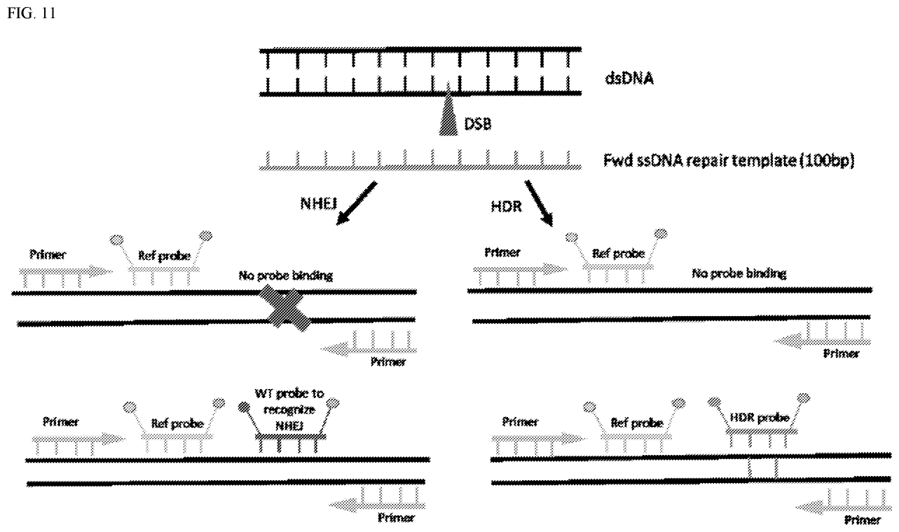

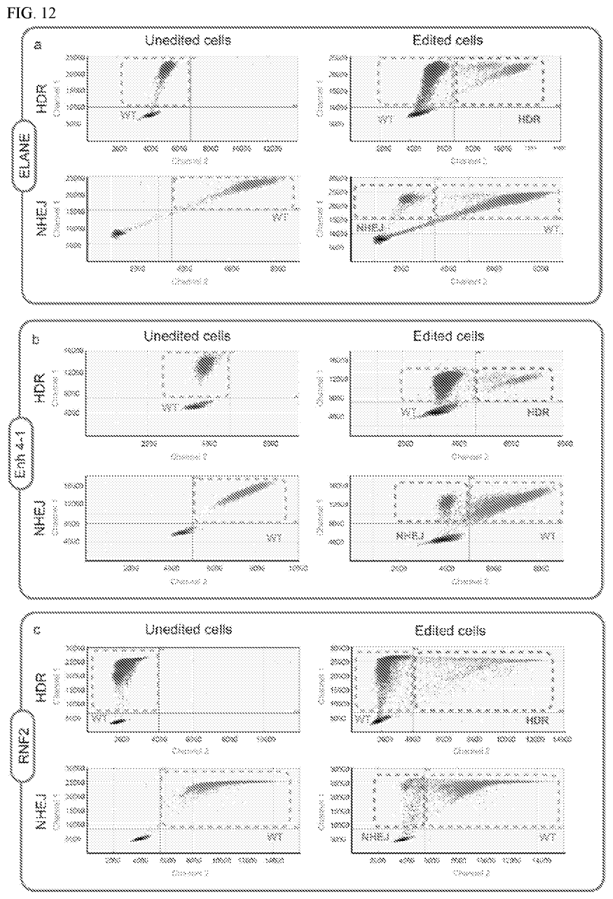

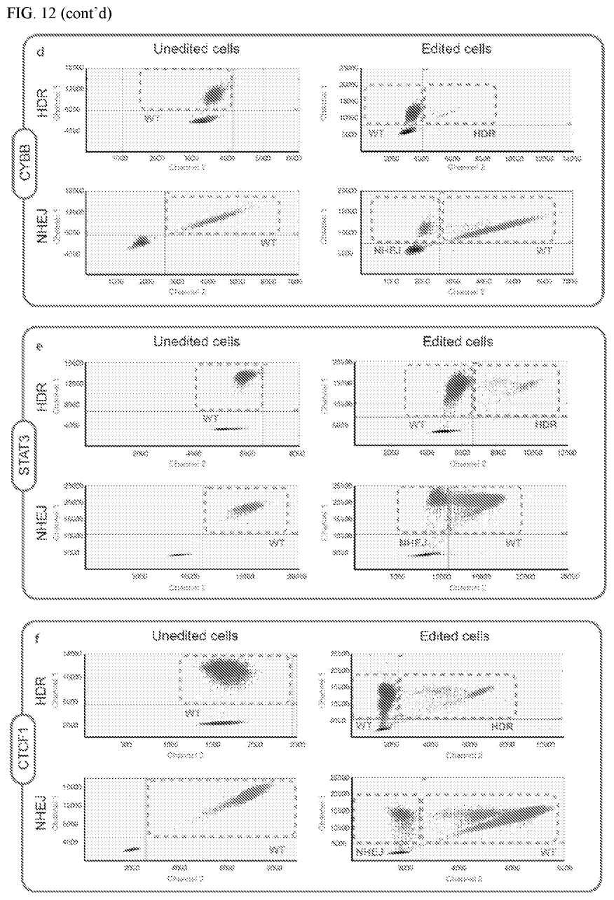

We performed droplet digital PCR (ddPCR) assays to estimate non-homologous end-joining (NHEJ) and homology directed repair (HDR) efficiencies at the RNF2, Enh4-1, FANCF, ELANE, STAT3, CTCF1 and GFP guide target loci. The assay schematic is shown in FIG. 11. The ddPCR was performed on the QX200 system (BioRad Laboratories). Final reaction mixture volume was 20 μL: 10 μL of 2×ddPCR Super Mix for Probe (BioRad Laboratories), 8 μl of DNA (concentration normalized to 8 ng/μl), primers (900 nM), reference probe (250 nM) and HDR or NHEJ probe (250 nM). Each reaction was then loaded into a sample well of an eight-well disposable cartridge (DG8™; Bio-Rad Laboratories) along with 70 μl of droplet generation oil (Bio-Rad Laboratories). Droplets were formed using a QX200™ Droplet Generator (Bio-Rad Laboratories). Droplets were transferred to a 96-well PCR plate, heat-sealed with foil and amplified using a conventional thermal cycler. The thermocycling protocol was the following: 1) 95° C.-10 min, 2) 94° C.-30 sec, 56° C.-3 min, step repeated 42 times 3) 98° C.-10 minutes 4) 4° C.-hold. The resulting PCR products were loaded on a QX200 Droplet Reader (Bio-Rad Laboratories), and the data analyzed using QuantaSoft™ software (Bio-Rad Laboratories). Primer and probe sequences are listed in Supplementary Table 4. The data was analyzed with Quantasoft software.

### Affinity-Purification Mass Spectrometry and BioID Proximity Labelling Cell Lines, Culture and Harvest

The hit Cas9 fusion proteins were synthesized and cloned to pDNOR221 Gateway entry vector (Invitrogen) by GeneArt Inc. The sequences are listed in DNA_repair_domain_library.xlsx. The inserts were cloned to MAC-Tag-C vector³ (PMID 29568061, Addgene ID #108077), which adds a c-terminal MAC tag (contains Strep-tag and modified minimal biotin ligase) to the Cas9 fusion.

For generation of the stable cell lines inducibly expressing the MAC-tagged versions of the baits, Flp-In™ T-REx™ 293 cell lines (Invitrogen, Life Technologies, R78007) were first transduced with the lentivirus containing the GFP-BFP-sgRNA cassette, as described in 1. The cells were co-transfected with the MAC-tagged expression vector and the pOG44 vector (Invitrogen) using the Fugene HD transfection reagent (Promega). Two days after transfection, cells were selected in 50 μg/ml streptomycin and hygromycin (100 μg/ml) for 2 weeks.

We tested the functionality of the GFP-BFP-sgRNA reporter cassette prior experiment as described in 3. Each stable cell line was expanded to 80% confluence in 20×150 mm cell culture plates. 2×5 plates were used for AP-MS approach, in which 1 μg/ml tetracycline was added for 30h induction, and 2×5 plates for BioID approach, in which in addition to tetracycline, 50 μM biotin was added for 30 h before harvesting. Cells from 5×150 mm fully confluent dishes (˜5×10⁷ cells) were pelleted as one biological sample. Thus, each bait protein has two biological replicates in two different experiments. All samples from one experiment were processed in parallel. Samples were snap frozen and stored at −80° C. Analyses of the baits were performed as two biological replicates.

### Affinity Purification of the Interacting Proteins

For Affinity Purification Mass Spectrometry (AP-MS) approach, the sample was lysed in 3 ml of lysis buffer 1 (0.5% IGEPAL, 50 mM Hepes, pH 8.0, 150 mM NaCl, 50 mM NaF, 1.5 mM NaVO3, 5 mM EDTA, supplemented with 0.5 mM PMSF and protease inhibitors; Sigma).

For BioID approach, Cell pellet was thawed in 3 ml ice-cold lysis buffer 2 (0.5% IGEPAL, 50 mM Hepes, pH 8.0, 150 mM NaCl, 50 mM NaF, 1.5 mM NaVO3, 5 mM EDTA, 0.1% SDS, supplemented with 0.5 mM PMSF and protease inhibitors; Sigma). Lysates were sonicated and treated with benzonase.

Cleared lysate was obtained by centrifugation and loaded consecutively on spin columns (Bio-Rad) containing lysis buffer 1, prewashed 200 μl Strep-Tactin beads (IBA, GmbH). The beads were then washed 3×1 ml with lysis buffer 1 and 4×1 ml with wash buffer (50 mM Tris-HCl, pH 8.0, 150 mM NaCl, 50 mM NaF, 5 mM EDTA). Following the final wash, beads were then resuspended in 2×300 μl elution buffer (50 mM Tris-HCl, pH 8.0, 150 mM NaCl, 50 mM NaF, 5 mM EDTA, 0.5 mM Biotin) for 5 mins and eluates collected into Eppendorf tubes, followed by a reduction of the cysteine bonds with 5 mM Tris(2-carboxyethyl)phosphine (TCEP) for 30 mins at 37° C. and alkylation with 10 mM iodoacetamide. The proteins were then digested to peptides with sequencing grade modified trypsin (Promega, V5113) at 37° C. overnight. After quenching with 10% TFA, the samples were desalted by C18 reversed-phase spin columns according to the manufacturer's instructions (Harvard Apparatus). The eluted peptide sample was dried in vacuum centrifuge and reconstituted to a final volume of 30 μl in 0.1% TFA and 1% CH₃CN.

### Liquid Chromatography-Mass Spectrometry (LC-MS)

Analysis was performed on a Q-Exactive mass spectrometer using Xcalibur version 3.0.63 coupled with an EASY-nLC 1000 system via an electrospray ionization sprayer (Thermo Fisher Scientific). In detail, peptides were eluted and separated with a C18 precolunm (Acclaim PepMap 100, 75 μm×2 cm, 3 μm, 100 A, Thermo Scientific) and analytical column (Acclaim PepMap RSLC, 75 μm×15 cm, 2 μm, 100 A; Thermo Scientific), using a 60 min buffer gradient ranging from 5 to 35% buffer B, followed by a 5 min gradient from 35 to 80% buffer B and 10 min gradient from 80 to 100% buffer B at a flow rate of 300 nl min-1 (buffer A: 0.1% formic acid in 98% HPLC grade water and 2% acetonitrile; buffer B: 0.1% formic acid in 98% acetonitrile and 2% water). For direct LC-MS analysis, 4 ul peptide samples were automatically loaded from an enclosed cooled autosampler. Data-dependent FTMS acquisition was in positive ion mode for 80 min. A full scan (200-2000 m/z) was performed with a resolution of 70,000 followed by top10 CID-MS2 ion trap scans with resolution of 17,500. Dynamic exclusion was set for 30 s.

Acquired MS2 spectral data files (Thermo.RAW) were searched with Proteome Discoverer 1.4 (Thermo Scientific) using SEQUEST search engine of the selected human component of UniProtKB/SwissProt database (www.uniprot.org/, version 2015-09). The following parameters were applied: Trypsin was selected as the enzyme and a maximum of 2 missed cleavages were permitted, precursor mass tolerance at +15 ppm and fragment mass tolerance at 0.05 Da. Carbamidomethylation of cysteine was defined as a static modification. Oxidation of methionine and biotinylation of lysine and N-termini were set as variable modifications. All reported data were based on high-confidence peptides assigned in Proteome Discoverer with FDR<1%.

### Mass Spectrometry Data Analysis and Filtering

The mass spectrometric data was searched with Proteome Discover 1.4 (Thermo Scientific) using SEQUEST search engine against the UniProtKB/SwissProt human proteome (http://www.uniprot.org/, version 2015-09). Search parameters were set either as in PMID: 29568061(QE runs; BioID)³ or in PMID: 28054750 (Orbitrap Elite runs; AP-MS)⁴. All data was filtered to medium- (AP-MS) or high-confidence (BioID) peptides according to Proteome Discoverer FDR 5% or 1%, respectively. The lists of identified proteins were conventionally filtered to remove proteins that were recognized with less than two peptides and two PSMs. The high-confidence interactors were identified using SAINT and CRAPome as in Liu et al., 2018. Each sample's abundance was normalized to its bait abundance. These bait-normalized values were used for data comparison and visualization.

### Cas9WT Fusion Plasmid Titration in RPE1 Reporter Cells

The Cas9 fusions that showed a trend for improving over Cas9 WT were Sanger sequenced to validate construct identity. We then prepared new minipreps from the original constructs using Qiaprep spin Miniprep kit (Qiagen) according to manufacturer's instructions.

RPE-GFP-BFP-gRNA cells were electroporated with Lonza 4D 16-well electroporation system as described in 4.1 with the following modifications: 200 000 cells per each well in 24 well plates; 100, 200 and 400 ng of plasmid, 40 pmol of GFP repair template; pulse code changed to EA-104. FACS analysis was performed after 5 days by iQue screener plus (Intellicyt) and analysed using Kaluza v 2.1 software (Beckman Coulter), as described in 3. (FIG. 10)

### Cas9-POLD3 in BJ-5Ta Endogenous Genes Loci

Cas9-POLD3 and Cas9WT mRNA were prepared with HiScribe™ T7 ARCA mRNA Kit with tailing (NEB-bionordika), according to the manufacturer's instructions. Total of 8000 ng stock plasmid was digested with 2 ul FastDigest MssI enzyme in the supplemented restriction-digestion buffer, total reaction volume 20 ul. Incubation carried out at 37C overnight. Length of the digested product checked by gel electrophoresis. For the IVT reaction, 1000 ng of the linearised plasmid was mixed with 10 ul of 2×ARCA/NTP mix and 2 ul of T7 RNA Polymerase mix. Reaction incubated 30 min at 37C. Sequentially, 2 ul of DNAse enzyme was added and the mixture was incubated at 37C for 15 min. Poly(A) tailing step was performed by adding 20 ul of milliQ (RNAse free), 5 ul of 10×PolyA polymerase reaction buffer and 5 ul of 10×PolyA polymerase directly to the IVT reaction, incubation at 37C for 30 min. mRNA was purified using LiCl solution, as described in the manufacturer's protocol. Aliquots were frozen in −80° C. for future use.

BJ-5ta-sgRNA cell lines were electroporated with Lonza 4D 16-well electroporation system as described in 4.1, with following modifications: 1000 ng of mRNA and 100 pmol of the corresponding HDR repair template per well, cell density-1000 000 of cells per 20 μl of the electroporation buffer. Cells were pulsed using code CA-137. Cell culture carried out on 6 well plates for 5 days, followed by the DNA isolation (DNeasy Blood & Tissue Kit (250), Qiagen). Editing efficiency evaluated by ddPCR as described in 5.

Experiments with Cas9 Fusion Proteins & Cell Cycle Timer

### Recombinant Protein Production

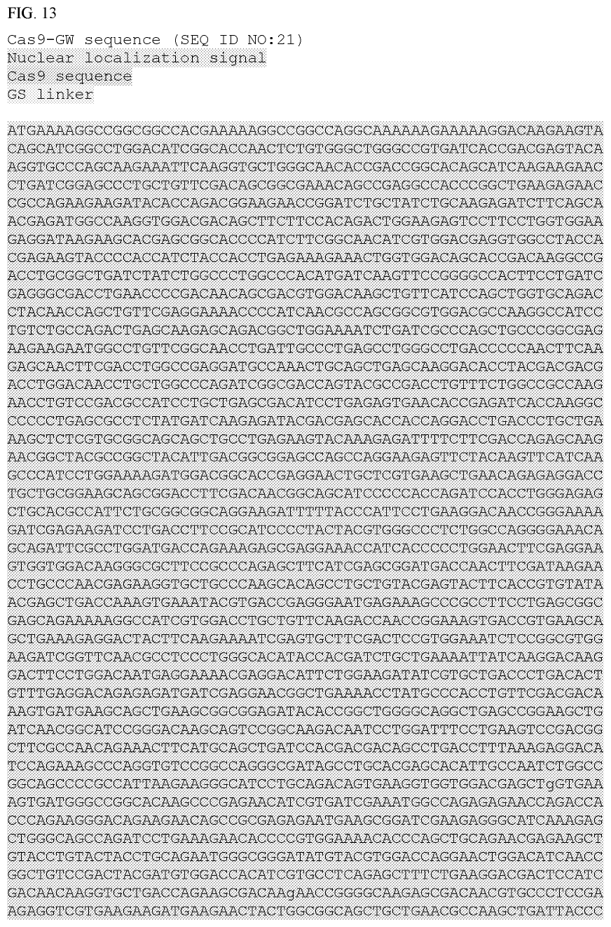

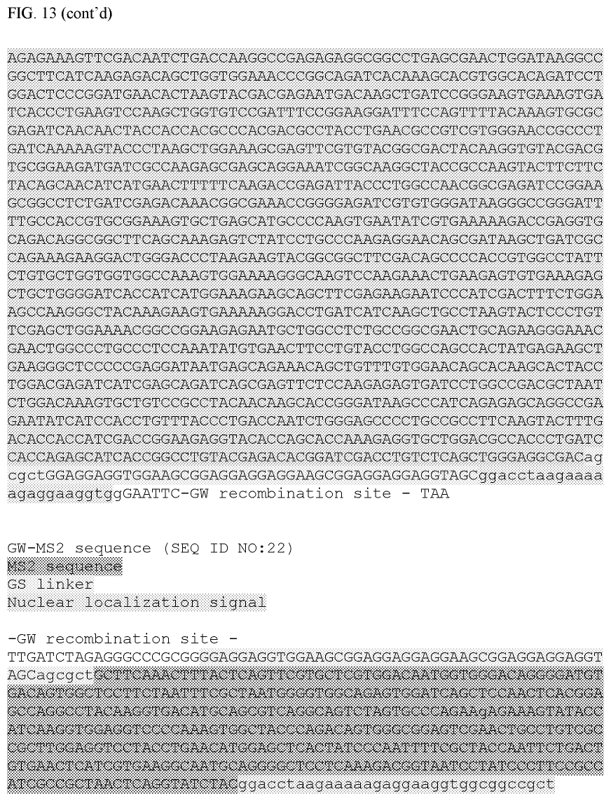

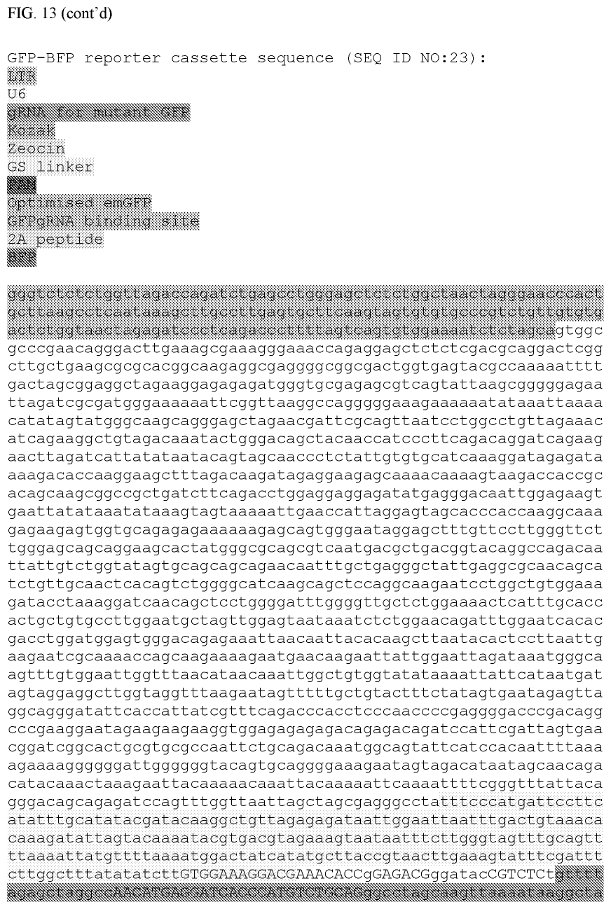

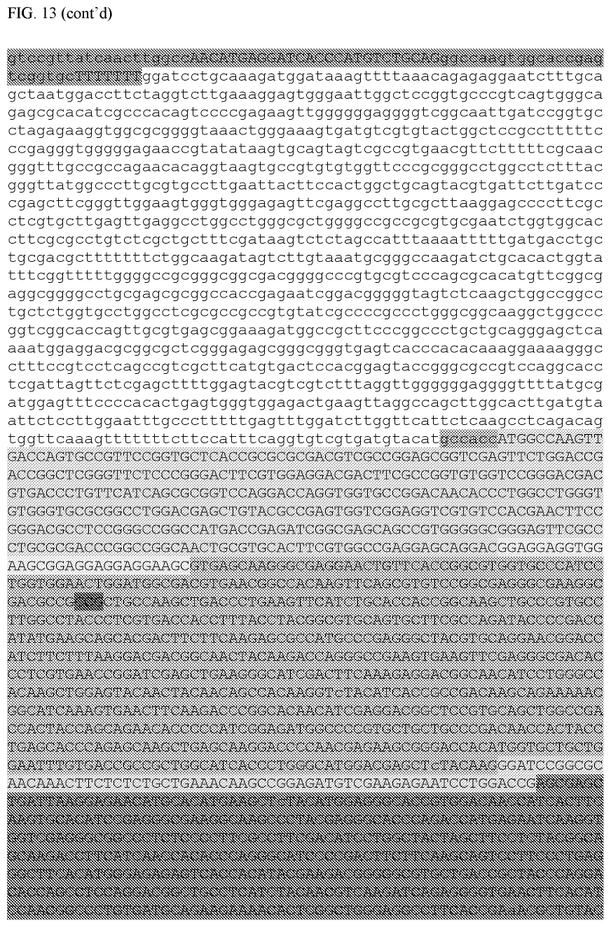

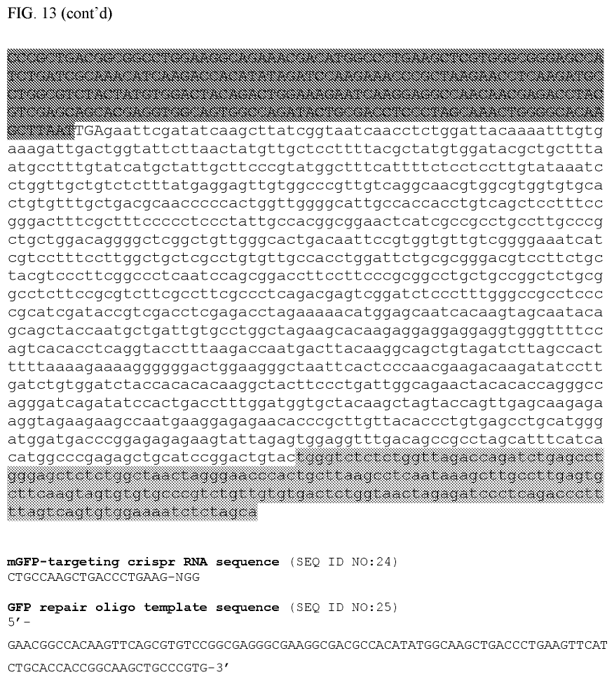

The hit Cas9 fusion proteins as well as the AcrIIA2-cdt1 fusion protein were synthesized by GeneArt Inc. and cloned to pET301/CT-DEST vector (Invitrogen) or pTH21 vector⁵ (PMID 16150509) for E. coli expression. The Cas9 sequence as well as the sequence and position of nuclear localization signal and GS linker were similar to the Cas9-GW sequence presented in FIG. 13.

All proteins contained C-terminal His-tags and were expressed in E. coli BL21 (DE3) T1R pRARE2 at 18° C. and purified by the Protein Science Facility (PSF) at Karolinska Institutet, Stockholm. Purification was performed using the HisTrap HP column (GE Healthcare) followed by gel filtration step with a HiLoad 16/60 Superdex 200 (GE Healthcare). Purity of the protein preparation was examined using SDS-PAGE followed by Commassie staining. All purified proteins were concentrated, aliquoted and stored in a storage buffer (20 mM HEPES, 300 mM NaCl, 10% glycerol, 2 mM TCEP, pH 7.5) at −80° C.

### The AcrIIA2-Cdt1 Fusion Protein Sequence

Amino acid sequence (SEQ ID NO: 19):
DVNGVTYYINIVETNDIDDLEIATDEDEMKSGNQEIILKSELKGGGGSG
GGGSSPARPALRAPASATSGSRKRARPPAAPGRDQARPPARRRLRLSVD
EVSSPSTPEAPDIPACPSPGQKIKKSTPAAGQPPHLTSAQDQDTI
DNA sequence (SEQ ID NO: 20):
ATGTTAATGGCGTGACCTACTATATTAACATCGTGGAAACCAACGATAT
CGACGATCTGGAAATTGCAACCGATGAAGATGAAATGAAAAGCGGCAAC
CAAGAGATTATCCTGAAAAGCGAACTGAAAGGTGGTGGTGGTAGCGGTG
GTGGCGGTTCATCACCGGCACGTCCGGCACTGCGTGCACCGGCAAGTGC
AACCAGCGGTAGCCGTAAACGTGCCCGTCCGCCTGCAGCACCGGGTCGT
GATCAAGCACGTCCGCCAGCACGTCGTCGTCTGCGTCTGAGCGTTGATG
AAGTTAGCAGCCCGAGTACACCGGAAGCACCGGATATTCCGGCATGTCC
GAGTCCGGGTCAGAAAATCAAAAAATCAACACCGGCAGCAGGTCAGCCT
CCGCATCTGACCAGCGCACAGGATCAGGATACAATT

### RNP Complex Preparation & Transfection

We obtained the mGFP-targeting CRISPR and TRACR RNAs from Integrated DNA Technologies. The CRISPR/TRACR hybridization and ribonucleoprotein (RNP) complex formation were done according to manufacturer's instructions. For transfection, reporter HEK293T and RPE1 cells were plated in 48-well plates at 100 000 cells/well and reverse transfected with 13 pmol of RNP and 6 pmol of repair DNA template using the CRISPRmax transfection reagent (Thermo Scientific) according to manufacturer's instructions. For the detection of corrected GFP (see 1.2), the transfected cells were cultured for 4 days, trypsinized, resuspended in warm culture medium and immediately subjected to FACS. The data was acquired by CyAn II flow cytometer (Beckman Coulter) coupled with Hypercyte robotics (Intellicyt) and analyzed with Kaluza v.1.2 (Beckman Coulter).

For Cas9WT co-transfection with AcrIIA2-Cdt1 fusion protein, we made the mGFP-targeting Cas9 RNP complexes according to manufacturer's instructions. Thereafter, the AcrIIA2-Cdt1 was added on top of the complex in 2:1 pmol ratio (i.e., 26pmol of AcrIIA2 to 13pmol of CRISPR-Cas9 RNP) and the reaction incubated in RT for 15 mins. Thereafter, the complex was transfected to RPE1 p53^{+/+} and p53^{-/-} reporter cells⁴ using CRISPRMAX transfection reagent (Thermo Scientific) per manufacturer's instructions.

### Co-Transfection of Cell Cycle Timer & Cas9 Plasmids

RPE1 reporter cells were electroporated with Lonza 4D 16-well electroporation system as described above with the following modifications: 200 000 cells per well in 24 well plates; 400 ng of Cas9WTStrep plasmid was mixed with 40 pmol of GFP repair template and 12.5, 25, 50 and 100 pmol of AcrIIA2-Cdt1 protein. Pre-mix incubated for 10 minutes; pulse code EA-104. FACS analysis was performed after 5 days of incubation by iQue screener plus (Intellicyt) and analysed using Kaluza software v.2.1 (Beckman Coulter).

### Statistics

### High-Throughput Screening

Two high-throughput screens were used:

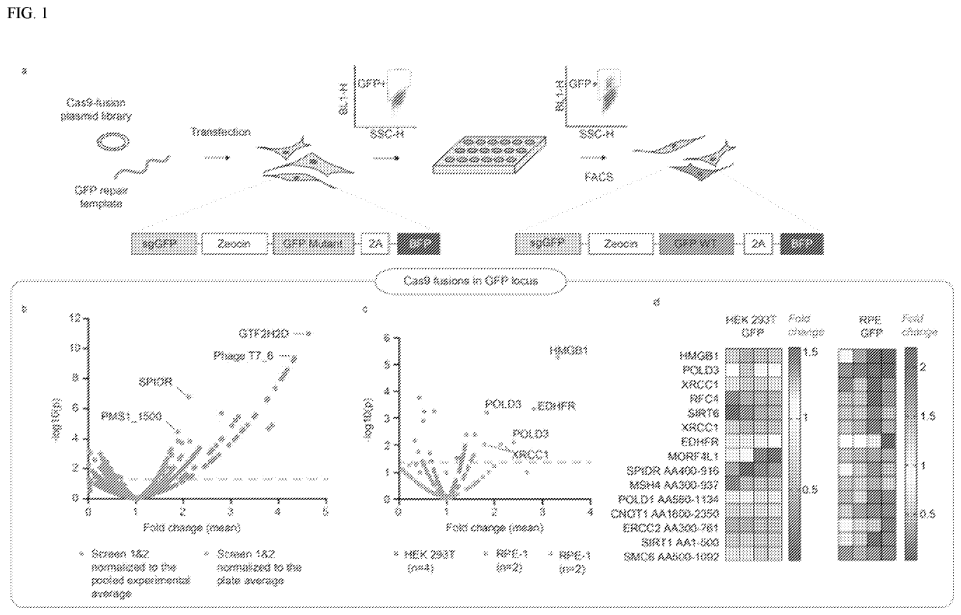

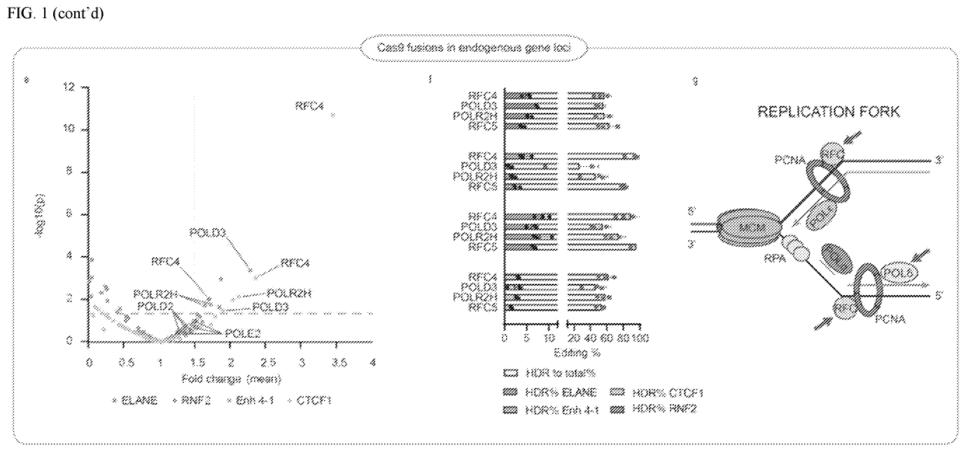

1) A screen with ˜450 DNA repair proteins fused with Cas9 and tested in clonal HEK293T cells containing the GFP-BFP-sgRNA. The screen contained three biological replicates (=parallel cell culture wells in different culture plates) and was conducted twice (n=2×3) (FIG. 1b).

2) A screen with ˜196 DNA repair proteins bound via MS2 linker with Cas9 and tested in clonal HEK293T cells containing the GFP-BFP-sgRNA. The screen contained three biological replicates (=parallel cell culture wells in different culture plates) and was conducted once (n=1×3) (FIG. 1).

To calculate statistical significance, we first calculated the average of GFP+ cells in each biological replica. The data points were then compared to the combined mean of GFP+ cells in the whole screen (“experiment average”) or the 48-well cell culture plate (“plate average”), as indicated in FIG. 1b. We compared the mean of each replicate group to the combined mean of all other replicate groups using one-way Anova testing. The statistical parameters for these experiments are shown in Supplementary Table 5. Note that, unlike pairwise comparisons in usual Anova with multiple treatments, in this case there is no multiple comparison problem since we compare in-turn the mean of each replicate group to a very large background group. Hence, each test statistic is dominated by the smaller replicate group so that the test samples are essentially disjoint.

For visualization, the data points were normalized to the average of GFP+ cells in the whole screen (“experiment average”) or the 48-well cell culture plate average (“plate average”), as indicated in FIG. 1b.

### Validation

FIG. 1c-d: We chose 60 Cas9WT-DNA repair protein fusions that performed well in the main screen, and tested them in clonal HEK293T cells containing the GFP-BFP-sgRNA, with each tested fusion having 4 replicates (parallel cell culture wells in different 48-well plates). In addition, we tested the fusions in clonal RPE-GFP-RFP-sgRNA cells once (n=1×2) and in clonal RPE-GFP-BFP-sgRNA cells once (n=1×2). We used different clonal lines than that in the major screen to account for clone-specific phenomena.

FIG. 1e-f: We chose 35 Cas9WT-DNA repair protein fusions that performed well in the main screen and in the first validation, and tested them in pooled HEK293T cells containing guides against the endogenous loci (ELANE, RNF2, Enh 4-1, CTCF1). Each tested fusion had 3 replicates (parallel cell culture wells in different 48-well plates) tested in four independent experiments (one independent experiment for each gene locus). For both validation experiments, the statistics were calculated similarly to the main screens.

### AP-MS Data

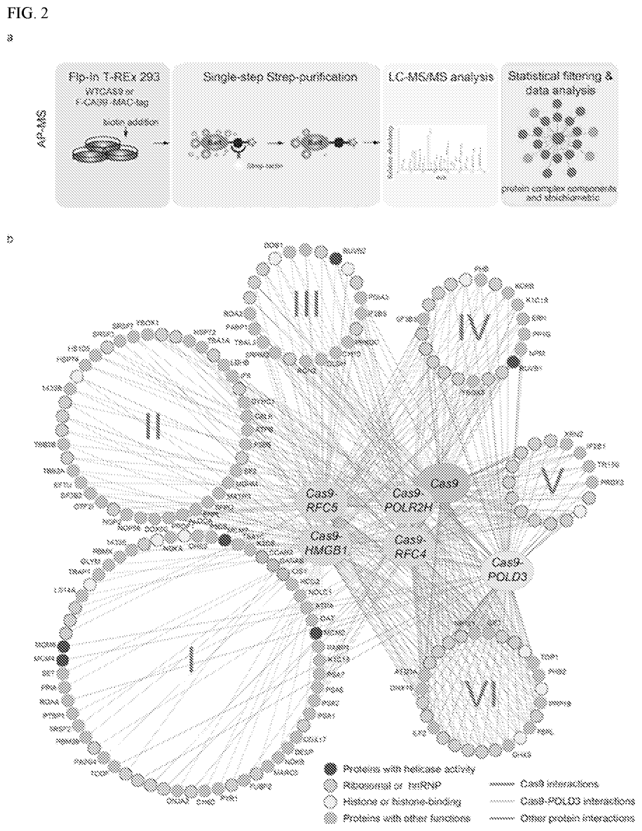

FIG. 2a-b: The mass spectrometry experiments were conducted in biological duplicates (=parallel cell culture dishes). The data analysis is explained in section 6.4.

### Smaller Experiments

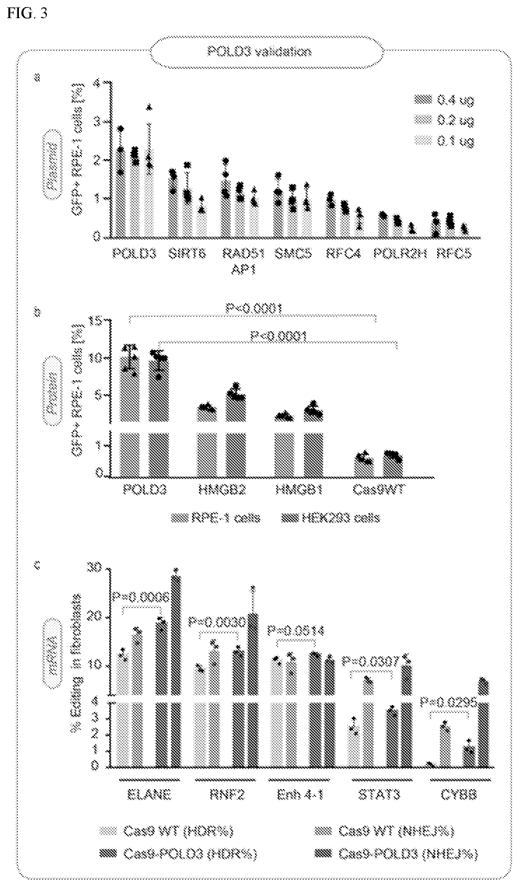

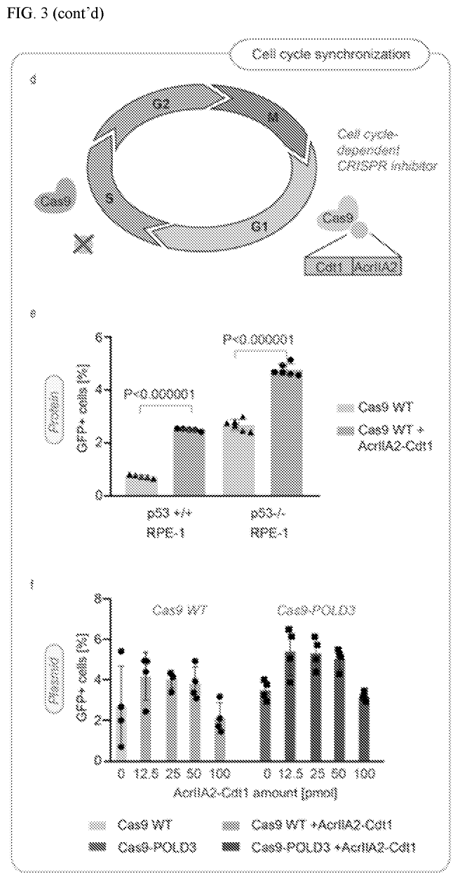

FIG. 3a: Four replicates (=parallel wells in a cell culture plate) for each condition (n=1×4), one independent experiment.

FIG. 3b: Five replicates (=parallel wells in a cell culture plate) for each condition (n=1×5), one independent experiment. One-way Anova test, adjusted for multiple comparisons (Tukey).

FIG. 3c: Triplicates (=parallel wells in a cell culture plate) for each condition (n=1×3), from two independent experiments. Unpaired two-sided Student's t-test was applied to calculate a statistical significance.

FIG. 3e: Six replicates (=parallel wells in a cell culture plate) for each condition (n=6), two independent experiments, statistical significance calculated with unpaired two-sided Student's t-test.

FIG. 3f: Four replicates (=parallel wells in a cell culture plate) for each condition (n=1×4), one independent experiment.

### METHODS REFERENCES

1. Sanjana, N. E., Shalem, O. & Zhang, F. Improved vectors and genome-wide libraries for CRISPR screening. Nature methods 11, 783 (2014).
2. Dave, K. et al. Mice deficient of Myc super-enhancer region reveal differential control mechanism between normal and pathological growth. Elife 6, e23382 (2017).
3. Liu, X. et al. An AP-MS-and BioID-compatible MAC-tag enables comprehensive mapping of protein interactions and subcellular localizations. Nature communications 9, 1-16 (2018).
4. Haapaniemi, E., Botla, S., Persson, J., Schmierer, B. & Taipale, J. CRISPR-Cas9 genome editing induces a p53-mediated DNA damage response. Nature Medicine 24, 927-930 (2018).
5. Van Den Berg, S., Löfdahl, P.-Å., Härd, T. & Berglund, H. Improved solubility of TEV protease by directed evolution. Journal of biotechnology 121, 291-298 (2006).

### Results

CRISPR/Cas9 induces customized DNA double-strand breaks (DSBs) that are commonly repaired by non-homologous end-joining (NHEJ), which leads to gene disruption and knockout. The less common pathways utilize homology-directed repair (HDR), which can induce custom genetic changes to the DSB sites by using an exogenous DNA as a template. HDR is mostly confined to the synthesis (S) phase of the cell cycle¹. Therefore, its efficiency varies between cell types² and improves upon rapid cell proliferation³, whereas factors that impair cell cycle progression from G1 to S decrease CRISPR-Cas9 mediated HDR⁴. HDR can be promoted by increasing the local concentration of the repair template⁵, and by local⁶ and general⁷ NHEJ inhibition. Overall genome editing is most efficient in open chromatin⁸ and when Cas9 is bound to an actively transcribed DNA strand where the approaching polymerase can rapidly remove the complex^{9,10,11}. Cas9 binds to the DNA for extended periods^{10,11}, and successful cut/repair requires the activation of pathways typically involved in resolving stalled DNA replication forks¹².

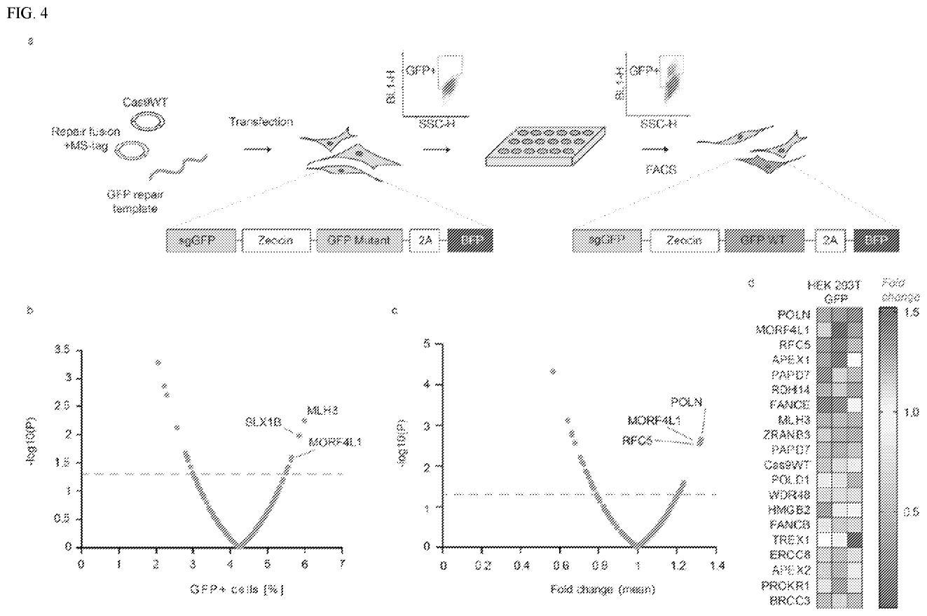

We sought to better understand the processes that govern the choice and efficiency of DNA repair in CRISPR-Cas9 induced breaks. To this end, we conducted arrayed screens where we fused ˜450 human proteins and protein domains involved in DNA repair to the C-terminus of wildtype S. pyogenes Cas9 (Cas9WT, FIG. 1A-B). In parallel, we co-expressed Cas9WT with ˜196 DNA repair proteins that were fused to the MS2 aptamer binding protein (FIG. 4). MS2 binds to modified RNA loops in the Cas9-guide complex, recruiting the MS2 fusion protein to the sgRNA/Cas9 complex. We transfected the plasmids expressing the fusion proteins along with a DNA repair template into a clonal HEK293T cell line expressing a non-functional Green Fluorescent Protein (GFP). The proportion of cells that turned GFP positive was used as a proxy for HDR efficiency.

The best MS2-coupled DNA repair proteins increased HDR efficiency by ˜40% when compared to experiment average (FIG. 4B-D). Comparison to experiment average is a common practice in CRISPR screens, as it aids to better account for technical variability between the tested conditions than comparison to Cas9WT only. The MS2-coupled proteins partially overlapped with proteins that improved editing when fused to Cas9WT. The best Cas9WT fusions improved GFP repair>2.5 times above the experiment average. We focused our further study on direct WTCas9 fusions, as coupling proteins directly to Cas9 provides means to control the DNA damage response locally at the cut site.

After the initial screen, we chose to validate Cas9WT fusions with >4% GFP+ cells per well or HDR improvement>2 times relative to experiment average (tot 60 fusions from two independent screens) (FIG. 1B and Online Methods). We transfected the fusions to clonal reporter HEK293T and RPE1 (hTERT immortalized retinal pigment epithelium) cell lines. RPE1 has a near-normal karyotype and is the target cell type in eye gene therapy.

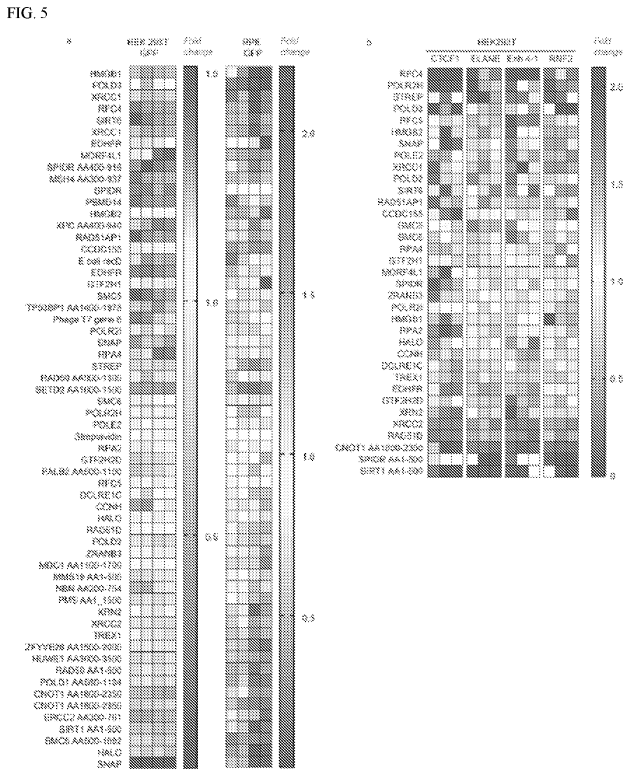

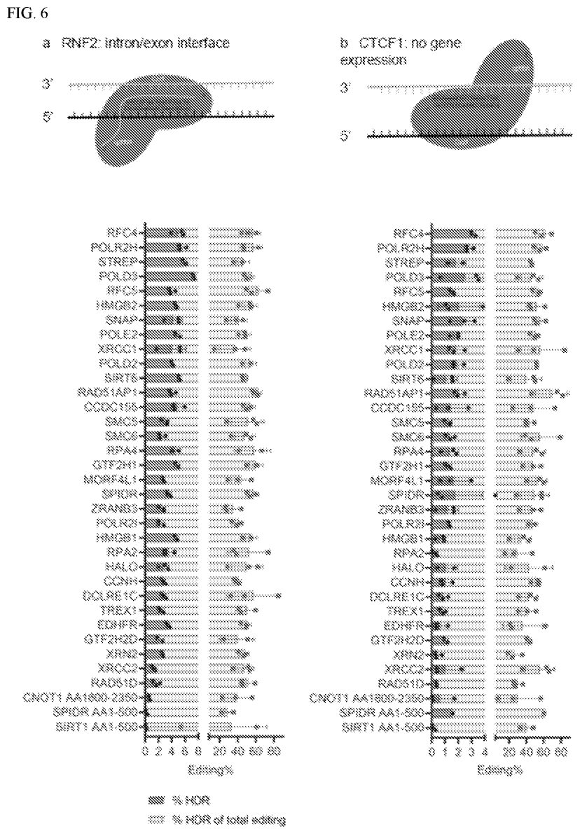

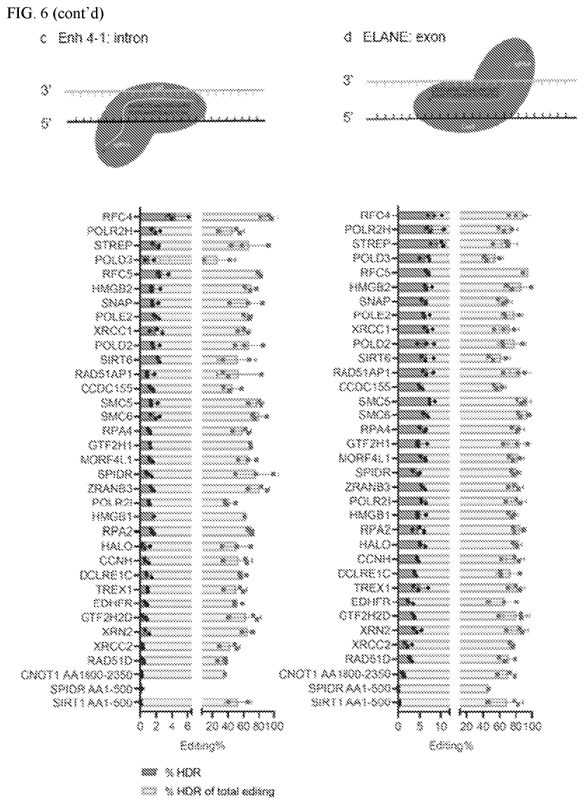

We found that in the GFP reporter locus, the proteins that improved HDR above the experiment mean were largely similar between RPE1 and HEK293T cells (FIG. 1C-D and FIG. 5A). For 35 fusions that demonstrated a stable performance in validation experiments, we also quantified the HDR and NHEJ editing by droplet digital PCR in three transcribed and one non-transcribed endogenous loci in HEK293T cells (FIG. 1E-F and FIG. 5B-3). We discovered a number of diverse polymerases among the better-performing fusions (POLD3, POLD2, POLR2H, POLE2) (FIG. 5B-3); the same trend was observed in Cas9WT co-expression screen where MS2 fusions to POLN and POLS/PAPD7 improved editing the most (FIG. 4). In addition, the replication factor C subunits RFC4 and RFC5 performed well in most of the tested loci. POLD3, POLE2, RFC4 and RFC5 are all members of the DNA replication fork machinery (FIG. 1G).

To understand how the fusion proteins modify editing, we looked at their formed molecular interactions (interactomes) using affinity-purification mass spectrometry (AP-MS) and BioID (FIG. 2A-B, Table 1). We first constructed HEK293T cell lines consisting of the GFP reporter and tet-inducible, MAC-tagged¹³ Cas9 fusions in the isogenic flip-in® locus. When Cas9 expression is induced, it complexes with the GFP-targeting guide and binds and cuts the GFP sequence. The MAC-tag in the Cas9 RNP (ribonucleoprotein) complex enables the affinity purification of the RNP complex. Additionally the MAC-tag enables proximity-dependent biotinylation of the interacting and close-proximity proteins that can be subsequently purified and identified by mass spectrometry (BioID labelling).

We mapped the stable interactions for Cas9 fusions to POLD3, RFC4, RFC5, and POLR2H, which improved editing the most across the tested loci in HEK293T cells (FIG. 2B). We also included a Cas9 fusion to HMGB1, which showed highest editing in the clonal RPE1 reporter locus. All fusions recovered a large number of RNA-binding proteins that presumably bind to the guide, as well as histones and other structural DNA components. The unique interactions of the Cas9 fusions included proteins with known nucleosome remodeling and helicase activity (Table 2). These include the minichromosome maintenance protein complex (MCM) that interacts with POLD3. Other helicases were RUVB1 and 2 that interacted with the other fusions. MCM and RUVB1-2 are helicase complexes that participate in DNA repair and replication fork stability maintenance^{14,15}. The helicases might aid editing by opening DNA and making it more accessible to Cas9 binding. After cutting, they might dislodge Cas9 from the cutting site and make the DNA ends available for processing by the DNA repair machinery^{9,10,11}.

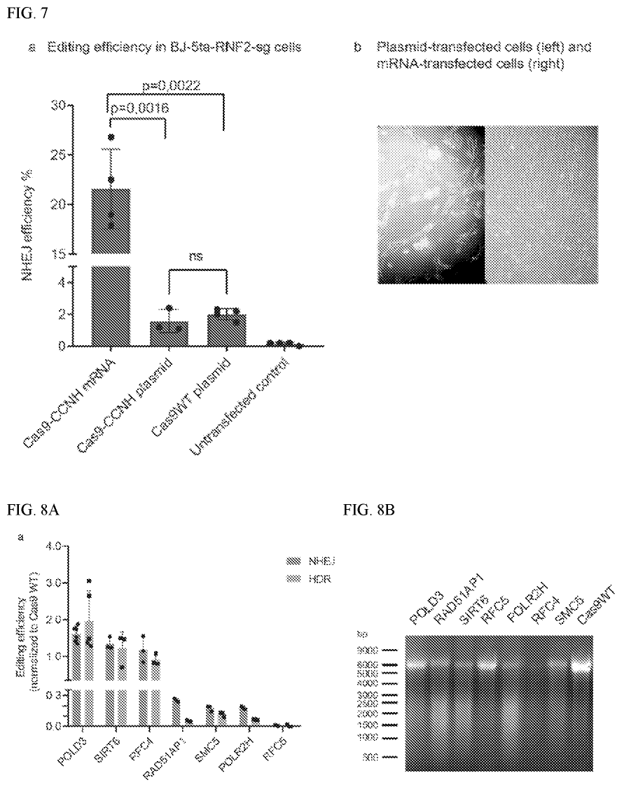

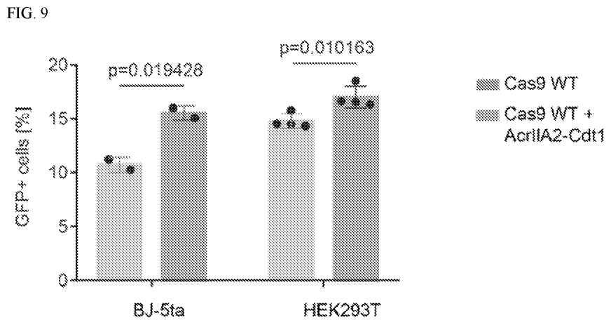

In the final validation experiments, we tested nine Cas9 fusions as plasmids, proteins and mRNA in RPE1 cells and immortalized fibroblasts. We chose the fusions that displayed high editing in RPE1 and stable performance across multiple genomic sites in HEK293T cells. First, we transfected seven fusions to RPE1 reporter cells with decreasing plasmid concentrations (FIG. 3A). Out of these, Cas9-POLD3 edited the best and was able to maintain the baseline HDR editing even at low plasmid concentrations. We also produced three Cas9 fusions proteins in E. coli and transfected them with cationic lipid transfer to RPE-1 and HEK293T cell lines. Cas9-POLD3 performed the best, although we cannot exclude differences in transfection efficiency and fusion protein folding affecting editing outcomes (FIG. 3B). Finally, we tested seven Cas9 fusions as mRNA in human hTERT immortalized fibroblasts (BJ-5ta), as these cells do not tolerate Cas9 expression from plasmid due to toxicity (FIG. 7A-B and 8A). In fibroblasts, POLD3 outperformed the other fusions and functioned across diverse genomic loci (FIG. 3C and FIG. 8A). We conclude that Cas9 fusion to POLD3 improves overall editing efficiency in diverse conditions and cell types, and additional Cas9-POLD3 linker engineering might further improve the outcome.

The HDR pathway is only effective in Synthesis (S) phase¹ of the cell cycle, and restricting CRISPR-Cas9 activity to the S cell cycle phase should increase HDR editing. Therefore, we constructed a G1-phase specific Cas9 inhibitor, which is a fusion protein consisting of a phage-derived Cas9 inhibitor AcrIIA2¹⁶ and the Cdt1 fragment from the FUCCI system¹⁷ (FIG. 3D). The Cdt1 fragment triggers degradation of AcrIIA2 when the cell enters S-phase, releasing Cas9 to be active. When co-transfected with WT CRISPR-Cas9 RNP, AcrIIA2-Cdt1 protein increased HDR by three-fold in RPE1 cells and two-fold in RPE1 cells deficient of p53, suggesting that the system is particularly beneficial for normal cells with intact DNA damage response (FIG. 3E). When tested with the Cas9-POLD3 fusion plasmid, the improvement was approximately 1.5-fold (FIG. 3F). AcrIIA2-Cdt1 protein did not improve HDR editing in HEK293T cells, likely due to their rapid proliferation and altered DNA damage response.

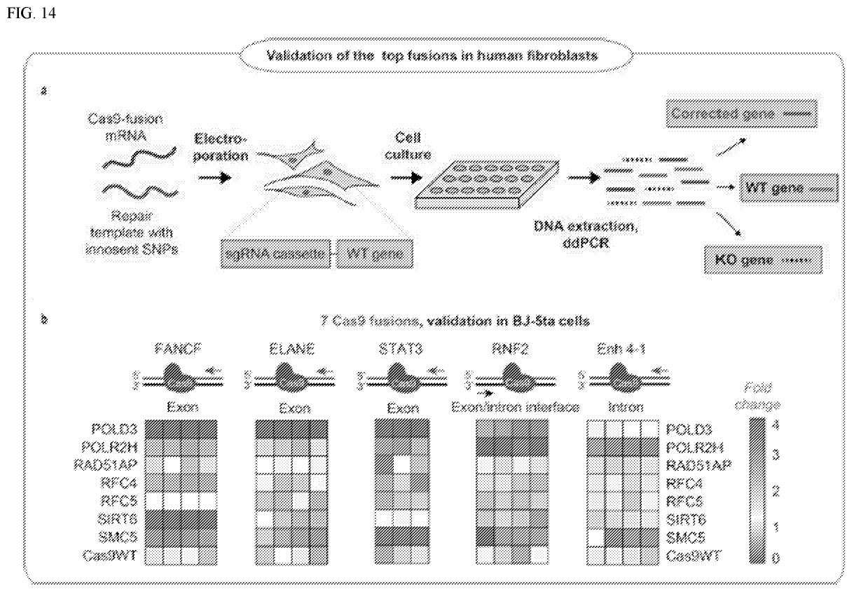

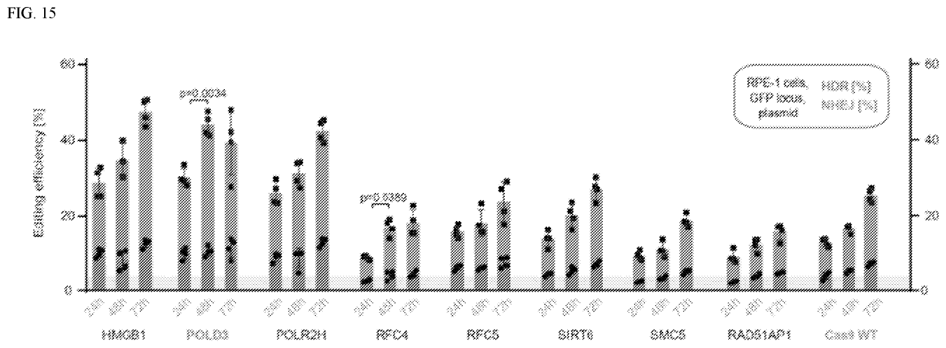

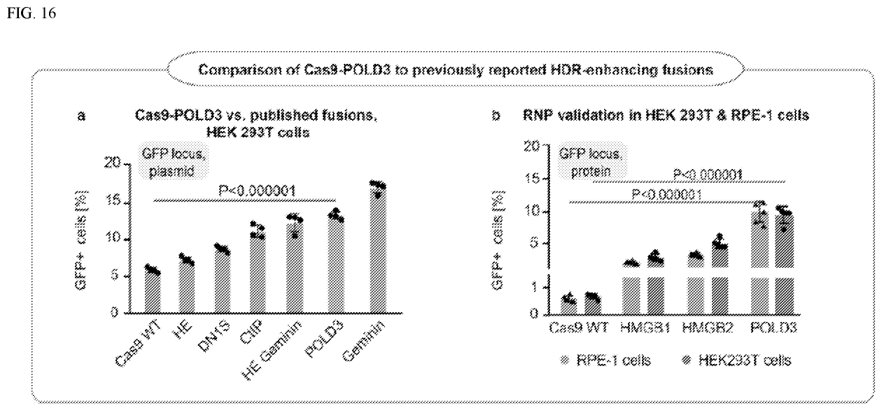

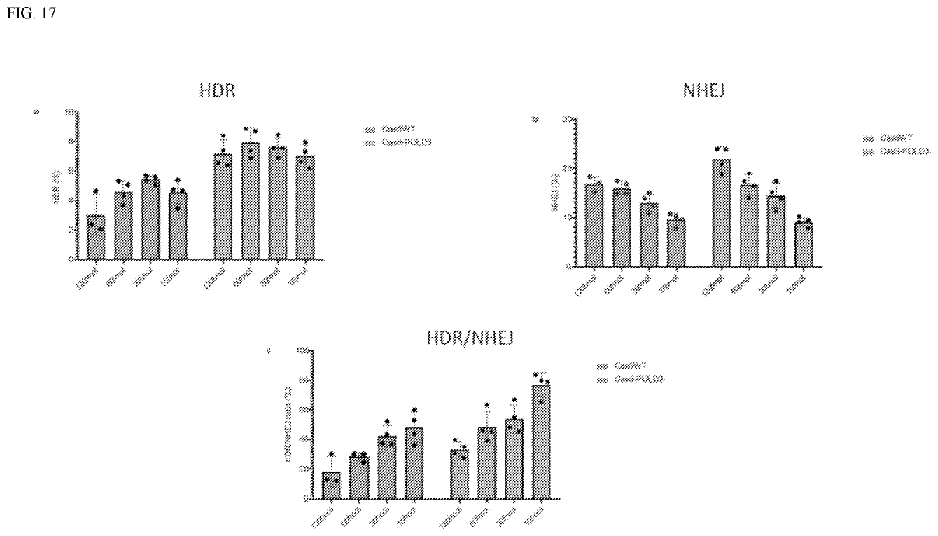

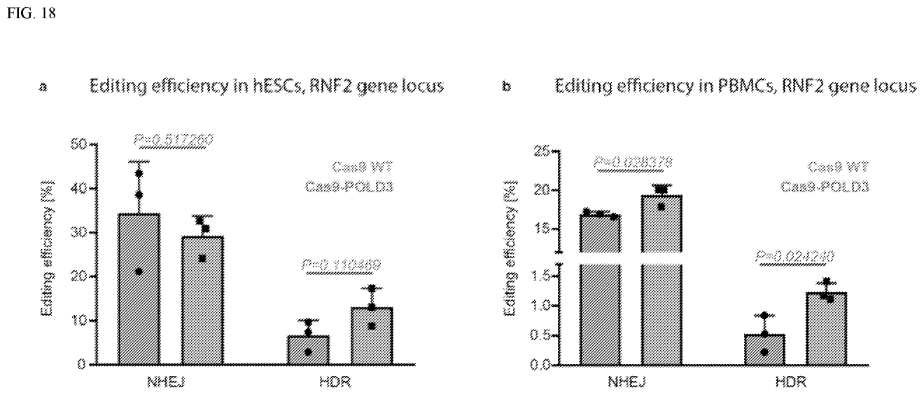

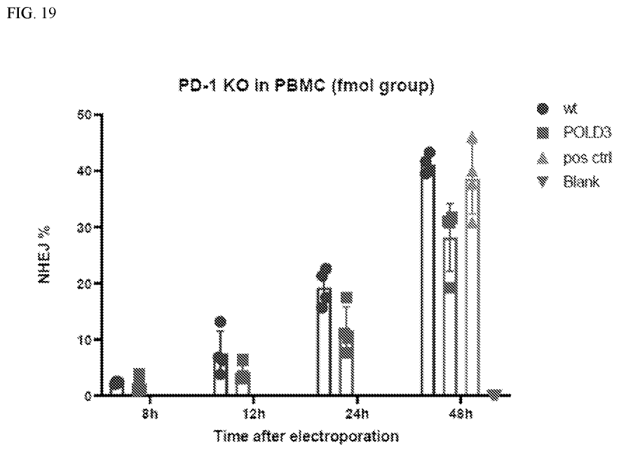

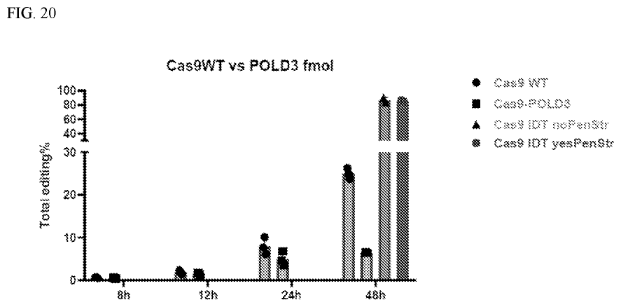

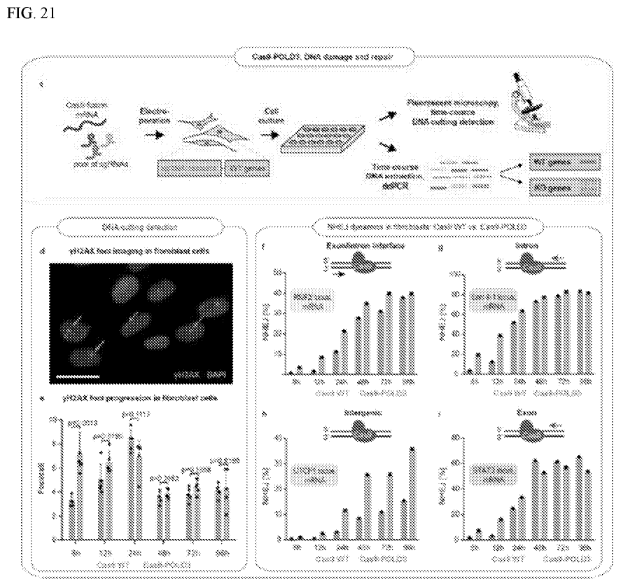

FIGS. 14-21 provide additional data with respect to the use of Cas9-POLD3 fusions as compared to wild-type Cas9 (Cas9WT). FIG. 14 illustrates the superior performance of the Cas9-POLD3 fusion over the Cas9WT in a fibroblast cell line (5 gene loci). The data presented in FIG. 15 show that Cas9-POLD3 acts faster and achieves high HDR levels at the early editing time points in comparison to the Cas9WT in RPE (Retinal Pigment Epithelium) cells (GFP locus). FIG. 16 provides a head-to-head comparison between the Cas9-POLD3 fusion and previously published HDR-improving Cas9 fusions in HEK 293T cells (GFP locus). The Cas9-POLD3 fusion generally demonstrates superior performance (with exception of Cas9-Geminin). The Cas9-POLD3 fusion shows superior levels of editing in comparison to the Cas9WT in lower molar concentrations in retinal pigment epithelium cell line, allowing to mitigate the cytotoxicity effects and reduce the potential off-target numbers. See FIG. 17. However, when tested in the primary peripheral blood mononuclear cells and human embryonic stem cell line, the Cas9-POLD3 HDR-improving effect is less prominent. See FIG. 19. Finally, the effect of the Cas9-POLD3 fusion is guide-RNA-dependent, therefore, optimization step is recommended to select the best-suited guide for each application. See FIGS. 19 and 20. FIG. 21 provides data related to time-course DNA double-strand break quantification using fluorescent microscopy and reveals that Cas9-POLD3 triggers earlier DNA break formation, as well as higher levels of DNA repair at the early time points, in comparison to the Cas9WT (Fibroblast cell line, 4 loci). Additional data (not shown) demonstrated that the Cas9-POLD3 fusion has a good safety profile with respect to off-target editing. Specifically, off-target assessment using a GUIDE-Seq method revealed that Cas9-POLD3 and Cas9WT have largely overlapping off-target profiles, with Cas9-POLD3 producing slightly fewer off-target edits (HEK 293T cells, HEK site 4 gene locus). An amplicon sequencing method further showed that on-target signatures of Cas9-POLD3 and Cas9WT are highly similar (fibroblast cell line, 2 gene loci).

Through screening 450 human proteins involved in DNA repair, we have identified a subset that can improve genome editing as WTCas9 fusions. Several of these are DNA replication fork components, which functionally interact with helicase proteins. We also note that diverse DNA and RNA polymerases improve editing when either fused to or coexpressed with Cas9. Of these, Cas9 fusion to POLD3 is the most efficient and can enhance editing in low CRISPR-Cas9 concentrations, potentially mitigating the toxicities associated with high CRISPR quantities. We further show that targeting the editing to S cell cycle phase benefits HDR outcomes. This study increases our understanding of the DNA repair pathways that participate in CRISPR-Cas9 genome editing, and provides new CRISPR tools for the scientific community.

TABLE 2
CRISPR gRNA sequences of guides that target endogenous loci:

| | | | | Guide | |
|---|---|---|---|---|---|
| | Abbre- | Location | sgRNA | binding | Gene |
| Targeted gene | viation | (map in Hg19) | sequence | strand | orientation |
| CCAT1 colon | Enh4-1 | chr8:128226403- | GTAGAATGTCA | - | REV |
| cancer associated | | 128226422 | ACTTCATGA | | |
| transcript 1 | | | (SEQ ID NO: 26) | | |
| Transcriptional | CTCF-1 | chr8:128746377- | TACTTTCGCAA | + | N/A |
| repressor CTCF | | 128746396 | ACCTGAACG | | |
| | | | (SEQ ID NO: 27) | | |
| Ring finger | RNF2 | chr1:185056770- | GTCATCTTAGT | - | FWD |
| protein 2 | | 185056789 | CATTACCTG | | |
| | | | (SEQ ID NO: 28) | | |
| Signal transducer | STAT3 | chr17:40481574- | CTCTGCAGAAT | - | REV |
| and activator of | | 40481594 | TCAAACACT | | |
| transcription 3 | | | (SEQ ID NO: 29) | | |
| Elastase, | ELANE | chr19:853291- | GAGCCCATAAC | + | FWD |
| neutrophil | | 853310 | CTCTCGCGG | | |
| expressed | | | (SEQ ID NO: 30) | | |
| Cytochrome b-245 | CYBB | chrX:37664424- | AAGTCCTTTCA | + | FWD |
| beta chain | | 37664443 | TTTATCGGA | | |
| | | | (SEQ ID NO: 31) | | |

TABLE 3
Sequences of ssDNA repair oligos, ddPCR primers and probes

| | | ddPCR | ddPCR | ddPCR | ddPCR | ddPCR |
|---|---|---|---|---|---|---|
| Targeted | | primer | primer | probe | probe | probe |
| gene | DNA repair sequence | fwd | rev | NHEJ | HDR | reference |
| Enh4-1 | ATGTGCTAGACAATATAT | TGTTGA | ACCCT | CTTGCC | CCTTGa | TGGAGGA |
| | TAGGTGTGGGATACAGT | GTGTGC | TGCCT | CTCATG | gagtcaG | CCGATCAT |
| | GAAATCAAATCCTTGagag | CTGGAT | CTTTC | AAGTTG | AAGTTG | GTGCTAGA |
| | tcaGAAGTTGACATTCTAC | (SEQ ID | CCT | ACATTC | ACATTC | CA (SEQ ID |
| | TGACAGAAGACATTCAA | NO: 33) | (SEQ ID | (SEQ ID | TA (SEQ | NO: 37) |
| | GCAATACATG (SEQ ID | | NO: 34) | NO: 35) | ID NO: | |
| | NO: 32) | | | | 36) | |
| CTCF-1 | TTTGCTGCAAAGCGTCTT | AGGTG | GGAGC | CAAACC | CGCAAA | CCTCGGA |
| | TCCCTCCGCCCCCTCTC | GCTGG | AACCA | TGAACG | CCTGTT | CGCTCCTG |
| | TGGGCAGCACttatcaaCAG | AAACTT | ATCGC | CGGGT | GATAAG | CTCCT |
| | GTTTGCGAAAGTAAAGTA | GT (SEQ | TATG | GC (SEQ | TGCTGC | (SEQ ID |
| | AGTGTGCCCTCTACTGG | ID NO: | (SEQ ID | ID NO: | (SEQ ID | NO: 43) |
| | CAGCAGAGAT (SEQ ID | 39) | NO: 40) | 41) | NO: 42) | |
| | NO: 38) | | | | | |
| RNF2 | TAAGCAAAACATGGGAA | CTTCTT | GCCAA | ACCTCA | CTAGTT | CTGTGCAG |
| | CTCAGTTTATATGAGTTA | TATTTC | CATAC | GGTAAT | CGGCT | ACAAACGG |
| | CAACGAACACCTagttcggct | CAGCAA | AGAAG | GACTAA | CCTAAG | AACTCAA |
| | cCTAAGATGACTGCCAAG | TGTCTC | TCAGG | GATGAC | ATGACT | (SEQ ID |
| | GGGCATATGAGACGTGT | (SEQ ID | (SEQ ID | TGC | GC (SEQ | NO: 49) |
| | AAACTGGGA (SEQ ID NO: | NO: 45) | NO: 46) | (SEQ ID | ID NO: | |
| | 44) | | | NO: 47) | 48) | |
| STAT3 | CCGGCAGCCAGAGGCC | GCTCCC | CCTGT | CAAGTG | TGAATT | AAAGGCA |
| | CTTTGTGAAGGGGAGCT | TCAGG | GATTC | TTTGAA | CTGCAa | GGTGTCCT |
| | CCTCCCACATAtaAAcaGT | GTCTGT | AGATC | TTCTGC | tagttCTG | GTGA (SEQ |
| | TTGAATTCTGCAatagttCT | A (SEQ | CCG | AGAGA | CCGTT | ID NO: 55) |
| | GCCGTTGTTGGATTCTTC | ID NO: | (SEQ ID | GGC | (SEQ ID | |
| | CATGTTCATC (SEQ ID | 51) | NO: 52) | (SEQ ID | NO: 54) | |
| | NO: 50) | | | NO: 53) | | |
| ELANE | GTTTTCGAAGATGCGCT | GGATG | GCACC | CTCGC | CATAAC | ACCCCGTA |
| | GCACGGCGAACACCTGC | GGGAC | TGGAG | GGCGG | CTCTCa | AACTTGCT |
| | CGGGTGGGCTCttGatGtG | GACAAG | AATCA | GAGC | CatCaaG | CAACGA |
| | AGAGGTTATGGGCTCCC | G (SEQ | CGA | (SEQ ID | AGCCC | (SEQ ID |
| | AGGACCACCCGCACCGC | ID NO: | (SEQ ID | NO: 59) | (SEQ ID | NO: 61) |
| | GCGGACGTTTCTG (SEQ | 57) | NO: 58) | | NO: 60) | |
| | ID NO: 56) | | | | | |
| CYBB | GCACTCTCAGGCCTCAC | TGATGT | CCTGT | TAAGTC | AaTAAG | ACCATGG |
| | ATGGCAGAGTATGGTTA | TAGTGG | CTCTC | CTTTCA | TCCTTg | GAAGTGAA |
| | CTGCTCTAAGGtatTaaGA | GAGCA | TGGAA | TTTATC | gATTTA | GAAAAGCT |
| | TAAATccAAGGACTTAttTT | GG (SEQ | CCAC | GGAGG | TCttAata | TC (SEQ ID |
| | TTTGAGCTTCAGATTGGT | ID NO: | (SEQ ID | GC (SEQ | CCTT | NO: 67) |
| | GGCGTTATTGCA (SEQ ID | 63) | NO: 64) | ID NO: | (SEQ ID | |
| | NO: 62) | | | 65) | NO: 66) | |

*Letters written in non-capital font in the “DNA repair sequence column” represent nucleotides that are different from the original WT gene sequence.

### REFERENCES

1. Hustedt, N. & Durocher, D. The control of DNA repair by the cell cycle. Nature cell biology 19, 1-9 (2017).
2. Miyaoka, Y. et al. Systematic quantification of HDR and NHEJ reveals effects of locus, nuclease, and cell type on genome-editing. Scientific reports 6, 23549 (2016).
3. Roth, T. L. et al. Reprogramming human T cell function and specificity with non-viral genome targeting. Nature 559, 405-409 (2018).
4. Haapaniemi, E., Botla, S., Persson, J., Schmierer, B. & Taipale, J. CRISPR-Cas9 genome editing induces a p53-mediated DNA damage response. Nature Medicine 24, 927-930 (2018).
5. Savic, N. et al. Covalent linkage of the DNA repair template to the CRISPR-Cas9 nuclease enhances homology-directed repair. Elife 7, e33761 (2018).
6. Jayavaradhan, R. et al. CRISPR-Cas9 fusion to dominant-negative 53BP1 enhances HDR and inhibits NHEJ specifically at Cas9 target sites. Nature communications 10, 1-13 (2019).
7. Maruyama, T. et al. Inhibition of non-homologous end joining increases the efficiency of CRISPR/Cas9-mediated precise [TM: inserted] genome editing. Nature biotechnology 33, 538 (2015).
8. Chen, F. et al. Targeted activation of diverse CRISPR-Cas systems for mammalian genome editing via proximal CRISPR targeting. Nature communications 8, 1-12 (2017).
9. Clarke, R. et al. Enhanced bacterial immunity and mammalian genome editing via RNA-polymerase-mediated dislodging of Cas9 from double-strand DNA breaks. Molecular cell 71, 42-55. e8 (2018).
10. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nature biotechnology 34, 339-344 (2016).
11. Jones, D. L. et al. Kinetics of dCas9 target search in Escherichia coli. Science 357, 1420-1424 (2017).
12. Richardson, C. D. et al. CRISPR-Cas9 genome editing in human cells occurs via the Fanconi anemia pathway. Nature genetics 50, 1132-1139 (2018).
13. Liu, X. et al. An AP-MS-and BioID-compatible MAC-tag enables comprehensive mapping of protein interactions and subcellular localizations. Nature communications 9, 1-16 (2018).
14. Rajendra, E., Garaycoechea, J. I., Patel, K. J. & Passmore, L. A. Abundance of the Fanconi anaemia core complex is regulated by the RuvBL1 and RuvBL2 AAA+ ATPases. Nucleic acids research 42, 13736-13748 (2014).
15. Lossaint, G. et al. FANCD2 binds MCM proteins and controls replisome function upon activation of s phase checkpoint signaling. Molecular cell 51, 678-690 (2013).
16. Rauch, B. J. et al. Inhibition of CRISPR-Cas9 with bacteriophage proteins. Cell168, 150-158. e10 (2017).
17. Grant, G. D., Kedziora, K. M., Limas, J. C., Cook, J. G. & Purvis, J. E. Accurate delineation of cell cycle phase transitions in living cells with PIP-FUCCI. Cell Cycle 17, 2496-2516 (2018).

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

What is claimed is:

1. A composition, comprising: a nucleic acid encoding a fusion protein comprising a Cas9 polypeptide fused to a nucleic acid repair protein.

2. A composition, comprising a first nucleic acid encoding a Cas9 polypeptide and a second nucleic acid encoding a nucleic acid repair protein, wherein the Cas9 polypeptide and nucleic acid repair protein are from different organisms.

3. The composition of claim 1 or 2, wherein the nucleic acid repair protein is a replicative polymerase.

4. The composition of claim 3, wherein the replicative polymerase is a DNA polymerase or an RNA polymerase.

5. The composition of claim 3 or 4, wherein the replicative polymerase is a human polymerase.

6. The composition of claims 3 to 5, wherein the DNA polymerase is a DNA polymerase delta.

7. The composition of claim 6, wherein the DNA polymerase delta is DNA polymerase delta III (POLD3).

8. The composition of claims 3 or 4, wherein the DNA polymerase is POLN.

9. The composition of claim 3 or 4, wherein the DNA polymerase is POLR2H or PAPD7.

10. The composition of claim 1 or 2, wherein the nucleic acid repair protein a DNA replication factor.

11. The composition of claim 9, wherein the DNA replication factor is rfc4 or rfc5.

12. The composition of claim 1 or 2, wherein said nucleic acid repair protein is SIRT6.

13. The composition of any of the preceding claims, wherein the nucleic acid is on a vector.

14. The composition of claim 2, wherein the first and second nucleic acid are on the same or different vectors.

15. The composition of claim 14, wherein the first and second nucleic acid are on the same vector and are separated by an internal ribosome entry site (IRES).

16. The composition of any one of claims 13 to 15, wherein the vector is a plasmid or viral vector.

17. A fusion protein encoded by the composition of any one of claims 1 and 3 to 16.

18. A pair of polypeptides encoding by the composition of claim 2.

19. A kit or system, comprising:
a) a nucleic acid, fusion protein, or pair of polypeptides of any of the preceding claims; and
b) a plurality of guide RNAs.

20. The kit or system of claim 19, wherein the plurality of guide RNAs is one or two.

21. The kit or system of claim 19 or 20, wherein the kit or system further comprises a nucleic acid encoding an exogenous gene of interest.

22. The kit or system of claim 21, wherein the exogenous gene of interest has 5′ and 3′ flanking sequences that are homologous to a target site in a chromosome in a target cell.

23. The kit or system of any one of claims 21 to 21, wherein the nucleic acid is on a vector.

24. The kit or system of claims 19 to 23, wherein the guide RNA hybridizes to an endogenous gene of interest.

25. The kit or system of any one of claims 19 to 24, wherein the guide RNA is a Single-guide RNA (sgRNA).

26. A method, comprising:
introducing a system of any one of claims 19 to 25 into a cell.

27. A gene editing method, comprising:
introducing a system of any one of claims 19 to 25 into a cell.

28. The method of claims 26 or 27, wherein the introducing results in disruption, deletion, or insertion of a target nucleic acid in the cell.

29. The method of claim 28, wherein the target nucleic acid is a gene.

30. The method of claim 29, wherein the gene editing results in an increase or decrease in expression of an endogenous or exogenous gene in the cell.

31. The method of claims 27 to 30, wherein the cell is a eukaryotic cell.

32. The method of claim 31, wherein the eukaryotic cell is a mammalian cell.

33. The method of claim 32, wherein the mammalian cell is a human cell.

34. The method of any one of claims 27 to 33, wherein the cell is in vitro, ex vivo, or in vivo.

35. The method of claim 34, wherein the method treats a disease or condition in a subject.

36. The method of any one of claims 27 to 35, wherein the gene editing comprises homology directed repair or non-homologous end joining.

37. A cell comprising the system of any one of claims 19 to 25.

38. Use of the system of any one of claims 19 to 25 to alter expression of gene in a target cell or edit the genome of a target cell.


SEQUENCE LISTING (67 sequences)

SEQ ID NO: 1 - protein, 23 aa, Artificial sequence
  synthetic
  n (9)..(22): N is A, G, T, or C
MMMMMMMMNNNNNNNNNNNNXGG

SEQ ID NO: 2 - protein, 15 aa, Artificial sequence
  synthetic
  n (1)..(12): N is A, G, T, or C
  misc_feature (13)..(13): Xaa can be any naturally occurring amino acid
NNNNNNNNNNNNXGG

SEQ ID NO: 3 - protein, 23 aa, Artificial sequence
  synthetic
  n (10)..(20): N is A, G, T, or C
  misc_feature (21)..(21): Xaa can be any naturally occurring amino acid
MMMMMMMMMNNNNNNNNNNNXGG

SEQ ID NO: 4 - protein, 14 aa, Artificial sequence
  synthetic
  n (1)..(11): N is A, G, T, or C
  misc_feature (12)..(12): Xaa can be any naturally occurring amino acid
NNNNNNNNNNNXGG

SEQ ID NO: 5 - protein, 27 aa, Artificial sequence
  synthetic
  n (9)..(20): N is A, G, T, or C
  misc_feature (21)..(22): Xaa can be any naturally occurring amino acid
  w (27)..(27): W is A or T
MMMMMMMMNNNNNNNNNNNNXXAGAAW

SEQ ID NO: 6 - protein, 19 aa, Artificial sequence
  synthetic
  n (1)..(12): N is A, G, T, or C
  misc_feature (13)..(14): Xaa can be any naturally occurring amino acid
  w (19)..(19): W is A or T
NNNNNNNNNNNNXXAGAAW

SEQ ID NO: 7 - protein, 27 aa, Artificial sequence
  synthetic
  n (10)..(20): N is A, G, T
  misc_feature (21)..(22): Xaa can be any naturally occurring amino acid
  w (27)..(27): W is A or T
MMMMMMMMMNNNNNNNNNNNXXAGAAW

SEQ ID NO: 8 - protein, 18 aa, Artificial sequence
  synthetic
  n (1)..(11): N is A, G, T
  misc_feature (12)..(13): Xaa can be any naturally occurring amino acid
  w (18)..(18): W is A or T
NNNNNNNNNNNXXAGAAW

SEQ ID NO: 9 - protein, 25 aa, Artificial sequence
  synthetic
  n (9)..(20): N is A, G, T, or C
  misc_feature (21)..(21): Xaa can be any naturally occurring amino acid
  misc_feature (24)..(24): Xaa can be any naturally occurring amino acid
MMMMMMMMNNNNNNNNNNNNXGGXG

SEQ ID NO: 10 - protein, 17 aa, Artificial sequence
  synthetic
  n (1)..(12): N is A, G, T, or C
  misc_feature (13)..(13): Xaa can be any naturally occurring amino acid
  misc_feature (16)..(16): Xaa can be any naturally occurring amino acid
NNNNNNNNNNNNXGGXG

SEQ ID NO: 11 - protein, 25 aa, Artificial sequence
  synthetic
  n (10)..(20): N is A, G, T, or C
  misc_feature (21)..(21): Xaa can be any naturally occurring amino acid
  misc_feature (24)..(24): Xaa can be any naturally occurring amino acid
MMMMMMMMMNNNNNNNNNNNXGGXG

SEQ ID NO: 12 - protein, 16 aa, Artificial sequence
  synthetic
  n (1)..(11): N is A, G, T, or C
  misc_feature (12)..(12): Xaa can be any naturally occurring amino acid
  misc_feature (15)..(15): Xaa can be any naturally occurring amino acid
NNNNNNNNNNNXGGXG

SEQ ID NO: 13 - protein, 137 aa, Artificial sequence
  synthetic
  n (1)..(20): N represents a base of a guide sequence
NNNNNNNNNNNNNNNNNNNNGTTTTTGTACTCTCAAGATTTAGAAATAAATCTTGCAGAAGCTACAAAGATAAGGCTTCATGCCGAAATCAACACCCTGTCATTTTATGGCAGGGTGTTTTCGTTATTTAATTTTTT

SEQ ID NO: 14 - protein, 123 aa, Artificial sequence
  synthetic
  n (1)..(20): N represents a base of a guide sequence
NNNNNNNNNNNNNNNNNNNNGTTTTTGTACTCTCAGAAATGCAGAAGCTACAAAGATAAGGCTTCATGCCGAAATCAACACCCTGTCATTTTATGGCAGGGTGTTTTCGTTATTTAATTTTTT

SEQ ID NO: 15 - protein, 110 aa, Artificial sequence
  synthetic
  n (1)..(20): N represents a base of a guide sequence
NNNNNNNNNNNNNNNNNNNNGTTTTTGTACTCTCAGAAATGCAGAAGCTACAAAGATAAGGCTTCATGCCGAAATCAACACCCTGTCATTTTATGGCAGGGTGTTTTTTT

SEQ ID NO: 16 - protein, 105 aa, Artificial sequence
  synthetic
  n (1)..(20): N represents a base of a guide sequence
NNNNNNNNNNNNNNNNNNNNGTTTTTGTACTCTCAGAAATAGCAAGTTAAAATAAGGCTAGTCCGTTATCAACTTGAAAAAGTGGCACCGAGTCGGTGCTTTTTT

SEQ ID NO: 17 - protein, 88 aa, Artificial sequence
  synthetic
  n (1)..(20): N represents a base of a guide sequence
NNNNNNNNNNNNNNNNNNNNGTTTTAGAGCTAGAAATAGCAAGTTAAAATAAGGCTAGTCCGTTATCAACTTGAAAAAGTGTTTTTTT

SEQ ID NO: 18 - protein, 76 aa, Artificial sequence
  synthetic
  n (1)..(20): N represents a base of a guide sequence
NNNNNNNNNNNNNNNNNNNNGTTTTAGAGCTAGAAATAGCAAGTTAAAATAAGGCTAGTCCGCTATCATTTTTTTT

SEQ ID NO: 19 - protein, 143 aa, Artificial sequence
  synthetic
DVNGVTYYINIVETNDIDDLEIATDEDEMKSGNQEIILKSELKGGGGSGGGGSSPARPALRAPASATSGSRKRARPPAAPGRDQARPPARRRLRLSVDEVSSPSTPEAPDIPACPSPGQKIKKSTPAAGQPPHLTSAQDQDTI

SEQ ID NO: 20 - DNA, 428 bp, Artificial sequence
  synthetic
atgttaatggcgtgacctactatattaacatcgtggaaaccaacgatatcgacgatctggaaattgcaaccgatgaagatgaaatgaaaagcggcaaccaagagattatcctgaaaagcgaactgaaaggtggtggtggtagcggtggtggcggttcatcaccggcacgtccggcactgcgtgcaccggcaagtgcaaccagcggtagccgtaaacgtgcccgtccgcctgcagcaccgggtcgtgatcaagcacgtccgccagcacgtcgtcgtctgcgtctgagcgttgatgaagttagcagcccgagtacaccggaagcaccggatattccggcatgtccgagtccgggtcagaaaatcaaaaaatcaacaccggcagcaggtcagcctccgcatctgaccagcgcacaggatcaggatacaatt

SEQ ID NO: 21 - DNA, 4234 bp, Artificial sequence
  synthetic
atgaaaaggccggcggccacgaaaaaggccggccaggcaaaaaagaaaaaggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctggtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtgggaattc

SEQ ID NO: 22 - DNA, 491 bp, Artificial sequence
  synthetic
ttgatctagagggcccgcggggaggaggtggaagcggaggaggaggaagcggaggaggaggtagcagcgctgcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacggacctaagaaaaagaggaaggtggcggccgct

SEQ ID NO: 23 - DNA, 6660 bp, Artificial sequence
  synthetic
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgggataccgtctctgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttggatcctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacatgccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggacggaggaggtggaagcggaggaggaggaagcgtgagcaagggcgaggaactgttcaccggcgtggtgcccatcctggtggaactggatggcgacgtgaacggccacaagttcagcgtgtccggcgagggcgaaggcgacgccgccgctgccaagctgaccctgaagttcatctgcaccaccggcaagctgcccgtgccttggcctaccctcgtgaccacctttacctacggcgtgcagtgcttcgccagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaaggtctacatcaccgccgacaagcagaaaaacggcatcaaagtgaacttcaagacccggcacaacatcgaggacggctccgtgcagctggccgaccactaccagcagaacacccccatcggagatggccccgtgctgctgcccgacaaccactacctgagcacccagagcaagctgagcaaggaccccaacgagaagcgggaccacatggtgctgctggaatttgtgaccgccgctggcatcaccctgggcatggacgagctctacaagggatccggcgcaacaaacttctctctgctgaaacaagccggagatgtcgaagagaatcctggaccgagcgagctgattaaggagaacatgcacatgaagctctacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgaaacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgagaattcgatatcaagcttatcggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 24 - DNA, 20 bp, Artificial sequence
  synthetic
ctgccaagctgaccctgaag

SEQ ID NO: 25 - DNA, 100 bp, Artificial sequence
  synthetic
gaacggccacaagttcagcgtgtccggcgagggcgaaggcgacgccacatatggcaagctgaccctgaagttcatctgcaccaccggcaagctgcccgtg

SEQ ID NO: 26 - DNA, 20 bp, Artificial sequence
  synthetic
gtagaatgtcaacttcatga

SEQ ID NO: 27 - DNA, 20 bp, Artificial sequence
  synthetic
tactttcgcaaacctgaacg

SEQ ID NO: 28 - DNA, 20 bp, Artificial sequence
  synthetic
gtcatcttagtcattacctg

SEQ ID NO: 29 - DNA, 20 bp, Artificial sequence
  synthetic
ctctgcagaattcaaacact

SEQ ID NO: 30 - DNA, 20 bp, Artificial sequence
  synthetic
gagcccataacctctcgcgg

SEQ ID NO: 31 - DNA, 20 bp, Artificial sequence
  synthetic
aagtcctttcatttatcgga

SEQ ID NO: 32 - DNA, 100 bp, Artificial sequence
  synthetic
atgtgctagacaatatattaggtgtgggatacagtgaaatcaaatccttgagagtcagaagttgacattctactgacagaagacattcaagcaatacatg

SEQ ID NO: 33 - DNA, 18 bp, Artificial sequence
  synthetic
tgttgagtgtgcctggat

SEQ ID NO: 34 - DNA, 18 bp, Artificial sequence
  synthetic
acccttgcctctttccct

SEQ ID NO: 35 - DNA, 24 bp, Artificial sequence
  synthetic
cttgccctcatgaagttgacattc

SEQ ID NO: 36 - DNA, 27 bp, Artificial sequence
  synthetic
ccttgagagtcagaagttgacattcta

SEQ ID NO: 37 - DNA, 25 bp, Artificial sequence
  synthetic
tggaggaccgatcatgtgctagaca

SEQ ID NO: 38 - DNA, 100 bp, Artificial sequence
  synthetic
tttgctgcaaagcgtctttccctccgccccctctctgggcagcacttatcaacaggtttgcgaaagtaaagtaagtgtgccctctactggcagcagagat

SEQ ID NO: 39 - DNA, 18 bp, Artificial sequence
  synthetic
aggtggctggaaacttgt

SEQ ID NO: 40 - DNA, 19 bp, Artificial sequence
  synthetic
ggagcaaccaatcgctatg

SEQ ID NO: 41 - DNA, 19 bp, Artificial sequence
  synthetic
caaacctgaacgcgggtgc

SEQ ID NO: 42 - DNA, 24 bp, Artificial sequence
  synthetic
cgcaaacctgttgataagtgctgc

SEQ ID NO: 43 - DNA, 20 bp, Artificial sequence
  synthetic
cctcggacgctcctgctcct

SEQ ID NO: 44 - DNA, 100 bp, Artificial sequence
  synthetic
taagcaaaacatgggaactcagtttatatgagttacaacgaacacctagttcggctcctaagatgactgccaaggggcatatgagacgtgtaaactggga

SEQ ID NO: 45 - DNA, 24 bp, Artificial sequence
  synthetic
cttctttatttccagcaatgtctc

SEQ ID NO: 46 - DNA, 20 bp, Artificial sequence
  synthetic
gccaacatacagaagtcagg

SEQ ID NO: 47 - DNA, 27 bp, Artificial sequence
  synthetic
acctcaggtaatgactaagatgactgc

SEQ ID NO: 48 - DNA, 25 bp, Artificial sequence
  synthetic
ctagttcggctcctaagatgactgc

SEQ ID NO: 49 - DNA, 23 bp, Artificial sequence
  synthetic
ctgtgcagacaaacggaactcaa

SEQ ID NO: 50 - DNA, 100 bp, Artificial sequence
  synthetic
ccggcagccagaggccctttgtgaaggggagctcctcccacatataaacagtttgaattctgcaatagttctgccgttgttggattcttccatgttcatc

SEQ ID NO: 51 - DNA, 18 bp, Artificial sequence
  synthetic
gctccctcagggtctgta

SEQ ID NO: 52 - DNA, 18 bp, Artificial sequence
  synthetic
cctgtgattcagatcccg

SEQ ID NO: 53 - DNA, 26 bp, Artificial sequence
  synthetic
caagtgtttgaattctgcagagaggc

SEQ ID NO: 54 - DNA, 25 bp, Artificial sequence
  synthetic
tgaattctgcaatagttctgccgtt

SEQ ID NO: 55 - DNA, 19 bp, Artificial sequence
  synthetic
aaaggcaggtgtcctgtga

SEQ ID NO: 56 - DNA, 100 bp, Artificial sequence
  synthetic
gttttcgaagatgcgctgcacggcgaacacctgccgggtgggctcttgatgtgagaggttatgggctcccaggaccacccgcaccgcgcggacgtttctg

SEQ ID NO: 57 - DNA, 17 bp, Artificial sequence
  synthetic
ggatggggacgacaagg

SEQ ID NO: 58 - DNA, 18 bp, Artificial sequence
  synthetic
gcacctggagaatcacga

SEQ ID NO: 59 - DNA, 14 bp, Artificial sequence
  synthetic
ctcgcggcgggagc

SEQ ID NO: 60 - DNA, 24 bp, Artificial sequence
  synthetic
cataacctctcacatcaagagccc

SEQ ID NO: 61 - DNA, 22 bp, Artificial sequence
  synthetic
accccgtaaacttgctcaacga

SEQ ID NO: 62 - DNA, 103 bp, Artificial sequence
  synthetic
gcactctcaggcctcacatggcagagtatggttactgctctaaggtattaagataaatccaaggacttatttttttgagcttcagattggtggcgttattgca

SEQ ID NO: 63 - DNA, 19 bp, Artificial sequence
  synthetic
tgatgttagtgggagcagg

SEQ ID NO: 64 - DNA, 19 bp, Artificial sequence
  synthetic
cctgtctctctggaaccac

SEQ ID NO: 65 - DNA, 25 bp, Artificial sequence
  synthetic
taagtcctttcatttatcggagggc

SEQ ID NO: 66 - DNA, 30 bp, Artificial sequence
  synthetic
aataagtccttggatttatcttaatacctt

SEQ ID NO: 67 - DNA, 25 bp, Artificial sequence
  synthetic
accatgggaagtgaagaaaagcttc